(19) United States
(12) Patent Application Publication
(10) Pub. No.: US 2023/0235414 A1
(43) Pub. Date: Jul. 27, 2023

(54) ISCAN: AN RT-LAMP-COUPLED CRISPR-CAS MODULE FOR RAPID, SENSITIVE DETECTION OF SARS-COV-2

(71) Applicant: King Abdullah University of Science and Technology, Thuwal (SA)

(72) Inventors: Magdy Mahmoud Mahfouz, Thuwal (SA); Ahmed Ibrahim Mahas, Thuwal (SA)

(21) Appl. No.: 18/000,055

(22) PCT Filed: May 27, 2021
(86) PCT No.: PCT/IB2021/054664

§ 371 Date: Nov. 28, 2022

Related U.S. Application Data
(60) Provisional application No. 63/030,796, filed on May 27, 2020.

(51) Int. Cl. C12Q 1/70; C12N 9/22; C12N 15/11

(52) U.S. Cl. CPC C12Q 1/701; C12N 9/22; C12N 15/11; C12N 2310/20; C12N 2800/80

(57) ABSTRACT

Compositions and use of the compositions in methods of detecting SARS-CoV-2 in a sample is disclosed, using RT-LAMP coupled with CRISPR-Cas12, referred to herein as iSCAN (in vitro Scanning of COVID-19-Associated Nucleic acids) is disclosed. iSCAN provides a rapid, specific, accurate, sensitive detection of SARS-CoV-2 in a sample. The iSCAN is 1) rapid, as the RT-LAMP and CRISPR-Cas12/Cas 13 reaction takes less than 1 h; 2) specific, because detection depends on the identification and subsequent cleavage of SARS-CoV-2 genomic sequences by the Cas12 or 13 enzyme; 3) field-deployable, as only simple equipment is required; and 4) easy to use, as the colorimetric reaction coupled to lateral flow immunochromatography makes the assay results easy to assess. The methods include amplifying SARS-CoV-2 in a sample using RT-LAMP and using the RT-LAMP product as a substrate in a CRISPR-Cas12/13 reaction, incorporated with a means of detecting the presence of the SARS-CoV-2 RT-LAMP product.

### CROSS-REFERENCE TO RELATED APPLICATIONS

This application claims the benefit of and priority to U.S. Provisional Application No. 63/030,796 filed May 27, 2020, which is hereby incorporated by reference in its entirety.

### FIELD OF THE INVENTION

This invention is generally in the field of detecting SARS-CoV-2 in a sample.

### BACKGROUND OF THE INVENTION

In late 2019, an outbreak of the novel Severe acute respiratory syndrome-related coronavirus SARS-CoV-2, the causative agent of COVID-19, began in Wuhan, China and rapidly spread throughout the world [1-3]. Based on the data shared by WHO, as of May 4th, over three million people have tested positive for COVID-19, with a mortality rate of over 225,000 people worldwide (Ref). Coronaviruses are large, positive-stranded RNA viruses with genome sizes ranging from ˜27 to ˜32 kb [4]. These viruses pose a serious challenge to human health due to their ability to infect a wide variety of organisms, including avian and mammalian species. The rapid evolution of their genomic RNA by recombination can lead to the generation of viral strains that are more virulent or recalcitrant to therapeutic interventions than the original strains [5]. This is exemplified by the recent emergence and outbreak of the novel SARS-CoV-2.

To curb the spread of this virus, countries throughout the globe have implemented strict measures such as curfews, obligatory quarantines, social distancing, and travel bans. Although these measures have been useful in preventing significant increases in the number of new cases, they have put considerable pressure on the world economy and healthcare systems. Early detection and large-scale screening are crucial for combating emerging infectious diseases, particularly those with features characteristic of SARS-CoV-2. The facile transmissibility and delay in clinical manifestation of this virus often make it impossible for governments and healthcare providers to assess the gravity of the situation and implement preventive measures to stop the spread of the virus in a timely manner [10]. Unfortunately, as seen in too many cases, a delay in taking decisive action leads to rapid propagation of the virus within a population, where it circulates before suddenly taking on epidemic proportions [11]. From an epidemiological perspective, it is vital to rapidly identify and isolate afflicted individuals. Timely implementation of counter-measures significantly reduces the burden of the pandemic on society from both a healthcare and economic standpoint. Furthermore, the lack of testing on an appropriately large scale makes it challenging to keep track of the outbreak and to evaluate the success of the response measures. Therefore, early virus detection and isolation are crucial for identifying and isolating infected people to limit virus transmission and implementing effective measures to control the spread of the virus and relieve the burden on the healthcare system [12, 13].

Nucleic acid-based diagnostic methods are quite useful and informative compared to serological methods, which are feasible only after antibodies have been produced and only provide information about prior infection and not the current presence of the virus in a patient [14, 15]. The testing and isolation of asymptomatic carriers have proven quite effective in controlling viral spread. Therefore, efficient, low-cost testing methods are required to facilitate the extensive and recurrent testing of patients. Reverse-transcription PCR (RT-PCR) is the most widely used method for detecting RNA viruses. Due to its sensitivity and specificity, quantitative RT-PCR (RT-qPCR) is currently the gold standard method for identifying the presence of SARS-CoV-2 [1, 16]. Although this method is sensitive and reliable, is not without limitations. RT-qPCR requires highly trained personnel, sophisticated infrastructure, and the transport of samples to central laboratories, thereby limiting its usefulness for large-scale point-of-care diagnostics [17]. In addition, this technique is difficult to implement during emergency situations such as the SARS-CoV-2 pandemic, when thousands of samples must be analyzed as quickly as possible to assess treatment options and prevent further spread of the disease [18]. Therefore, methods that conform to the ASSURED features of the point-of-care (POC) diagnostic are urgently needed to mitigate the spread of SARS-CoV-2 through effective control and management measures [19].

Several approaches have been employed for pathogen diagnostics using inexpensive reagents and simple equipment, leading to effective testing in low-resource settings. For example, isothermal amplification approaches based on recombinase polymerase amplification or loop-mediated isothermal amplification (LAMP), which are conducted at a single temperature, have been employed for POC diagnostics for various pathogens, thus providing a very effective alternative to PCR-based methods [14, 20]. Reverse-transcription LAMP (RT-LAMP), which uses a suitable isothermal reverse transcriptase to reverse transcribe a specific sequence of virus RNA to DNA coupled with isothermal reactions such as LAMP, can be used to detect RNA viruses in a single reaction at a single temperature [21]. However, these methods have some drawbacks, including low specificity, a high rate of false positives, and difficulty of adapting these techniques for effective POC diagnostics for pandemics such as COVID-19 [22, 23].

CRISPR-Cas systems are molecular immunity machines that provide bacterial and archaeal species with immunity against invading nucleic acids, including phages and conjugative plasmids [24, 25]. These systems have been used to engineer the genomes and transcriptomes of all transformable species [26-28]. One intriguing feature of some of these systems, including those employing variants of Cas12, Cas13, and Cas14, is their collateral, non-specific activity following recognition and cleavage of the specific target [29-31]. CRISPR-Cas systems, including CRISPR-Cas12 and CRISPR-Cas13, exhibit robust collateral activity against single-stranded DNA (ssDNA) and RNA targets, respectively. Such collateral activity provides the basis for highly specific, sensitive approaches for nucleic acid detection [32, 33]. For example, SHERLOCK (Specific High-sensitivity Enzymatic Reporter UnLOCKing) and DETECTR (DNA Endonuclease-Targeted CRISPR Trans Reporter) detection have attracted substantial attention [30, 34]. CRISPR-based diagnostic methods exploit the efficiency and simplicity of different isothermal amplification approaches, such as LAMP and recombinase polymerase amplification, which achieve highly specific, sensitive amplification of a few copies of the targeted nucleic acid at single temperature in a short period of time, eliminating the need for thermocycling steps, and are therefore favored for POC diagnostics where low-cost and ease of use are needed. Although these systems hold great promise for developing an effective diagnostic, employing these techniques as POC diagnostics amenable to massive-scale field deployment remains challenging.

It is an object of the present invention to provide compositions, methods, and kits for detecting and diagnosing SARS-CoV-2.

### SUMMARY OF THE INVENTION

Compositions and use of the compositions in methods of detecting bacterial or viral (e.g., SARS-CoV-2) nucleic acids in a sample are disclosed. An approach has been developed using RT-LAMP coupled with a CRISPR/Cas effector protein (e.g., CRISPR-Cas12), referred to herein as iSCAN (in vitro Scanning of COVID-19-Associated Nucleic acids) to provide rapid, specific, accurate, and sensitive detection of SARS-CoV-2 in a sample. The iSCAN method is 1) rapid, as the RT-LAMP and CRISPR-Cas12 or Cas13 reactions together can take less than 1 h; 2) specific, because detection depends on the identification and subsequent cleavage of SARS-CoV-2 genomic sequences by the Cas12 or 13 enzyme; 3) field-deployable, as only simple equipment is required; and 4) easy to use, as for example, a colorimetric reaction coupled to lateral flow immunochromatography makes the assay results easy to assess.

The method typically includes amplifying SARS-CoV-2 in a sample using RT-LAMP and using the RT-LAMP amplification product (or a product derived therefrom) as a substrate in a CRISPR-Cas12 or -Cas13 reaction, incorporated with a means of detecting the presence of the SARS-CoV-2 RT-LAMP product. In some embodiments, the sample is processed to expose or isolate nucleic acids (e.g., RNA) from the sample before it is subjected to the detection method.

The compositions can include RT-LAMP reagents, an RNA polymerase, a CRISPR Cas12 or Cas13 reaction system, and an activatable oligonucleotide or probe, which includes a reporter moiety. Exemplary RT-LAMP reagents include RT-LAMP primers, for example, SEQ ID Nos. 11-28 and SEQ ID Nos. 29-44 for amplifying the N or E genes of SARS-CoV-2, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing; aDNA polymerase with strand displacement activity and RT-LAMP buffers. In a preferred embodiment, the primers include SEQ ID Nos. 17-22, or a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing.

The CRISPR-Cas reaction system includes purified Cas12 or Cas 13 assembled with crRNAs targeting dsDNA fragments (or RNA transcripts derived therefrom) of the SARS-CoV-2 N (nucleoprotein) or E (envelope) genes, preferably, the N gene, and more preferably, the N2 region in the N gene. In a preferred embodiment, the Cas system uses Cas12, preferably Cas 12a or 12b (e.g., LbCas12a, AacCas12b or AapCas12b). In another preferred embodiment, the Cas system uses Cas13, preferably TccCas13a (SEQ ID NO:67), HheCas13a (SEQ ID NO:95), or mCas13 (SEQ ID NO:68). Preferably, the crRNA is designed to hybridize to the RT-LAMP product. The activatable oligonucleotide is preferably a labelled ssDNA or ssRNA reporter, preferably, a non-targeted fluorophore quencher (FQ)-labeled ssDNA or ssRNA reporter.

The disclosed compositions can be used in methods of detecting SARS-CoV-2 nucleic acids in a sample such as mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood.

In a first step in some embodiments of the disclosed methods, RT-LAMP is performed to reverse transcribe and pre-amplify the viral RNA to generate dsDNA substrates for Cas12 enzymes, for example, Cas12a or Cas12b. The RT-LAMP product is used as a target for the CRISPR-Cas12 system. Purified Cas12 proteins are assembled with crRNAs targeting dsDNA fragments of the SARS-CoV-2 N or E genes. The Cas 12/crRNA reaction with the RT-LAMP product is carried out in the presence of an activatable probe/molecular beacon, which is preferably, single stranded DNA (ssDNA) oligonucleotide (herein collateral ssDNA reporter). Collateral ssDNA reporter is cleaved upon Cas12a binding and cleavage of the target virus sequence in the RT-LAMP amplification products, resulting in release of the quenched signal in the molecular beacon/activatable probe.

In a specific embodiment, a method of detecting the presence of SARS-CoV-2 nucleic acid in a sample involves (a) contacting the sample with a set of RT-LAMP primers specific for the N or E gene of SARS-CoV-2 in an RT-LAMP reaction under conditions sufficient for amplification of the N or E gene of SARS-CoV-2 to generate an amplification product, (b) contacting the amplification product with (i) a composition of a ribonucleoprotein (RNP) complex including a DNA editing enzyme having both dsDNA and ssDNA cleavage activity and a crRNA complementary to the amplification product and (ii) an activatable ssDNA oligonucleotide containing a reporter moiety, and (c) detecting cleavage of the ssDNA oligonucleotide by the DNA editing enzyme, wherein the cleavage is dependent on or subsequent to binding of the RNP to the RT-LAMP amplification product, thereby detecting the presence of the SARS-CoV-2 nucleic acid.

In some embodiments of the disclosed methods, the amplification product from the RT-LAMP reaction is transcribed to generate ssRNA substrates for a Cas13 enzyme, for example, TccCas13a or mCas13. The ssRNA substrate is used as a target for the CRISPR-Cas13 system. For example, in a specific embodiment, a method of detecting the presence of SARS-CoV-2 nucleic acid in a sample involves (a) contacting the sample with a set of RT-LAMP primers specific for the N or E gene of SARS-CoV-2 in an RT-LAMP reaction under conditions sufficient for amplification of the N or E gene of SARS-CoV-2 to generate an amplification product; (b) transcribing the amplification product to generate an RNA transcript; (c) contacting the RNA transcript with (i) a composition of a Cas13-based RNP complex composed of a Cas13 enzyme and a crRNA complementary to the RNA transcript; and (ii) an activatable single stranded RNA (ssRNA) oligonucleotide; and (d) detecting cleavage of the ssRNA oligonucleotide by the Cas13 enzyme. In some embodiments, the cleavage of the ssRNA oligonucleotide is dependent on or subsequent to binding of the Cas13-based RNP to the RNA transcript. In some embodiments, cleavage of the ssRNA oligonucleotide results in release of a quenched fluorescent signal, thereby indicating the presence of the SARS-CoV-2 nucleic acid.

Although discussed herein most typically in terms of detecting SARS-CoV-2, it will be appreciated, that the methods can be adapted for detection of any target nucleic acid, and thus such compositions and methods are also disclosed, and all materials and methods disclosed for use with SARS-CoV-2 can also disclosed for detection of other target nucleic acids, or can be modified for detection thereof. In a specific embodiment, disclosed is a method of detecting the presence of any target nucleic acid in a sample. Typically, the method involves, (a) contacting the sample in an RT-LAMP reaction with a set of primers specific to the target nucleic acid under conditions sufficient for amplification of the target nucleic acid to generate an amplification product; (b) transcribing the amplification product to generate an RNA transcript; (c) contacting the RNA transcript with (i) a RNP complex comprising a Cas13 enzyme comprising the sequence of any one of SEQ ID NO:67, 68, or 95 or a sequence having at least 70% sequence identity to any one of SEQ ID NO:67, 68, or 95, and a crRNA complementary to the RNA transcript; and (ii) an activatable single stranded RNA (ssRNA) oligonucleotide; and (d) detecting cleavage of the ssRNA oligonucleotide by the Cas13 enzyme, thereby detecting the presence of the target nucleic acid.

In some embodiments, the target nucleic acid can be associated with a pathogen (e.g., a pathogenic bacterium, such as Salmonella, Listeria and E. coli). In some embodiments, the target nucleic acid can be associated with a disease, such as an infection, an organ disease, a blood disease, an immune system disease, a cancer, a brain and nervous system disease, an endocrine disease, a pregnancy or childbirth-related disease, an inherited disease, or an environmentally-acquired disease. In some embodiments, the target nucleic acid can be associated with a fungal infection, a bacterial infection, a parasitic infection, a viral infection, or combination thereof.

The disclosed RT-LAMP CRISPR/Cas12 or Cas13 coupled detection assays are highly sensitive. Preferably, the methods are capable of detecting a target nucleic acid, such as SARS-CoV-2 nucleic acid, when present in a concentration about 2-20 copies/μL (e.g., about 4-8 copies/μL or 5-10 copies/μL).

The disclosed approaches are suitable for large-scale, in-field deployment for the early detection of SARS-CoV-2 carriers, allowing them to be effectively isolated and quarantined, thus limiting the spread of the virus. The SARS-CoV-2 detection assays have been validated using clinical samples, providing the possibility of widespread deployment for virus detection.

### BRIEF DESCRIPTION OF THE DRAWINGS

FIGS. 1A-1D show visualization of enzyme activity and performance of the in-house produced proteins. FIG. 1A. Visualization of the amplicons in agarose gel after RT-LAMP amplification of E-gene; E-T: E gene detection in Tris buffer, E-H: E gene detection in HEPES buffer, +ve: E gene detection with commercial enzymes, N1: sample without crRNA, N2: sample with NS-crRNA, NTC: no-template control. FIG. 1B. Cas12a based restriction of target dsDNA. Visualization of the fragments generated by Cas12a cis-cleavage of the target regions in agarose gel. FIG. 1C. Visual fluorescence readout after Cas12a collateral activity; E-T: E gene detection in Tris buffer, E-H: E gene detection in HEPES buffer, +ve: E gene detection with commercial enzymes, N1: sample without crRNA, N2: sample with NS-crRNA, NTC: no-template control. FIG. 1D. Quantification of fluorescence signal after Cas12a collateral cleavage of reporters; E-T: E gene detection in Tris buffer, E-H: E gene detection in HEPES buffer, +ve: E gene detection with commercial enzymes, N1: sample without crRNA, N2: sample with NS-crRNA, NTC: no-template control.

FIG. 2A-2G show CRISPR-Cas12a-based iSCAN assay for detection of SARS-CoV-2. FIG. 2A shows the SARS-CoV-2 genome architecture. Regions targeted by iSCAN assay are highlighted. FIG. 2B shows workflow of iSCAN detection assay. FIG. 2C shows quantifications of signal intensities of CRISPR-Cas12a fluorescence-based detection assays of synthetic SARS-CoV-2. E-T: E gene with Tris buffer, E-H: E gene with HEPES buffer, +ve: E gene with commercial buffer (NEB), N1: no crRNA, N2: NS-crRNA, NTC: no-template control. FIG. 2D shows limit of detection (LoD) determination of RT-LAMP-Cas12a assay. Values shown as mean±SEM (n=4). FIG. 2E shows end-point fluorescence visualization under UV light following the CRISPR/Cas12a detection assay performed on clinical samples. S: different clinical samples with different Ct values, +Ve: Synthetic RNA, NTC: no-template control. FIG. 2F shows a lateral flow readouts of Cas12a detection of SARS-CoV-2 RNA in clinical samples. FIG. 2G shows end-point fluorescence intensity measured in clinical samples after CRISPR/Cas12a detection assay. FIG. 2H shows RT-LAMP and Cas12a time course and optimization of assay duration. The figure depicts different combinations of RT-LAMP assay and Cas12a incubation times. NTC: no-template control.

FIGS. 3A-3C show detection of SARS-CoV-2 RNA extracted from patient samples. FIG. 3A show RT-LAMP generated amplification products with primer set targeting the E gene in agarose gel; S-2, S-5, S-5, S-6, S-8, S-15, S-12, S-32: RNA isolated from clinical samples; +ive: positive control with commercial enzymes; NTC: no-template control. FIG. 3B shows fluorescence signal of the samples after Cas12a-based detection assay; E-crRNA: assay performed with E gene crRNA, NS-crRNA: assay performed with non-specific crRNA. FIG. 3C shows fluorescence signal of the samples after Cas12a-based detection assay; E-crRNA: assay performed with E gene crRNA, NS-crRNA: assay performed with non-specific crRNA. FIGS. 3D-3E show detection of SARS-CoV-2 RNA by targeting the N gene. FIG. 3D is a visual readout of the results using lateral flow strips. FIG. 3E shows visualization of RT-LAMP generated amplification products in an agarose gel. S01-S24 are patient samples, +ive is the positive control and −ive is the no-template control. FIG. 3F-3G show AapCas12b and AacCas12b cis and trans cleavage activity. FIG. 3F. AapCas12b and AacCas12b cis cleavage activity assay performed at 62° C. for 30 minutes. FIG. 3G. AapCas12b and AacCas12b trans cleavage activity assay. Values shown as mean SD (n=3).

FIGS. 4A-4D show CRISPR-Cas12b-based assay for detection of SARS-CoV-2. FIG. 4A shows limit of detection (LoD) of RT-LAMP-AapCas12b assay in spotted one-pot reaction. Values shown as mean±SEM (n=3). FIG. 4B shows visual fluorescence output of four clinical samples under UV light following CRISPR/Cas12b detection assay performed by two different strategies. S: different clinical samples with different Ct values. FIG. 4C shows end-point fluorescence intensity measured in clinical samples after performing spotted one-pot CRISPR-AapCas12a detection assay. S: different clinical samples with different Ct values, +Ve: Synthetic RNA, NTC: no-template control. FIG. 4D shows lateral flow readout after spotted one-pot AapCas12b detection assay performed on clinical samples.

FIGS. 5A and 5B show an overview of the CRISPR-Cas based SARS-CoV-2 detection methods and requirements. FIG. 5A is a schematic illustration showing different Cas12-based detection modalities. The first row depicts Cas12a-based two-pot reaction, where the RT-LAMP amplification of SARS-CoV-2 nucleic acid is performed in the first tube. Then the RT-LAMP product is transferred to a second tube for Cas12a-based detection. The second row depicts RT-LAMP and Cas12b-based detection reagents, except for Cas12b-sgRNA complex, mixed in a single reaction. Cas12b-sgRNA complex is temporary separated from the reaction because it is added on the tube wall. After 30 min of RT-LAMP reaction, the Cas12b-sgRNA complex is centrifuged into the reaction mix for target cleavage and CRISPR/Cas-based detection, which takes place for an additional 15 min. In the third row, simultaneous RT-LAMP amplification and CRISPR-based detection of SARS-CoV-2 detection is performed in a single tube. FIG. 5B shows the minimal equipment and consumables required for iSCAN detection assay, which includes a heat block, pipettes, pipette tips, sample tubes, and lateral flow strips.

FIGS. 6A and 6B are heatmaps showing the screening and selection of the optimal ssRNA reporter for TccCas13a and HheCas13a in-trans activity. Reactions containing Cas13 and two different crRNAs targeting the SARS-CoV-2 N gene or non-specific crRNA (NS) control were performed in the presence of one of three FAM-reporters with different RNA sequences. Data are shown as the mean of three replicates.

FIGS. 7A and 7B are graphs showing the in-trans activity of HheCas13a and TccCas13a assessed at varying target concentrations and two different temperatures. FIG. 7C is a heatmap depicting the in-trans activity of HheCas13a and TccCas13a assessed at different temperatures with two crRNAs targeting the N SARS-CoV-2 gene and one non-specific (NS) crRNA. Reactions were run for 30 mins. Data are shown as mean with n=3. FIG. 7D is a bar graph showing the in-trans activity of TccCas13a assessed at 60° C. with two crRNAs of different spacer lengths targeting the N SARS-CoV-2 gene and one non-specific (NS) crRNA. Reactions were run for 30 mins. Values shown as mean±SD (n=3).

FIGS. 8A-8D are bar graphs showing the results of the screening of the indicated HheCas13a and TccCas13a crRNAs paired with various primers. crRNAs designed to target the RNA substrate produced from T7-mediated in vitro transcription of the RT-LAMP amplification products using STOPCovid (SC) primer sets with T7 promoter appended to the FIP primer (T7-FIP) or to the BIP primer (T7-BIP). RT-LAMP amplification was performed first and the RT-LAMP product was added to the T7-in vitro transcription and Cas13-based detection reaction (two-pots). The in-trans activity of HheCas13a and TccCas13a were assessed at 56° C. NS: non-specific crRNA. Reactions were run for 30-60 mins.

FIGS. 9A-9D are bar graphs showing the results of the screening of the indicated HheCas13a and TccCas13a crRNAs paired with various primers in the one-pot assay. crRNAs designed to target the RNA substrate produced from T7-mediated in vitro transcription of the RT-LAMP amplification using STOPCovid (SC) primer sets with T7 promoter appended to the FIP primer (T7-FIP) or to the BIP primer (T7-BIP). RT-LAMP amplification, T7-in vitro transcription and Cas13-based detection was performed in single step at single temperature, 55° C. (one-pot). NS: non-specific crRNA. Reactions were run for 30-60 mins. Values shown as mean±SD (n=3).

FIGS. 10A-10E are graphs showing the results from a second round of screening of the indicated HheCas13a and TccCas13a crRNAs and different primer sets for the establishment of one-pot assay. RT-LAMP amplification, T7-in vitro transcription and Cas13-based detection was performed in single step at single temperature, 55° C. (one-pot). NS: non-specific crRNA. Reactions were run for 80 mins. Values shown as mean (n=3).

FIG. 11 is a heatmap showing the in-trans activity of the one-pot detection assay with synthetic RNA using different Bst DNA polymerases from different vendors. NTC: non-template control. Reactions were run for 80 mins at 55° C. Values shown as mean (n=3).

FIGS. 12A-12D are bar graphs showing the cleavage activity towards the reporter probe in the one-pot assay with different concentrations of Bst DNA polymerase (FIG. 12A), MgSO4 (FIG. 12B), Hi-T7 RNA polymerase (FIG. 12C), and TccCas13a and crRNA (RNP) concentrations (FIG. 12D). NTC: non-template control. Reactions were run for 80 mins at 55° C. Values shown as mean±SD (n=3).

FIG. 13 is a bar graph quantifying the limit of detection (LoD) of the thermophilic Cas13-based one pot detection assay using synthetic SARS-CoV-2 RNA as an input. Reactions were run for 80 mins at 56° C. Values shown as mean±SD (n=3).

FIG. 14 is a graph quantifying the performance of the thermophilic Cas13-based one-pot detection assay on SARS-CoV-2 patient samples. RNA extracted from clinical samples from 8 patients with SARS-CoV-2 infection (Table 10) were analyzed with the one-pot detection assay. NTC: no template control. Reactions were run for 80 mins at 56° C.

FIG. 15A is a schematic of specific Cas13-based detection. Specific target recognition by Cas13 RNP triggers non-specific, collateral activity that cleaves the reporters, resulting in a detectable fluorescent signal. FIGS. 15B-15F are bar graphs showing cleavage activity across various reporter and crRNA sequences. Reactions containing mCas13 and four different crRNAs targeting the SARS-CoV-2 N gene or non-specific crRNA (NS) control were performed in the presence of one of five reporters. F-NNNNN-Q represents the RNaseAlert v2 reporter (Thermofisher). NS: non-specific (not targeting SARS-CoV-2) crRNA. Values shown as mean±SD (n=3). FIG. 15G is a bar graph quantifying the effect of mismatches between mCas13 crRNA and target RNA on mCas13 activity. Left, crRNA nucleotide sequence with the positions of mismatches (red) on the crRNA. Right, the fluorescence intensity, relative to the no-template control (NTC, gray) or crRNA with no mismatches (purple), resulting from mCas13 collateral cleavage activity on each tested crRNA. FIG. 15H is a schematic illustration of the sequence similarity searching strategies for the identification of miniature Cas13 effectors. BLASTp analysis was performed before January 2021. FIG. 15I is a bar graph showing titration of mCas13 and crRNA concentrations for optimal performance 100 nM of in vitro transcribed RNA of SARS-CoV-2 N gene was used as the target sequence in mCas13 reactions. Values represent endpoint fluorescence at 30 mins. Values shown as mean±SD (n=3). FIG. 15J is a bar graph quantifying mCas13 collateral activity at the indicated temperatures. Collateral activity was measured as end point fluorescence after incubation with 100 nM of in vitro transcribed RNA of SARS-CoV-2 N gene for 45 mins. Values shown as mean±SD (n=3). FIG. 15K shows multiple sequence alignment of mCas13 and compact Cas13e and Cas13f orthologues. Protein sequences of mCas13, Cas13e.1, Cas13e.2, Cas13f.1, Cas13f.2, Cas13f.3, Cas13f.4 and Cas13f.5 were aligned using ClustalW in MEGAX and ESPript was used to generate the alignment visualization. Strictly conserved residues (% Strict) are shown in red text within blue rectangles. Conserved RxxxxH motifs of the HEPN domains are highlighted with yellow background.

FIG. 16A is a schematic of assay workflow of mCas13-based detection. Following sample collection and RNA extraction, pathogen RNA is reverse transcribed and amplified via RT-LAMP isothermal reaction. RT-LAMP primer sets (in which the FIP contains a T7 promoter sequence) are used, resulting in amplicons containing the T7-promoter sequence, which serve as templates for in vitro transcription of target RNA. Upon target recognition, mCas13 cleaves specially designed reporters in trans, leading to fluorescent signal output. FIG. 16B is a graph showing measurement of real time fluorescence output of T7-mediated in vitro transcription and mCas13-based detection. Synthetic SARS-CoV-2 RNA was reverse transcribed and LAMP amplified with STOPCovid (SC) and DETECTR (DT) primer sets. crRNA 4: targeting crRNA #4 identified in FIG. 15. NS: non-specific crRNA. Values shown as mean (n=3) FIGS. 16C-16D are bar graphs showing the limit of detection (LoD) of mCas13-based detection assay. LoD was determined using synthetic SARS-CoV-2 RNA, which was reverse transcribed and LAMP-amplified with STOPCovid and DETECTR primer sets. LAMP product was subsequently used for T7 in vitro transcription and concurrent mCas13 detection. Values shown as mean±SD (n=3). FIG. 16E is a graph showing results of the mCas13-based detection assay across different viruses. Detection of non-specific viral targets including SARS-CoV-1, MERS-CoV, as well as two plant viruses, tobacco mosaic virus (TMV) and turnip mosaic virus (TuMV) was attempted using the STOPCovid primer set. Values shown as mean±SD (n=3).

FIG. 17A is a graph showing validation of mCas13-based detection assay on RT-qPCR-validated SARS-CoV-2 clinical samples with different Ct values. Pink bars represent mCas13-based detection fluorescence output. FAM: RNA reporter labeled with FAM fluorophore used in mCas13 detection assays. Blue dots represent N gene Ct values. FIG. 17B is a schematic representation of mCas13-based visual detection with a handheld fluorescence visualizer. FIGS. 17C-17D are images showing visual readouts of the limit of detection (LoD) of mCas13-based detection assay. LoD was determined using synthetic SARS-CoV-2 RNA (FIG. 17C) or RT-qPCR-validated SARS-CoV-2 clinical samples with different Ct values (FIG. 17D), which were reverse transcribed and LAMP-amplified with the STOPCovid primer set. LAMP product was used for T7 in vitro transcription and concurrent mCas13 detection. FIG. 17E is a series of images showing fluorescence readouts of RT-LAMP mCas13 visual detection reactions of 24 SARS-CoV-2 RT-qPCR positive samples and no template control (NTC) reactions used to generate the heatmap in FIG. 17F. Left, reaction tubes are shown with the clinical sample ID. Right, reaction tubes are shown with RT-qPCR Ct values indicated. FIG. 17F is a heat map displaying the validation of the mCas13-based visual detection assay of 24 SARS-CoV-2 RT-qPCR positive samples. RFU: random fluorescence units, showing the signal intensity that was obtained by the TECAN plate reader for the mCas13 reactions.

### DETAILED DESCRIPTION OF THE INVENTION

### I. Definitions

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclose

The term “amplification” as used herein refers to increasing the number of copies of a nucleic acid molecule, such as a gene or fragment of a gene, for example at least a portion of the SARS-CoV-2 RNA or DNA copy thereof. The products of an amplification reaction are called amplification products or amplicons. An example of in vitro amplification is loop-mediated isothermal amplification.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “conditions sufficient for” as used herein in connection with the disclosed methods, refers to any environment that permits the desired activity, for example, that permits specific binding or hybridization between two nucleic acid molecules or that permits reverse transcription and/or amplification of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), etc.).

The term “contact” as used herein in connection with the disclosed methods refers to placement in direct physical association; for example, in solid and/or liquid form. For example, contacting can occur in vitro with one or more primers and/or probes and a biological sample (such as a sample including nucleic acids) in solution.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “sample” refers to in vitro as well as clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material), nasopharyngeal aspirate, nasopharyngeal swab, throat swab, and other discussed herein and otherwise known in the art.

“Sensitivity” as used herein, is a measure of ability of a detection assay to directly or indirectly detect the presence of a target sequence (e.g., a SARS-CoV-2 viral sequence) in a sample.

“Specificity,” as used herein, is a measure of the ability of a detection assay to distinguish a truly occurring target sequence (e.g., a SARS-CoV-2 viral sequence) from other closely related sequences (e.g., other human-pathogenic coronaviruses and respiratory pathogens). It is the ability to avoid false positive detections.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

“Unit” or “U” when used in context of an enzyme refers to an amount of enzyme required to convert a given amount of reactant or substrate to a product using a defined time and temperature.

As used herein, the term “thermostable enzyme” refers to an enzyme which is stable to heat and/or is heat resistant. The enzyme is not irreversibly denatured (inactivated) when subjected to elevated temperatures. Irreversible denaturation refers to permanent and complete loss of enzymatic activity. The heating conditions necessary for denaturation will depend, e.g., on the buffer, salt concentration, nucleotide composition of the nucleic acids being acted upon, etc.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988). Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). In some forms, the default parameters can be used to determine the identity for the polynucleotides or polypeptides of the present disclosure.

In some forms, the % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

### II. Compositions

Compositions useful for detecting specific nucleic acids (e.g., bacterial or viral nucleic acid, such as SARS-CoV-2 RNA) in a sample are described. Typically, the compositions include reagents suitable for performing reverse transcription, isothermal amplification (e.g., LAMP), transcription, and/or enzymatic (e.g., CRISPR/Cas mediated) nucleic acid cleavage and detection thereof.

In some embodiments, the compositions include reagents for performing coupled reverse transcription and isothermal amplification of a nucleic acid, such as RT-LAMP. In preferred embodiments, the compositions include reagents for performing coupled reverse transcription and isothermal amplification of SARS-CoV-2 RNA by RT-LAMP.

### A. Reverse Transcriptase

LAMP can be used for amplification of RNA targets with the addition of reverse transcriptase (RT) to the reaction without an additional heat step (referred to as RT-LAMP). The term “reverse transcriptase” describes a class of enzymes characterized as RNA-dependent DNA polymerases. All known reverse transcriptases require a primer to synthesize a DNA transcript from an RNA template. Three different approaches can be used for priming RT reactions: oligo(dT) primers, random primers, or sequence specific primers. In some embodiments, two or all three approaches are combined. Oligo(dT) or primers anneal to the polyA tail of, for example, template mRNA or genomic RNA strand.

Historically, reverse transcriptase has been used primarily to transcribe total RNA or mRNA in particular into cDNA. Thus, RT can be used to first generate a DNA complement of an initial RNA sample before the amplification procedure. The template RNA can be SARS-CoV-2 RNA, e.g., genomic RNA, mRNA, or a combination thereof.

The RT may be obtained from any source and can be a native or recombinant protein. In some embodiments, the RT can be commercially obtained.

Exemplary reverse transcriptases that can be used include, without limitation, MMLV reverse transcriptase, AMV reverse transcriptase, ThermoScript™ reverse transcriptase (Life Technologies, Grand Island, N.Y.), Thermo-X™ reverse transcriptase (Life Technologies, Grand Island, N.Y.), RTx reverse transcriptase, and the SuperScript™ line of reverse transcriptases (SuperScript™ I-IV).

In some embodiments, about 0.1 to 50 U (such as about 0.2 to 40 U, about 0.5 to 20 U, about 1 to 10 U, about 2 to 8 U, or about 5 U) of RT is included in the reaction.

### B. Amplification Primers

In preferred embodiments, the compositions include RT-LAMP primers for amplifying one or more regions of the SARS-CoV-2 genome, preferably the N and/or E genes of SARS-CoV-2. The primers can be designed to recognize distinct target sequences, for example distinct parts of the SARS-CoV-2 N or E genes. In some embodiments, the primers are designed to amplify one or more regions of the SARS-CoV-2 genome that are highly conserved among coronaviruses. In some embodiments, the primers are designed to amplify one or more regions of the SARS-CoV-2 genome that allow it to be distinguished from other viruses, such as SARS-CoV-1, MERS-CoV, tobacco mosaic virus (TMV), or turnip mosaic virus (TuMV).

The SARS-CoV-2 genome consists of ˜30 kb positive single-stranded RNA with a 5′-cap structure and 3′ poly-A tail containing several genes characteristic of coronaviruses, such as S (spike), E (envelope), M (membrane), and N (nucleocapsid) genes. Other elements of the genome, such as ORF1a and ORF1b, encode non-structural proteins, including RNA-dependent RNA polymerase (RdRp) [8, 9]. In preferred embodiments, the primers amplify N1 or N2 regions of the N gene. The genome location of N2 region is known in the art. See for example, nucleotide numbering based on SARS-CoV-2 (GenBank accession no. MN908947), Lu, et at, Emerg Infect Dis. 2020 August https://doi.org/10.3201/eid2608.201246.

In some embodiments, the LAMP primers are used to generate ˜200 bp amplification products to ensure robust amplification sufficient for LAMP-based detection. However, the primers can be designed to generate amplicons of various sizes, such as, but not limited to, about 100 bp, about 150 bp, about 200 bp, about 250 bp, about 300 bp, or more.

Primers are typically at least 10, 15, 18, 20, 30, 40, 50, or 60 nucleotides in length. In some embodiments, the primers are between 10 and 60 nucleotides in length, such as 10, 11, 12, 13, 14, 15, 16, 17, 18, 19, 20, 21, 22, 23, 24, 25, 26, 27, 28 29, 30, 31, 32, 32, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. In some embodiments, the primers are at least 10, 15, 18, 20, 25, 30, 35, 40, 45, 50, 55, or 60 nucleotides in length. In other embodiment, the primers and/or probes may be no more than 10, 15, 18, 20, 25, 30, 35, 40, 45, 50, 55, or 60 nucleotides in length. However, there is no standard primer length for optimal hybridization or amplification. An optimal length for a particular primer application may be readily determined by those of skill in the art.

Typically, at least four primers are used for LAMP. The four primers include a forward outer primer (F3), a backward outer primer (B3), a forward inner primer (FIP), and a backward inner primer (BIP). A forward loop primer (LF), and a backward loop primer (LB) can also be further included in some embodiments. Thus, at least six primers are used for LAMP in some embodiments. The amplification reaction produces a stem-loop DNA with inverted repeats of the target nucleic acid sequence.

Exemplary primer sequences for amplifying the N or E genes of SARS-CoV-2 are listed in Tables 4 and 5. Exemplary primer sequences include SEQ ID NOs:11-44, sequences having at least 70%, 75%, 80%, 85%, 90%, 95%, 96%, 97%, 98%, or 99% sequence identity thereto, nucleic acid sequences having 1, 2, 3, 4, 5, 6, 7, 8, 9, or 10 nucleic acid substitution(s), addition(s), deletion(s), or a combination thereof relative thereto, or the reverse complement of any of the foregoing. In a preferred embodiment, suitable primers include SEQ ID NOs:17-28 (e.g., 17-22), or sequences having at least 70%, 75%, 80%, 85%, 90%, 95%, 96%, 97%, 98%, or 99% sequence identity thereto, nucleic acid sequences having 1, 2, 3, 4, 5, 6, 7, 8, 9, or 10 nucleic acid substitution(s), addition(s), deletion(s), or a combination thereof relative thereto, or the reverse complement of any of the foregoing.

In some embodiments, the primers preferably include SEQ ID NOs:29-44 or sequences having at least 70%, 75%, 80%, 85%, 90%, 95%, 96%, 97%, 98%, or 99% sequence identity thereto, nucleic acid sequences having 1, 2, 3, 4, 5, 6, 7, 8, 9, or 10 nucleic acid substitution(s), addition(s), deletion(s), or a combination thereof relative thereto, or the reverse complement of any of the foregoing. For example, in some embodiments, a set of primers collectively include SEQ ID NOs:29-36. In some embodiments, a set of primers collectively include SEQ ID NOs:37-44.

In some embodiments, for example when the amplification product is to be transcribed into an RNA transcript for CRISPR/Cas recognition, the FIP or BIP primer can be designed to include the T7 promoter sequence appended at the 5′ end of the first half of the primer. Examples of such modified primer sequences include SEQ ID NOs:35, 36, 43 and 44.

### C. DNA Polymerase

The compositions also include a DNA polymerase to amplify the target nucleic acid. Preferably, the DNA polymerase has strand displacement activity and can release the newly formed DNA strands. Preferably, the DNA polymerase is a thermostable enzyme (e.g., not being irreversibly denatured or inactivated when subjected to the elevated temperatures for certain steps in an amplification procedure). The DNA polymerase may be obtained from any source and can be a native or recombinant protein. In some embodiments, the DNA polymerase can be commercially obtained.

Exemplary preferred DNA polymerases include Bst DNA polymerase, Bst 2.0 DNA polymerase, Bst 2.0 WarmStart™ DNA polymerase (New England Biolabs, Ipswich, Mass.), Bst 3.0 DNA polymerase (New England Biolabs, Ipswich, Mass.). Other exemplary DNA polymerases include Phi29 DNA polymerase, Bsu DNA polymerase, OmniAmp™ DNA polymerase (Lucigen, Middleton, Mich.), and Taq DNA polymerase.

In some embodiments, about 1 to 20 U (such as about 1 to 15 U, about 2 to 12 U, about 10 to 20 U, about 2 to 10 U, about 5 to 10 U, or about 8 U) of DNA polymerase is included in the RT-LAMP reaction. In some embodiments, the polymerase lacks 5′-3′ exonuclease activity. In a preferred embodiment, the DNA polymerase is Bst DNA polymerase.

### D. RNA Polymerase

In some embodiments, an RNA polymerase can also be provided in the compositions. The RNA polymerase can be used to generate an RNA transcript from the DNA amplification product generated by RT-LAMP. The RNA transcript can be subsequently recognized, bound, and/or cleaved by an appropriate CRISPR-Cas based ribonucleoprotein (RNP) complex.

In some embodiments, the RNA polymerase is a thermostable enzyme. The RNA polymerase may be obtained from any source and can be a native or recombinant protein. In some embodiments, the RNA polymerase can be commercially obtained.

Non-limiting examples of suitable RNA polymerases include phage-derived RNA polymerases, such as, T7 RNA polymerase (e.g., Hi-T7 RNA polymerase), T3 RNA polymerase, and SP6 RNA polymerase. In a preferred embodiment, Hi-T7® RNA Polymerase (New England Biolabs, Catalog #: M0658S) is used.

### E. CRISPR/Cas RNP complex

The compositions can also include a CRISPR/Cas effector protein or enzyme and an associated crRNA (together referred to as an “RNP complex”). CRISPR/Cas systems, including CRISPR-Cas12 and CRISPR-Cas13, exhibit robust collateral activity against single-stranded DNA (ssDNA) and ssRNA targets, respectively. The collateral, cleavage of a non-specific target following recognition and cleavage of the specific target by the Cas effector protein (complexed with crRNA) provides the basis for highly specific, sensitive approaches for nucleic acid detection.

In some embodiments, the CRISPR/Cas effector is a DNA editing enzyme (e.g., DNA endonuclease) with dsDNA cleavage activity and ssDNA cleavage activity. In such embodiments, the CRISPR/Cas effector can be a class II, type V CRISPR/Cas effector, such as a Cas12 effector protein. Exemplary Cas12 effector proteins include Cas12a, Cas12b, Cas12c, Cas12d, Cas12e, C2c4, C2c8, C2c5, C2c10, and C2c9. In preferred embodiments, the Cas12 effector protein is Cas12a (e.g., Lachnospiraceae bacterium ND2006 Cas12a (LbCas12a)) or Cas12b (e.g., Alicyclobacillus acidoterrestris Cas12b (AacCas12b), Alicyclobacillus acidophilus Cas12b (AapCas12b)).

In some embodiments, the CRISPR/Cas effector is an RNA editing enzyme (e.g., RNA endonuclease) with RNA (e.g., ssRNA) cleavage activity. In such embodiments, the CRISPR/Cas effector can be a class II, type VI CRISPR/Cas effector, such as a Cas13 effector protein. Suitable Cas13 effectors include Cas13a/C2c2, Cas13b, Cas13a from Leptotrichia wadeii (LwaCas13a), Leptotrichia shahii (LshCas13a), Cas13b from Prevotella sp. (PspCas13b), Cas13c from Fusobacterium perfoetens (FpeCas13c), and Cas13d from Ruminococcus flavefaciens XPD3002 (aka CasRX). In a preferred embodiment, the Cas13 enzyme is a Cas13a protein derived from thermophilic bacteria, preferably from Thermoclostridium caenicola (TccCas13a) or Herbinix hemicellulosilytica (HheCas13a).

Sequences for HheCas13a are known in the art. See e.g., Addgene plasmid No.: 91871 and East-Seletsky, et al., Mol Cell, 2017, which are hereby incorporated by reference in their entirety. Exemplary encoding DNA and amino acid sequences for HheCas13a are respectively provided below.

### HheCas13a DNA Sequence:

(SEQ ID NO: 94)
ATGAAGTTGACGCGTCGCCGCATCAGTGGCAATTCAGTCGACCAGAAGATT
ACAGCTGCGTTTTATCGTGATATGTCGCAAGGGTTATTATATTATGATTCG
GAGGACAATGACTGTACGGACAAGGTCATCGAAAGTATGGATTTCGAACGC
TCTTGGCGTGGACGCATTCTGAAGAACGGCGAGGATGATAAGAATCCGTTT
TACATGTTCGTTAAAGGTTTAGTTGGTTCCAACGATAAGATCGTGTGCGAA
CCTATCGACGTCGACTCAGACCCTGATAACTTAGATATCTTGATTAACAAA
AATCTGACGGGGTTCGGACGCAACTTGAAAGCACCTGACAGTAACGATACC
TTAGAAAATCTTATCCGCAAAATCCAGGCCGGCATTCCCGAAGAAGAGGTT
CTTCCGGAGCTGAAGAAAATTAAAGAGATGATCCAGAAAGACATCGTTAAC
CGCAAAGAGCAGTTGCTTAAATCCATCAAAAACAACCGTATTCCTTTTTCA
TTGGAAGGTTCCAAGCTGGTCCCTTCCACCAAAAAGATGAAGTGGCTTTTC
AAGTTGATTGACGTTCCTAATAAAACCTTCAATGAAAAAATGTTGGAGAAG
TACTGGGAGATCTACGACTATGACAAGCTTAAGGCGAATATCACGAACCGC
CTGGACAAAACGGATAAGAAGGCACGTTCAATTTCGCGTGCGGTAAGCGAG
GAGTTACGTGAGTACCATAAAAACTTGCGCACAAACTATAATCGCTTCGTA
TCTGGAGACCGTCCAGCAGCCGGGCTTGACAATGGGGGGAGTGCTAAATAT
AATCCCGATAAGGAGGAGTTCTTATTGTTTCTTAAAGAAGTTGAGCAGTAC
TTCAAGAAATACTTCCCAGTCAAGAGCAAGCATTCGAACAAGTCCAAAGAC
AAATCATTGGTTGATAAGTACAAAAATTATTGCAGTTACAAAGTGGTCAAG
AAAGAGGTCAACCGCAGCATCATCAATCAATTGGTGGCTGGACTTATTCAG
CAGGGTAAGCTTCTGTATTACTTCTACTATAACGATACCTGGCAGGAGGAT
TTCCTGAATTCCTACGGTCTTAGTTATATTCAGGTTGAGGAGGCATTTAAA
AAGAGTGTAATGACCAGTCTTTCGTGGGGAATCAACCGTCTGACCTCGTTC
TTTATCGACGACTCGAACACTGTGAAGTTTGATGATATCACAACGAAAAAG
GCTAAGGAGGCGATTGAGTCTAATTATTTCAACAAATTACGCACATGCTCC
CGTATGCAGGACCACTTCAAAGAGAAATTGGCCTTCTTTTACCCGGTGTAT
GTGAAGGACAAGAAGGATCGCCCGGATGATGATATCGAAAACTTAATTGTA
CTGGTTAAGAACGCCATTGAGAGCGTGAGCTATTTGCGCAATCGCACGTTT
CACTTTAAAGAGAGCTCCTTATTGGAGCTGCTTAAAGAGTTAGACGATAAA
AATTCTGGTCAAAATAAAATCGACTATAGCGTAGCCGCTGAATTTATTAAA
CGCGATATCGAGAACTTGTATGACGTCTTCCGTGAACAGATCCGCTCACTT
GGTATTGCTGAGTACTACAAGGCGGACATGATTTCGGACTGTTTTAAGACA
TGCGGGTTAGAATTCGCGCTTTATTCGCCAAAAAATTCGTTGATGCCCGCT
TTTAAGAACGTTTATAAACGCGGAGCAAATCTTAACAAGGCGTATATTCGC
GATAAAGGGCCAAAGGAAACAGGAGACCAGGGTCAGAACTCGTACAAAGCT
TTGGAGGAATATCGTGAGCTTACATGGTATATTGAGGTGAAGAATAACGAC
CAGTCGTACAACGCCTACAAGAATTTGCTTCAACTGATTTATTATCATGCG
TTTCTTCCGGAGGTTCGTGAAAATGAGGCTCTTATCACTGACTTCATTAAC
CGCACTAAAGAGTGGAATCGTAAGGAAACAGAGGAGCGTCTGAACACTAAA
AACAATAAAAAACACAAGAATTTCGACGAGAATGACGATATTACGGTAAAT
ACGTATCGTTACGAATCTATTCCGGATTACCAGGGGGAATCCCTTGATGAT
TATCTTAAGGTGTTACAGCGCAAGCAAATGGCGCGTGCGAAGGAGGTAAAT
GAGAAGGAGGAGGGCAACAATAACTATATCCAATTCATTCGTGACGTCGTA
GTATGGGCGTTCGGGGCCTACTTGGAAAACAAGCTGAAGAATTATAAGAAC
GAACTGCAACCACCGTTGAGCAAAGAGAACATTGGCTTAAATGATACCTTA
AAGGAATTGTTTCCCGAAGAAAAAGTAAAGTCACCATTTAATATCAAGTGC
CGTTTCTCCATTAGTACATTTATCGACAACAAAGGTAAGTCCACCGATAAT
ACCTCGGCCGAGGCCGTCAAGACTGATGGCAAGGAGGACGAAAAGGACAAA
AAGAATATTAAACGCAAAGACTTGCTTTGCTTTTACCTGTTCTTACGCTTG
CTTGATGAAAACGAAATTTGCAAATTACAACACCAATTTATTAAATACCGT
TGCAGTTTAAAAGAGCGTCGTTTTCCAGGTAACCGCACCAAACTGGAGAAG
GAAACTGAACTTTTGGCCGAGCTGGAAGAACTGATGGAACTGGTACGTTTT
ACGATGCCATCAATTCCTGAAATCTCTGCAAAGGCGGAATCAGGTTATGAC
ACAATGATTAAGAAGTATTTTAAAGACTTCATCGAGAAAAAAGTCTTTAAG
AATCCGAAGACGTCCAACCTGTACTACCACTCCGACTCTAAAACTCCGGTC
ACCCGCAAATATATGGCTCTGCTGATGCGTTCTGCGCCTTTGCATCTCTAC
AAGGATATCTTCAAGGGCTACTATTTGATCACTAAGAAGGAGTGTTTGGAG
TATATTAAATTAAGCAACATCATTAAAGACTATCAAAACTCATTGAATGAA
TTGCACGAGCAGCTGGAGCGCATTAAATTAAAGAGTGAGAAACAAAACGGG
AAAGACTCTCTGTATCTTGACAAAAAGGACTTCTATAAAGTAAAGGAGTAC
GTCGAAAATTTAGAACAAGTGGCGTGTTACAAGCACTTGCAGCACAAAATC
AACTTCGAAAGCTTATACCGTATTTTTCGTATTCACGTGGACATCGCTGCG
CGTATGGTCGGTTATACCCAAGACTGGGAGCGTGATATGCACTTTCTTTTC
AAGGCGTTGGTCTATAATGGCGTACTTGAGGAGCGCCGTTTTGAAGCAATC
TTTAACAATAACGATGACAACAACGACGGGCGTATCGTAAAAAAGATCCAG
AATAACTTAAATAACAAGAACCGCGAATTAGTATCTATGTTATGTTGGAAT
AAAAAGCTGAACAAGAACGAATTTGGTGCTATTATTTGGAAGCGTAACCCT
ATCGCCCATCTGAACCACTTTACACAAACGGAACAAAACTCAAAGTCGAGC
CTTGAATCTCTGATCAACTCGCTGCGCATCCTGTTAGCGTACGACCGTAAA
CGTCAAAACGCTGTGACAAAGACTATCAATGACCTTCTGTTAAACGACTAT
CACATCCGCATTAAGTGGGAAGGGCGCGTGGATGAAGGGCAGATCTACTTT
AATATCAAGGAAAAGGAAGACATCGAGAACGAGCCTATTATCCACTTGAAA
CACCTGCATAAAAAGGACTGCTACATTTACAAGAACAGCTATATGTTCGAC
AAGCAAAAGGAGTGGATTTGTAATGGCATTAAAGAAGAAGTTTATGACAAG
TCCATTCTTAAGTGTATCGGAAACTTGTTCAAGTTTGACTATGAGGACAAA
AACAAATCCTCAGCAAATCCTAAGCACACC.

### HheCas13a Amino Acid Sequence:

(SEQ ID NO: 95)
MKLTRRRISGNSVDQKITAAFYRDMSQGLLYYDSEDNDCTDKVIESMDFER
SWRGRILKNGEDDKNPFYMFVKGLVGSNDKIVCEPIDVDSDPDNLDILINK
NLTGFGRNLKAPDSNDTLENLIRKIQAGIPEEEVLPELKKIKEMIQKDIVN
RKEQLLKSIKNNRIPFSLEGSKLVPSTKKMKWLFKLIDVPNKTFNEKMLEK
YWEIYDYDKLKANITNRLDKTDKKARSISRAVSEELREYHKNLRTNYNRFV
SGDRPAAGLDNGGSAKYNPDKEEFLLFLKEVEQYFKKYFPVKSKHSNKSKD
KSLVDKYKNYCSYKVVKKEVNRSIINQLVAGLIQQGKLLYYFYYNDTWQED
FLNSYGLSYIQVEEAFKKSVMTSLSWGINRLTSFFIDDSNTVKFDDITTKK
AKEAIESNYFNKLRTCSRMQDHFKEKLAFFYPVYVKDKKDRPDDDIENLIV
LVKNAIESVSYLRNRTFHFKESSLLELLKELDDKNSGQNKIDYSVAAEFIK
RDIENLYDVFREQIRSLGIAEYYKADMISDCFKTCGLEFALYSPKNSLMPA
FKNVYKRGANLNKAYIRDKGPKETGDQGQNSYKALEEYRELTWYIEVKNND
QSYNAYKNLLQLIYYHAFLPEVRENEALITDFINRTKEWNRKETEERLNTK
NNKKHKNFDENDDITVNTYRYESIPDYQGESLDDYLKVLQRKQMARAKEVN
EKEEGNNNYIQFIRDVVVWAFGAYLENKLKNYKNELQPPLSKENIGLNDTL
KELFPEEKVKSPFNIKCRFSISTFIDNKGKSTDNTSAEAVKTDGKEDEKDK
KNIKRKDLLCFYLFLRLLDENEICKLQHQFIKYRCSLKERRFPGNRTKLEK
ETELLAELEELMELVRFTMPSIPEISAKAESGYDTMIKKYFKDFIEKKVFK
NPKTSNLYYHSDSKTPVTRKYMALLMRSAPLHLYKDIFKGYYLITKKECLE
YIKLSNIIKDYQNSLNELHEQLERIKLKSEKQNGKDSLYLDKKDFYKVKEY
VENLEQVACYKHLQHKINFESLYRIFRIHVDIAARMVGYTQDWERDMHFLF
KALVYNGVLEERRFEAIFNNNDDNNDGRIVKKIQNNLNNKNRELVSMLCWN
KKLNKNEFGAIIWKRNPIAHLNHFTQTEQNSKSSLESLINSLRILLAYDRK
RQNAVTKTINDLLLNDYHIRIKWEGRVDEGQIYFNIKEKEDIENEPIIHLK
HLHKKDCYIYKNSYMFDKQKEWICNGIKEEVYDKSILKCIGNLFKFDYEDK
NKSSANPKHT.

Exemplary encoding DNA and amino acid sequences for TccCas13a are respectively provided below.

### TccCas13a DNA Sequence:

(SEQ ID NO: 69)
ATGAAGATCACGAAAAGGAAATGGGGAGAGCATCATCCGCCGCTTTACTT
CTACCGGGATGAGGACTCCGGCAGGCTCCTGGCACAGAACGACAGAAAGC
AGGATTATACCGATACCCTGTTTAATGATATTGCGCAGGATACATTTGAA
AGATCGCTGAGAAACCGGCTTTTGAAAACACCCGAAAAGGGAGACAAAAG
ATTCTACAGCAACGAGATCGTCAAGCTGGTGGAGAAACTGTGCCAGGGTG
CGGATGTGGCGGAGATCATGAAAAGCATGGAGAGGAACGAAAAGCTGCGC
CCCAAGAATGAAAAAGAGATTAAAAATCTGAAAAAGCAATTGGACGGTAC
CCTTTCCGAATACGGTAAAAGGTATACCGCCCCAGAAGGCGCCATGACCC
TCAACGATGCCTTGTTTTACCTGGTAGAAGGAAACCCTTTAAAGCAGGCC
ATGGCCAAAGCTGAACTGGGCAAAATCCGGGAGGCATTAATAAAGGAAAA
GGAAAACCGCATCAACCGCGTCCGGTATTCCATCAAGAACAATAAAATAC
CGCTGAGAATCCAAGAGGATGGCGGCATTACACCCAATAATGACCGTGCG
GCCTGGCTGCTGGGGCTCATGAAGCCGGCGGACCCGGCAAAAGGAATAAC
CGACTGCTACCCGCTCTTAGGCGAGTTGGAAGAAGTATTCGACTTTGACA
AGCTGTCCAAAACGCTGCACGAAAAGATAAGCCGTTGCCAAGGCCGACCC
CGTTCCATAGCCATGGCGGTCGATGAGGCCCTGAAGCAATATCTCCGGGA
GCTTTGGGAGAAGTCTCCCTCGCGACAGCAGGATCTGAAGTATTACTTTC
AGGCCGTACAGGAGTACTTTAAGGACAATTTTCCCATCCGGACAAAACGG
ATGGGCGCCCGCCTGCGGCAGGAATTGCTCAAGGATAAGACGTCCCTTTC
CCGTCTGCTGGAACCCAAGCATATGGCTAATGCCGTTCGCCGCAGGCTGA
TCAACCAGAGTACCCAGATGCACATCCTTTATGGAAAGCTTTATGCATAT
TGCTGCGGGGAAGACGGCAGGCTTTTGGTAAACAGCGAGACGCTGCAAAG
GATACAGGTCCATGAAGCGGTAAAAAAGCAGGCCATGACGGCCGTGCTGT
GGTCCATATCCCGTCTGCGCTATTTTTACCAGTTTGAAGACGGCGATATC
TTGAGCAATAAAAACCCGATTAAAGATTTCAGAGATAAATTTCTCAGAGA
CACGAATAAATATACCCATGAAGATGTTGAGGCCTGCAAGGAAAAACTGC
AGGACTTCTTCCCGCTGAAAGAATTGCAGGAAAAGATTAAGGAGGATGCA
AAAGGATTACAGGAAACAGACAATAAGCAGGCTGATACAACGGATTTCAA
AGCGATCGGGCACATCGTCAGGGATGATCGGAAGCTCTGCAACCAGTTGC
TGGCGGAGTGCGTTTCCTGCATCGGGGAGCTGAGACATCATATCTTTCAT
TATAAAAATGTGACCTTGATACAGGCGCTCAAAAGGATCGCCGATAAAGT
GAAACCGGAGGATTTGTCTGTGCTCCGGGCCATTTACCTGTTGGACAGGA
GAAACCTCAAAAAGGCGTTTGCCAAAAGAATCAGCAGCATGAACCTTCCG
CTGTATTACAGGGAGGATCTATTATCCCGCATTTTCAAAAAAGAAGGGAC
GGCGTTTTTCCTGTACAGCGCCAAGATCCAGATGACACCGTCCTTTCAGA
GGGTCTATGAGCGTGGTAAAAATCTGCGCCGGGAGTTCGAGTGCGAACGC
ATGAAAGCCGAGGCATCCAACGGACAGAACGGGCAGGATGGCGACCGGCT
GAAATGGTTCCGACAGCTTGCGGCTGGGGATAGCGCCGATACCCATTTCA
ACTGGGCTGTGGAGGCCTATGCGGAATCGGCAGCCGATGTGGAAAACAAC
GTTGAATTCGATACCGATGTGGATGCCCAGCGTGCCCTGCGGAACCTTCT
GCTGCTGATATATAGGCACCATTTCCTGCCGGAGGTGCAGAAGGATGAAA
CCCTGGTGACCGGCAAGATCCATAAGGTTTTGGAAAGAAACAGGCAGCTG
TCTGAAGGCCAAGGGCCAAATCAAGGTAAGGCCCATGGATACAGCGTTAT
AGAAGAGCTGTATCATGAAGGCATGCCGCTTTCCGATCTCATGAAGCAAT
TGCAGCGGAGGATCTCCGAAACCGAAAGGGAGAGCCGGGAACTGGCACAG
GAAAAAACGGATTATGCCCAGCGCTTTATCCTCGATATCTTTGCCGAGGC
CTTTAACGATTTCCTGGAGGCGCACTATGGCGAAGAATACCTTGAGATCA
TGAGCCCCAGGAAAGATGCCGAAGCGGCGAAGAAATGGGTAAAAGAAAGC
AAAACGGTGGATTTGAAAACATCTATAGACGAAAAAGAACCGGAAGGGCA
TCTCCTGGTGCTCTATCCCGTCCTGCGCCTGCTGGACGAAAGGGAACTGG
GAGAGCTTCAGCAGCAGATGATCCGTTATCGGACATCCCTGGCCAGTTGG
CAGGGTGAGAGCAATTTCAGTGAAGAAATAAGGATAGCCGGGCAGATTGA
GGAATTGACCGAACTGGTCAAGCTGACGGAACCGGAGCCCCAGTTTGCGG
AGGAAGTATGGGGGAAACGGGCTAAAGAAGCGTTTGAAGACTTTATTGAA
GGAAACATGAAAAATTATGAGGCTTTTTATCTTCAGAGCGACAACAACAC
GCCGGTATATCGGAGGAACATGAGCCGGTTGCTGCGCTCGGGGCTTATGG
GAGTGTACCAAAAGGTGCTTGCCAGCCACAAGCAGGCGCTCAAAAGAGAT
TACTTGCTCTGGTCGGAAAAACATTGGAACGTGAAGGATGAGAATGGAGC
GGATATCTCTTCCGCTGAACAGGCCCAATGCCTTTTGCAGCGGCTCCATA
GGAAGTACGCCGAATCCCCGTCCCGATTTACGGAGGAGGACTGCAAACTG
TATGAGAAGGTTCTCCGGCGGCTTGAGGACTATAACCAGGCCGTGAAAAA
CCTGTCCTTCAGTTCATTGTATGAAATATGCGTCCTCAATCTTGAAATCC
TCTCCCGATGGGTGGGGTTTGTCCAGGACTGGGAGCGGGATATGTACTTC
CTCCTTTTGGCGTGGGTCAGACAGGGCAAACTGGACGGGATAAAGGAGGA
AGATGTCAGAGATATCTTCTCTGAAGGCAACATTATTCGCAATCTGGTGG
ATACGCTGAAAGGCGAAAACATGAATGCCTTTGAAAGCGTTTATTTCCCT
GAGAATAAGGGGTCTAAGTATTTAGGTGTGCGGAATGATGTTGCCCATCT
GGATCTGATGCGGAAAAACGGATGGCGGTTGGAGGCTGGCAAAACCTGCA
GCGTGATGGAAGATTACATCAACCGTTTGAGGTTTCTCCTGTCCTATGAC
CAGAAACGCATGAACGCCGTCACCAAGACCTTGCAGCAGATATTTGATAG
GCATAAAGTCAAAATCCGGTTCACCGTGGAGAAAGGAGGAATGCTGAAGA
TAGAGGATGTGACTGCCGACAAGATCGTGCATCTGAAAGGTTCCAGGTTG
AGCGGCATTGAAATTCCAAGTCACGGGGAGAGGTTTATTGACACGTTGAA
AGCGCTGATGGTATACCCGAGAGGATGA.

### TccCas13a Amino Acid Sequence:

(SEQ ID NO: 67)
MKITKRKWGEHHPPLYFYRDEDSGRLLAQNDRKQDYTDTLFNDIAQDTFE
RSLRNRLLKTPEKGDKRFYSNEIVKLVEKLCQGADVAEIMKSMERNEKLR
PKNEKEIKNLKKQLDGTLSEYGKRYTAPEGAMTLNDALFYLVEGNPLKQA
AKAELGKIREALIKEKENRINRVRYSIKNNKIPLRIQEDGGITPNNDRAA
WLLGLMKPADPAKGITDCYPLLGELEEVFDFDKLSKTLHEKISRCQGRPR
SIAMAVDEALKQYLRELWEKSPSRQQDLKYYFQAVQEYFKDNFPIRTKRM
GARLRQELLKDKTSLSRLLEPKHMANAVRRRLINQSTQMHILYGKLYAYC
CGEDGRLLVNSETLQRIQVHEAVKKQAMTAVLWSISRLRYFYQFEDGDIL
SNKNPIKDFRDKFLRDTNKYTHEDVEACKEKLQDFFPLKELQEKIKEDAK
GLQETDNKQADTTDFKAIGHIVRDDRKLCNQLLAECVSCIGELRHHIFHY
KNVTLIQALKRIADKVKPEDLSVLRAIYLLDRRNLKKAFAKRISSMNLPL
YYREDLLSRIFKKEGTAFFLYSAKIQMTPSFQRVYERGKNLRREFECERM
KAEASNGQNGQDGDRLKWFRQLAAGDSADTHFNWAVEAYAESAADVENNV
EFDTDVDAQRALRNLLLLIYRHHFLPEVQKDETLVTGKIHKVLERNRQLS
EGQGPNQGKAHGYSVIEELYHEGMPLSDLMKQLQRRISETERESRELAQE
KTDYAQRFILDIFAEAFNDFLEAHYGEEYLEIMSPRKDAEAAKKWVKESK
TVDLKTSIDEKEPEGHLLVLYPVLRLLDERELGELQQQMIRYRTSLASWQ
GESNFSEEIRIAGQIEELTELVKLTEPEPQFAEEVWGKRAKEAFEDFIEG
NMKNYEAFYLQSDNNTPVYRRNMSRLLRSGLMGVYQKVLASHKQALKRDY
LLWSEKHWNVKDENGADISSAEQAQCLLQRLHRKYAESPSRFTEEDCKLY
EKVLRRLEDYNQAVKNLSFSSLYEICVLNLEILSRWVGFVQDWERDMYFL
LLAWVRQGKLDGIKEEDVRDIFSEGNIIRNLVDTLKGENMNAFESVYFPE
NKGSKYLGVRNDVAHLDLMRKNGWRLEAGKTCSVMEDYINRLRFLLSYDQ
KRMNAVTKTLQQIFDRHKVKIRFTVEKGGMLKIEDVTADKIVHLKGSRLS
GIEIPSHGERFIDTLKALMVYPRG.

In another preferred embodiment, the Cas13 enzyme is a Cas13 protein derived from a bacterium in the Proteobacteria phylum. An exemplary Proteobacteria-derived Cas13 is mCas13 having the following amino acid sequence:

MGIDYSLTSDCYRGINKSCFAVALNIAYDNCDHKGCRTLLSEVLRSKGGI
SDEQIKSQVVDGIQKRLKDIRNYFSHYYHAEDCLRFGDQDAVKVFLEEIY
KNAESKTVGATKESDYKGVVPPLFELHNGTYMITAAGVIFLASFFCHRSN
VYRMLGAVKGFKHTGKEQLSDGQKRDYGFTRRLLAYYALRDSYSVGAEDK
TRCFREILSYLSRVPQLAVDWLNEQQLLTPEEKEAFLNQPAEDEGGDISD
SSSSDKNKKSKEKRRSLRRDEKFILFAIQFIEGWAAEQGLDVTFARYQKT
VEKAENKNQDGKQARAVQLKYRNQGLNPDFNNEWMYYIQNEHAIIQIKLN
NKKAVAARISENELKYLVLLIFEEKGNDAVQKLNCYIYSMSQKIEGEWKH
RPEDERWMPSFTKRADRTVTPEAVQSRLSYIRKQLQETIEKIGQEEPRNN
KWLIYKGKKISMILKFISDSIRDIQRRPNVKQYHILRDALQRLDFDGFYK
ELQNYVNDGRIAVSLYDQIKGVNDISGLCKKVCELTLERLAGLEAKNGSE
LRRYIGLEAQEKHPKYGEWNTLQEKAKRFLESQFSIGKNFLRKMFYGDCC
QKRCFDEEKGYNTQAKERKSLYSIVKEKLKDIKPIHDDRWYLIDRNPKNY
DNKHSRIIRQMCNTYIQDVLCMKMAMWHYEKLISATEFRNKLEWNCIGQG
NMGYERYSLWYKTGCGVVIQFTPADFLRLDIIEKPAMIENICQCFVLGNK
KLNSGAEKKITWDKFNKDGIAKYRKRQAEAVRAIFAFEEGLKIQEDKWSH
ERYFPFCNILDEAVKQGKIKDTGKDKEALNRGRNDFFHEEFKSTEDQQAI
FQKYFPIVERKDDTKKRRDKKQK (SEQ ID NO: 68; GenBank
accession No.: HFH51004.1).

In some embodiments, the Proteobacteria-derived Cas13 is mCas13 encoded by the following DNA sequence:

ATGGGTATTGATTATTCGCTTACAAGTGACTGTTATCGAGGCATCAACAA
GTCTTGTTTTGCAGTTGCTTTGAATATTGCATATGATAACTGTGATCATA
AAGGATGTAGGACTCTTCTGAGTGAGGTGCTGCGCAGCAAGGGAGGGATT
TCTGATGAGCAAATAAAATCACAAGTAGTGGATGGAATTCAGAAGCGACT
TAAAGACATTCGCAATTATTTCTCGCATTATTACCACGCAGAAGACTGTC
TGCGGTTTGGAGACCAAGATGCCGTCAAGGTTTTTCTGGAAGAAATATAC
AAGAACGCGGAATCAAAGACTGTCGGAGCGACAAAAGAAAGCGACTATAA
AGGCGTTGTGCCGCCATTATTTGAATTGCATAACGGTACATATATGATTA
CGGCGGCGGGGGTTATTTTTCTGGCATCATTTTTCTGCCATCGGAGTAAT
GTCTATCGGATGCTGGGAGCGGTGAAAGGATTTAAACATACCGGAAAAGA
GCAATTGAGCGATGGGCAGAAACGAGATTATGGTTTCACTCGCCGGCTGC
TGGCTTATTATGCGCTCCGGGACAGCTATTCTGTGGGGGCGGAAGACAAG
ACACGATGTTTCCGCGAGATATTAAGCTATTTGTCGAGAGTACCGCAATT
GGCAGTAGATTGGCTGAATGAACAACAGCTGCTTACACCAGAAGAAAAAG
AAGCTTTTTTGAATCAACCCGCCGAAGATGAGGGCGGGGATATTTCGGAC
TCTTCGTCCAGTGATAAAAATAAAAAAAGCAAAGAAAAGAGGCGTAGTCT
CCGCAGAGATGAAAAATTCATTTTATTTGCTATCCAATTTATTGAGGGGT
GGGCGGCTGAACAGGGATTAGATGTGACATTTGCACGCTACCAAAAAACA
GTGGAAAAAGCGGAGAATAAGAATCAGGATGGCAAACAGGCCAGAGCGGT
GCAATTAAAATACAGAAACCAAGGACTCAATCCGGATTTCAATAACGAAT
GGATGTATTACATTCAGAATGAACACGCAATTATCCAGATTAAACTGAAT
AATAAAAAAGCCGTTGCTGCCCGTATTTCTGAAAATGAATTAAAATATCT
GGTGCTGCTGATTTTTGAGGAGAAGGGCAATGACGCCGTCCAAAAGCTGA
ATTGTTATATTTACAGTATGAGCCAAAAAATCGAGGGCGAATGGAAACAC
AGGCCGGAGGATGAGCGATGGATGCCGTCGTTTACCAAGCGTGCCGACAG
GACGGTTACGCCGGAGGCAGTGCAGAGCCGGTTAAGCTATATTCGCAAAC
AACTTCAGGAGACGATAGAGAAAATCGGTCAGGAAGAGCCGCGGAATAAT
AAGTGGCTGATATACAAAGGCAAAAAAATATCAATGATACTGAAGTTTAT
CTCCGACAGCATTCGCGATATTCAAAGGCGGCCGAATGTGAAACAATATC
ATATTTTACGCGATGCGCTTCAGAGGCTCGACTTTGATGGATTTTATAAG
GAACTTCAAAATTACGTCAATGACGGCCGGATTGCGGTTTCATTATACGA
TCAGATCAAGGGTGTCAATGACATCAGCGGGCTTTGTAAAAAAGTCTGCG
AACTGACACTTGAAAGACTGGCTGGGCTGGAGGCAAAGAATGGCTCCGAG
CTGAGGCGTTATATTGGGCTTGAAGCGCAGGAAAAACATCCGAAGTACGG
AGAGTGGAACACACTGCAGGAAAAGGCCAAGCGATTTCTGGAGTCGCAGT
TTTCTATCGGGAAAAACTTTTTGCGGAAAATGTTTTATGGCGATTGTTGT
CAAAAGCGGTGCTTTGACGAAGAAAAAGGTTACAATACACAAGCGAAAGA
GCGAAAAAGTCTGTATAGCATTGTGAAAGAAAAACTCAAGGACATCAAGC
CCATACACGATGACCGGTGGTATCTTATCGACAGGAATCCGAAGAACTAT
GACAATAAACACAGCAGGATTATCCGACAGATGTGCAACACCTATATACA
AGATGTTCTCTGTATGAAGATGGCGATGTGGCATTATGAGAAATTAATCT
CCGCGACTGAATTCAGGAATAAATTGGAATGGAATTGTATTGGGCAGGGA
AATATGGGTTATGAGCGATATTCGCTGTGGTATAAGACAGGCTGCGGGGT
TGTCATACAATTTACGCCGGCGGATTTTTTGCGACTGGATATCATTGAAA
AACCTGCGATGATAGAAAATATTTGCCAGTGTTTTGTGCTCGGAAACAAA
AAACTCAATTCGGGTGCCGAAAAGAAGATAACCTGGGATAAATTCAATAA
AGACGGCATTGCAAAATACAGAAAGCGGCAGGCCGAGGCTGTGCGTGCGA
TATTTGCGTTTGAGGAAGGTTTAAAAATACAAGAGGATAAATGGAGTCAT
GAGAGATACTTTCCATTTTGCAATATTCTTGACGAGGCGGTAAAACAAGG
TAAAATAAAAGATACAGGCAAAGACAAGGAGGCTCTGAATCGCGGCAGAA
ATGATTTTTTTCATGAGGAATTTAAGTCAACAGAAGATCAGCAGGCGATT
TTTCAGAAATACTTTCCGATTGTCGAACGAAAAGACGACACAAAAAAACG
GCGAGATAAAAAGCAAAAGTGA (SEQ ID NO: 96; GenBank
accession No.: DSVK01000191.1).

Also disclosed are Cas effector protein variants including one or more mutations (e.g., conservative or non-conservative mutations) relative to any of the Cas effector proteins disclosed herein. For example, it is also contemplated that other Cas13 variants can be evolved from those disclosed herein, for example, by targeted mutation of one or more amino acid residues in specific regions of the enzyme. Such mutation(s) may alter substrate binding, alter conformation of bound substrate, alter substrate accessibility to the active site, alter tolerance to non-optimal presentation of a target sequence to the active site, and/or alter target sequence specificity (recognition). In some embodiments, a suitable Cas13 effector protein has an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in any one of SEQ ID NOs:67, 68 or 95.

In some embodiments, a suitable Cas13 has at least 70%, at least 75%, at least 80%, at least 85%, at least 90%, at least 95%, at least 96%, at least 97%, at least 98%, or at least 99% sequence identity with the sequences of any of the SEQ ID numbers or Uniprot accession numbers disclosed herein, such as SEQ ID NOs:67, 68, and 95, and including nucleic acid sequences encoding amino acid sequences thereof (e.g., SEQ ID NOs:69, 94 and 96).

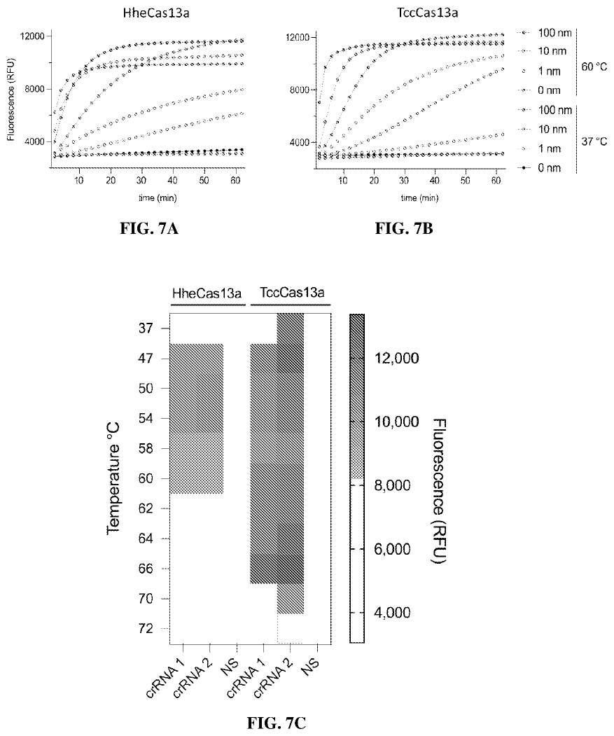

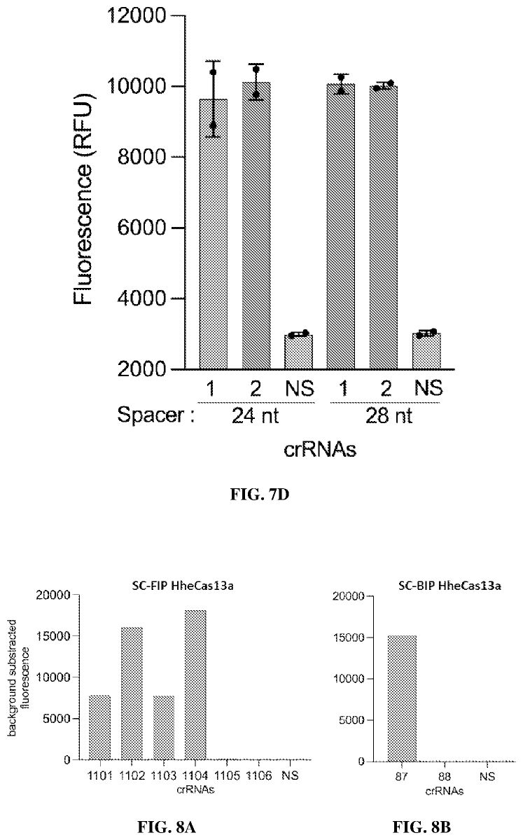

Preferably, the Cas12 and/or Cas13 proteins are thermostable enzymes. The working examples demonstrate that HheCas13a exhibits robust activity at temperatures up to 60° C. while TccCas13a maintains a robust activity at temperatures as high as −70° C. (see FIG. 7C). Thus, in some embodiments, the Cas13 enzyme exhibits ssRNA cleavage activity at a temperature of about 37-70° C., preferably about 47-60° C., more preferably about 60° C.

Typically, the Cas12- or Cas13-based RNP complex can be formed by contacting or incubating the Cas effector protein with the appropriate crRNA. Preferably, the crRNA is designed to be complementary to the RT-LAMP amplification product or the RNA transcript derived therefrom. In preferred embodiments, the crRNA targets regions of the SARS-CoV-2 N or E genes.

Exemplary crRNAs that can be included in the Cas12- or Cas13-based RNP complex are crRNAs encoded by the nucleic acid sequence of SEQ ID NOs:5-8, 61 and 72-92, sequences having at least 70%, 75%, 80%, 85%, 90%, 95%, 96%, 97%, 98%, or 99% sequence identity thereto, nucleic acid sequences having 1, 2, 3, 4, 5, 6, 7, 8, 9, or 10 nucleic acid substitution(s), addition(s), deletion(s), or a combination thereof relative thereto, or the reverse complement of any of the foregoing. In some embodiments, a crRNA encoded by the nucleic acid sequence of SEQ ID NO:61, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s) is preferably used with TccCas13a or HheCas13a. In some embodiments, a crRNA encoded by the nucleic acid sequence of SEQ ID NO:75, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s) are preferably used with mCas13.

F. Activatable Oligonucleotide Reporter

Also provided are activatable ssDNA or ssRNA oligonucleotide reporters that can be non-specifically cleaved by the appropriate CRISPR/Cas effector protein in the presence of the specific/target amplicon. Cleavage of the ssDNA or ssRNA oligonucleotide can be used as an indicator of the presence or absence of the specific/targeted SARS-CoV-2 derived-amplicon.

The activatable ssDNA or ssRNA oligonucleotide can include any appropriate nucleotide sequence to which the CRISPR/Cas effector protein exhibits collateral cleavage activity. As demonstrated in the working examples, suitable activatable ssDNA oligonucleotides include, without limitation, poly T oligonucleotides such as TTTTT, TTATT and TTATTATT. Suitable activatable ssRNA oligonucleotides include, without limitation, UGACGU, UUUUU, and UUUUUU.

In preferred embodiments, the activatable oligonucleotide is a labeled single stranded detector DNA (detector ssDNA) that includes a fluorescence-emitting dye pair; the Type V CRISPR/Cas effector protein (e.g., a Cas12 protein such as Cas12a, Cas12b, Cas12c, Cas12d, Cas12e) cleaves the labeled detector ssDNA after it is activated (by the Cas12/crRNA RNP complex hybridizing to the target DNA, herein the RT-LAMP product); and the detectable signal that is measured is produced by the fluorescence-emitting dye pair. In some preferred embodiments, the activatable oligonucleotide is a labeled single stranded detector RNA (detector ssRNA) that includes a fluorescence-emitting dye pair; the Type VI CRISPR/Cas effector protein (e.g., a Cas13 protein such as mCas13, TccCas13a, HheCas13a) cleaves the labeled detector ssRNA after it is activated (by the Cas13/crRNA RNP complex hybridizing to the target RNA transcript generated from the RT-LAMP product); and the detectable signal that is measured is produced by the fluorescence-emitting dye pair.

The labelled activatable oligonucleotide can be a ssDNA or ssRNA containing a fluor/quencher pair or a FRET pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

The activatable ssDNA/ssRNA preferably includes a quencher/fluor pair. An amount of detectable signal increases when the labeled detector ssDNA/ssRNA is cleaved. For example, in some cases, the signal exhibited by one signal partner (a signal moiety) is quenched by the other signal partner (a quencher signal moiety), e.g., when both are present on the same ssDNA/ssRNA molecule prior to cleavage by a Type V or VI CRISPR/Cas effector protein (e.g., a Cas12 protein such as Cas12a, Cas12b, Cas12c, Cas12d, Cas12e or a Cas13 protein such as mCas13, TccCas13a, HheCas13a). Such a signal pair is referred to herein as a “quencher/fluor pair”, “quenching pair”, or “signal quenching pair.” For example, in some cases, one signal partner (e.g., the first signal partner) is a signal moiety that produces a detectable signal that is quenched by the second signal partner (e.g., a quencher moiety). The signal partners of such a quencher/fluor pair will thus produce a detectable signal when the partners are separated (e.g., after cleavage of the detector ssDNA/ssRNA by a Type V or VI CRISPR/Cas effector protein), but the signal will be quenched when the partners are in close proximity (e.g., prior to cleavage of the detector ssDNA/ssRNA by a Type V or VI CRISPR/Cas effector protein (e.g., a Cas12 protein such as Cas12a, Cas12b, Cas12c, Cas12d, Cas12e or a Cas13 protein such as mCas13, TccCas13a, HheCas13a)).

A quencher moiety can quench a signal from the signal moiety (e.g., prior to cleave of the detector ssDNA/ssRNA by a Type VI CRISPR/Cas effector protein (e.g., a Cas12 protein such as Cas12a, Cas12b, Cas12c, Cas12d, Cas12e or a Cas13 protein such as mCas13, TccCas13a, HheCas13a)) to various degrees. In some forms, a quencher moiety quenches the signal from the signal moiety where the signal detected in the presence of the quencher moiety (when the signal partners are in proximity to one another) is 95% or less of the signal detected in the absence of the quencher moiety (when the signal partners are separated). For example, in some cases, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. In some cases, no signal (e.g., above background) is detected in the presence of the quencher moiety. The signal moiety is preferably a fluorescent label. The quencher moiety quenches the signal (the light signal) from the fluorescent label (e.g., by absorbing energy in the emission spectra of the label). Thus, when the quencher moiety is not in proximity with the signal moiety, the emission (the signal) from the fluorescent label is detectable because the signal is not absorbed by the quencher moiety.

Any convenient donor acceptor pair (signal moiety/quencher moiety pair) can be used and many suitable pairs are known in the art. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety. In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange. Examples of AlexaFluor dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like. Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some forms (e.g., when the detector ssDNA/ssRNA includes a FRET pair) the labeled activatable ssDNA/ssRNA oligonucleotide produces an amount of detectable signal prior to being cleaved, and the amount of detectable signal that is measured is reduced when the labeled ssDNA/ssRNA is cleaved. In some forms, the labeled ssDNA/ssRNA produces a first detectable signal prior to being cleaved (e.g., from a FRET pair) and a second detectable signal when the labeled detector ssDNA/ssRNA is cleaved (e.g., from a quencher/fluor pair).

FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Förster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”. The donor-acceptor pair (a FRET donor moiety and a FRET acceptor moiety) is referred to herein as a “FRET pair” or a “signal FRET pair.” Thus, in some cases, a subject labeled ssDNA/ssRNA includes two signal partners (a signal pair), when one signal partner is a FRET donor moiety and the other signal partner is a FRET acceptor moiety. A subject labeled detector ssDNA/ssRNA that includes such a FRET pair (a FRET donor moiety and a FRET acceptor moiety) will thus exhibit a detectable signal (a FRET signal) when the signal partners are in close proximity (e.g., while on the same DNA/RNA molecule), but the signal will be reduced (or absent) when the partners are separated (e.g., after cleavage of the DNA/RNA molecule by a Type V or VI CRISPR/Cas effector protein (e.g., a Cas12 protein such as Cas12a, Cas12b, Cas12c, Cas12d, Cas12e or a Cas13 protein such as mCas13, TccCas13a, HheCas13a)).

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in the following table.

Examples of Fret pairs (donor and acceptor FRET moieties)

| Donor | Acceptor |
|---|---|
| Tryptophan | Dansyl |
| 5-(2- | N-4-dimethylamino-3,5- |
| iodoacetylaminoathly)aminonaphthalene- | dinitropheyl)maleimide |
| 1-sulfonic acid | |
| BFP | DsRFP |
| Dansyl | Fluorescein |
| | Isothiocyanate (FITC) |
| Dansyl | Octadecylrhodamine |
| Cyan Flurescent protein (CFP) | Green Fluorescent protein (GFP) |
| Carboxyfluorescein succinimidyl ester | Texas red |
| Cy3 | Cy5 |
| Rhodamine 110 | Cy3 |
| Rhodamine 6G | Malchite green |

In some embodiments, the activatable ssDNA/ssRNA oligonucleotides includes hexachloro-fluorescein (HEX) moiety, FAM moiety, biotin moiety, or a combination thereof.

G. Buffers

The disclosed compositions preferably include buffers. For example, the primers and DNA polymerase can be provided with additional reagents needed for an RT-LAMP assay, including, but not limited to, buffers, and dNTPs. In some embodiments, the one or more primers and/or DNA polymerase are provided in a buffer suitable for RT-LAMP.

Typically, the buffers provide appropriate pH and ionic conditions for the one or more enzymes. For example, a buffer can be an aqueous solution that provides optimal pH, ionic strength, cofactors, and the like for optimal enzyme activity. In some embodiments, the buffers are suitable for storage of the enzymes. In preferred embodiments, the buffers are suitable for RT-LAMP (e.g., the RT and PCR reactions can be performed in the same buffer). The buffers can help relieve RT-mediated inhibition of DNA polymerase activity. In preferred embodiments, the buffers are suitable for RNA transcription and/or CRISPR/Cas based detection (e.g., one-pot transcription and CRISPR/Cas based detection). In preferred embodiments, the buffers are suitable for both RT-LAMP and CRISPR/Cas based detection (e.g., one-pot RT-LAMP coupled with CRISPR/Cas based detection). In preferred embodiments, the buffers are suitable for RT-LAMP, transcription and CRISPR/Cas based detection (e.g., one-pot RT-LAMP coupled with transcription and CRISPR/Cas based detection).

Suitable components of the buffers include, without limitation, one or more salts, reducing agents, buffering agents, deoxynucleoside triphosphates (dNTPs), or combinations thereof. The one or more salts provide monovalent or divalent cations, such as, Mg2+, Mn2+, K+, NH4+, and Na+. Exemplary salts that can be included in the buffers are KCl, MgCl2, NaCl, MnCl₂, NH4Cl, MgSO4, (NH4)₂SO₄, and magnesium acetate (e.g., Mg(C2H302)₂; Mg(CH3COO)₂·4H₂O).

The nucleotide components (dNTPs) serve as the “building blocks” for newly synthesized nucleic acids, being incorporated therein by the action of the reverse transcriptases or DNA/RNA polymerases. Examples of nucleotides suitable for use in the compositions include, but are not limited to, dUTP, dATP, dTTP, dCTP, dGTP, dITP, 7-deaza-dGTP, α-thio-dATP, α-thio-dTTP, α-thio-dGTP, α-thio-dCTP or derivatives thereof, all of which are available commercially from sources including Life Technologies, Inc. (Rockville, Md.), New England BioLabs (Beverly, Mass.) and Sigma Chemical Company (Saint Louis, Mo.). In preferred embodiments, the following dNTPs are included in the compositions: dATP, dTTP, dUTP, dGTP, and dCTP.

The buffer may also include additional components, such as detergents (e.g., TRITON-X100), or other additives (such as betaine or dimethylsulfoxide). One of skill in the art can select an appropriate buffer and any additives.

H. Samples

Appropriate samples include any conventional biological samples, including clinical samples obtained from a human or veterinary subject. Suitable samples include all biological samples useful for detection of infection in subjects, including, but not limited to, cells (such as buccal cells or peripheral blood mononuclear cells), tissues, autopsy samples, bone marrow aspirates, bodily fluids (for example, blood, serum, plasma, urine, cerebrospinal fluid, middle ear fluids, bronchoalveolar lavage, tracheal aspirates, sputum, nasopharyngeal aspirates, oropharyngeal aspirates, or saliva), oral swabs, eye swabs, cervical swabs, vaginal swabs, rectal swabs, stool, and stool suspensions.

In preferred embodiments, the sample is mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, sputa/deep throat saliva, feces, mucosal excretions, plasma, serum, or whole blood. In some embodiments, the sample is a nucleic acid isolated and/or derived from any of the foregoing biological samples.

Generally, the sample is obtained non-invasively, such as by swabbing, scraping, collecting, drawing, or draining

The sample can be used directly or can be processed, such as by adding solvents, preservatives, buffers, or other compounds or substances. In some examples, nucleic acids are isolated and/or derived from the sample. In other examples, isolation of nucleic acids from the sample is not necessary prior to use and the sample (such as a plasma or serum sample) is used directly (without nucleic acid extraction, but potentially with heat-treatment or other processing step). In some embodiments, the sample can be pre-treated with a lysis buffer.

Samples also include isolated nucleic acids, such as DNA or RNA isolated from a biological specimen from a subject, a viral isolate, or other source of nucleic acids. The sample can also include DNA that is reverse transcribed from RNA isolated or extracted from a biological specimen from a subject, a viral isolate, or other source of nucleic acids. Methods for extracting nucleic acids such as RNA or DNA from a sample are known to one of skill in the art. Such methods will depend upon, for example, the type of sample in which the nucleic acid is found. Nucleic acids can be extracted using standard methods. For instance, rapid nucleic acid preparation can be performed using commercially available reagents/kit, such as kits and/or instruments from Invitrogen (TRIzol) Zymo Research (Direct-Zol RNA Miniprep kit), Qiagen (such as QiaAmpO, DNEasy® or RNEasy® kits), Roche Applied Science (such as MagNA Pure kits and instruments), Thermo Scientific (KingFisher mL), bioMerieux (Nuclisens® NASBA Diagnostics), or Epicentre (Masterpure™ kits). In some embodiments, the nucleic acids may be extracted using guanidinium isothiocyanate, such as single-step isolation by acid guanidinium isothiocyanate-phenol-chloroform extraction (Chomczynski et al. Anal. Biochem. 162:156-159, 1987).

### III. Methods of Use

The methods may be used for any purpose for which detection of viral, bacterial or other nucleic acids is desirable, including diagnostic and prognostic applications, such as in laboratory and clinical settings. In some embodiments, the methods may be used for detection of a nucleic acid for genotyping.

In some embodiments, the nucleic acid to be detected is diagnostic for a disease state. The disease state can be an infection, an organ disease, a blood disease, an immune system disease, a cancer, a brain and nervous system disease, an endocrine disease, a pregnancy or childbirth-related disease, an inherited disease, or an environmentally-acquired disease, cancer, or a fungal infection, a bacterial infection, a parasite infection, or a viral infection. Thus, in some embodiments, the method is useful for detecting a nucleic acid (e.g., DNA or RNA) from a bacterium, fungus, virus (e.g., caused by a double-stranded RNA virus, a positive sense RNA virus, a negative sense RNA virus, a retrovirus, etc.), or parasite.

Exemplary viruses that can be detected include, without limitation, Myoviridae, Podoviridae, Siphoviridae, Alloherpesviridae, Herpesviridae (including human herpes virus, and Varicella Zorter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In some embodiments, the virus is coronavirus (e.g., SARS-Cov-2), SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In some embodiments, the nucleic acid to be detected can be associated with a pathogen, including pathogenic bacteria such as, E. faecalis, E. faecium Listeria monocytogenes, Campylobacter jejuni, Staphylococcus aureus (e.g., MRSA), E. coli O157:H7, Borrelia burgdorferi, Helicobacter pylori, Ehrlichia chaffeensis, Clostridium difficil, Vibrio cholerae O139, Salmonella enterica, Bartonella henselae, Streptococcus pyogenes, Chlamydia pneumoniae, Clostridium botulinum, Corynebacterium amycolatum, Klebsiella pneumoniae Vibrio vulnificus, and Parachlamydia.

The disclosed methods typically involve a combination of RT-LAMP and CRISPR-Cas-mediated reactions, optionally with an intervening RNA polymerase-mediated transcription reaction.

Typically, the methods involve RT-LAMP for the isothermal amplification of a target nucleic acid, such as SARS-CoV-2 RNA. Then, the methods employ collateral cleavage of a single-stranded reporter oligonucleotide by a CRISPR/Cas effector protein such as Cas12 or Cas13. The single-stranded reporter oligonucleotide is only cleaved when a RNP complex composed of the Cas effector protein and an associated sgRNA/crRNA recognizes, binds, and/or cleaves the specifically targeted nucleic acid amplified from the first step. Thus, the recognition, binding and/or cleavage of the specifically targeted nucleic acid “activates” the Cas effector protein's collateral cleavage activity toward the single-stranded reporter oligonucleotide. Cleavage of the reporter oligonucleotide produces a signal that can be detected by any appropriate method, such as fluorescence.

In some embodiments, the RT-LAMP product is transcribed to generate an RNA transcript that is then subjected to the CRISPR/Cas cleavage step. For example, Cas13 cleaves ssRNA; thus preferably, an RNA transcript generated from the RT-LAMP product is subjected to the Cas-13 RNP cleavage step.

The disclosed RT-LAMP CRISPR/Cas12 or Cas13 coupled detection assays are highly sensitive. Preferably, the methods are capable of detecting low copies of a target nucleic acid, such as SARS-CoV-2 nucleic acid. For example, in some embodiments, the limit of detection is about 2-20 copies/μL of the target nucleic acid (SARS-CoV-2 RNA), for example about 4-8 copies/μL or 5-10 copies/μL. In some embodiments, the limit of detection is about 1, 2, 3, 4, 5, 6, 7, 8, 9, 10, 11, 12, 13, 14, 15, 16, 17, 18, 19, or 20 copies/μL.

In some embodiments, the RT-LAMP CRISPR/Cas12 or Cas13 coupled detection method is performed as a one-pot assay (e.g., when the enzymes used have compatible optimal temperatures). In some embodiments, the RT-LAMP CRISPR/Cas12 or Cas13 coupled detection method is performed as a two-pot assay (e.g., when the Cas enzyme is substantially less active or inactive at the relatively high temperature used for the RT-LAMP step).

In some embodiments, the iSCAN detection assays include two steps. In the first step, RT-LAMP is performed to reverse transcribe and pre-amplify the viral RNA to generate dsDNA substrates for Cas12 enzymes. In the second step, the RT-LAMP product is used as a target in a reaction with the Cas12 enzyme and a molecular beacon/activatable DNA probe. The crRNAs used with the Cas12 enzyme are designed to be specific to the SARS-CoV-2 N and E gene genomic regions amplified with the RT-LAMP primer sets, and will hybridize to the RT-LAMP product. Binding of the Cas12/crRNA RNP complex to its complimentary RT-LAMP product activates cleavage of an activatable oligonucleotide/probe/molecular beacon (that does not hybridize with the crRNA introduced into the assay, the detection of which confirms the presence of the RT-LAMP product in the sample, and hence the presence of SARS-CoV-2 in the sample. In a preferred embodiment, the N gene of SARS-CoV-2 is used as the target in the disclosed methods.

In one embodiment, the iSCAN detection method is a one pot assay, meaning all assay reagents required for RT-LAMP and Cas12 detection are combined in one tube/container. In other embodiments, the reaction is conducted as a two pot assay, meaning, two separate reaction tubes/containers are used, such as the RT-LAMP reaction being performed in a first tube and the Cas12 based detection being performed in a second tube.

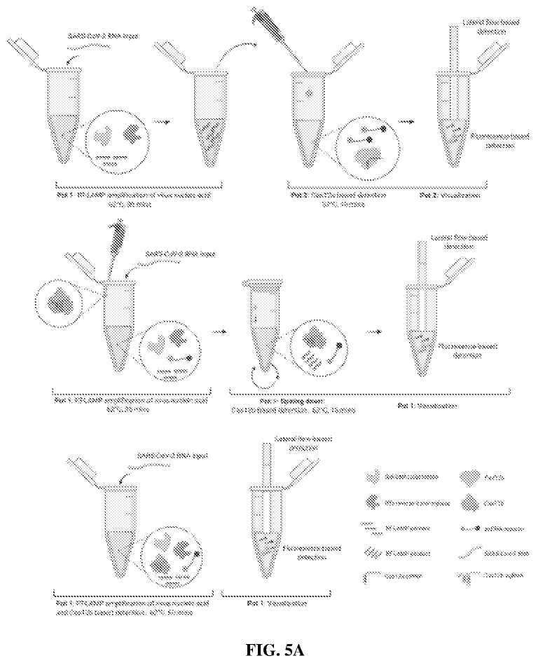

FIG. 5A illustrates the one-pot and two-pot assays coupling RT-LAMP with Cas12-based detection.

A. RT-LAMP

As introduced above, generally, RT-LAMP uses specially designed primers to recognize distinct target sequences on the template strand. Two of the required primers are “inner primers” (FIP and BIP), designed to synthesize new DNA strands. Two outer primers (F3 and B3) anneal to the template strand and also generate new DNA strands. These primers are accompanied by DNA polymerase, which aids in strand displacement and releases the newly formed DNA strands. The BIP primer, accompanied by reverse transcriptase, initiates the process by binding to a target sequence on the 3′ end of the RNA template and synthesizing a copy DNA strand. The B3 primer binds to this side of the template strand as well, and with the help of DNA polymerase simultaneously creates a new cDNA strand while displacing the previously made copy. The double stranded DNA containing the template strand is no longer needed.

The single stranded copy loops at the 3′ end as it binds to itself. FIP primer binds to the 5′ end of this single strand and accompanied by DNA polymerase, synthesizes a complementary strand. F3 primer, with DNA polymerase, binds to this end and generates a new double stranded DNA molecule while displacing the previously made single strand.

RT-LAMP amplification can be carried out in a mixture including a suitable buffer (such as a phosphate buffer or Tris buffer). The buffer may also include additional components, such as salts (such as KCl or NaCl, magnesium salts (e.g., MgCl₂ or MgSO₄), ammonium (e.g., (NH₄)₂SO₄)), detergents (e.g., TRITON-X100, TWEEN-20), or other additives (such as betaine or dimethylsulfoxide). The buffer or reaction mixture also includes nucleotides or nucleotide analogs. In some forms, an equimolar mixture of dATP, dCTP, dGTP, and dTTP (herein, dNTPs) is included, for example about 0.5-5 mM dNTPs (such as about 1-3 mM dNTPs).

A preferred RT-LAMP assay is exemplified herein as follows. All the reagents for LAMP reactions are assembled on ice and combined in a single reaction mixture of about 2.5 μL 10×isothermal amplification buffer (1×, 20 mM Tris-HCl (pH 8.8), about 10 mM (NH₄)₂SO₄, about 50 mM KCl, about 2 mM MgSO₄, 0.1% Tween 20), about 2.5 μL 10×Primer Mix (about 2 μM F3 about 2 μM B3; about 16 μM FIP about 16 μM BIP about 8 μM LF 8 μM LB), about 1.13 μL of 100 mM MgSO4, about 1.4 μL of 10 mM dNTP mix, about 2 μL of viral (clinical sample RNA), about 1 μL of 50 ng/ul Bst DNA polymerase (50 ng), about 1 ul of RTx reverse transciptase about 4 ng/ul and nuclease-free water to 25 μL. The mixture is incubated at 62° C. for 30 minutes. It is within the ability of one of ordinary skill in the art to vary the amount of reagent to effective amounts and conditions that result in amplification of the target RNA.

B. Cas12 Detection Assay

In some embodiments, the disclosed method takes advantage of the CRISPR-Cas 12 system. Type V CRISPR/Cas Cas12 proteins such as Cpf1 (Cas12a) and C2c1 (Cas12b) can promiscuously cleave non-targeted single stranded DNA (ssDNA) once activated by detection of a target DNA (double or single stranded). Once a type V CRISPR/Cas effector protein (e.g., a Cas12 protein such as Cas12a, Cas12b, Cas12c, Cas12d, Cas12e) is activated by a guide RNA, which occurs when the guide RNA hybridizes to a target sequence of a target DNA (i.e., the sample includes the targeted DNA), the protein becomes a nuclease that promiscuously cleaves ssDNAs (i.e., the nuclease cleaves non-target ssDNAs, i.e., ssDNAs to which the guide sequence of the guide RNA does not hybridize). Thus, when the target DNA is present in the sample (e.g., in some cases above a threshold amount), the result is cleavage of ssDNAs in the sample, which can be detected using any convenient detection method (e.g., using a labeled single stranded DNA oligonucleotide).

Cas12a is a single-RNA-guided nuclease, which only requires crRNA and contains a single RuvC domain. Upon target DNA binding, Cas12a cleaves both the target DNA in cis and non-target single-stranded NAs (ssDNAs) in trans. Guide RNA-target strand DNA hybridization conformationally activates Cas12a, triggering its trans-acting, non-specific, single-stranded DNase activity. The disclosed methods rely upon this non-specific single-stranded DNase activity, by introducing ssDNA activatable probes, cleavage of which activates the probe.

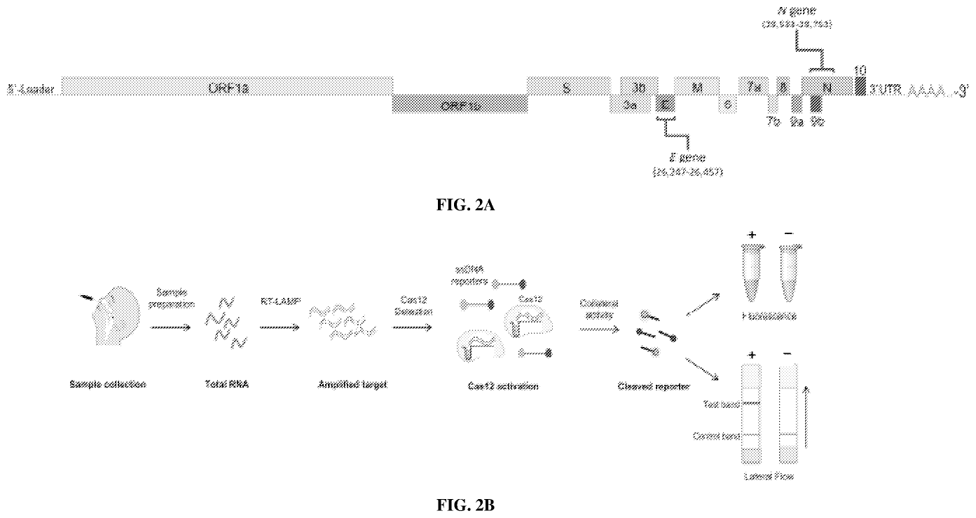

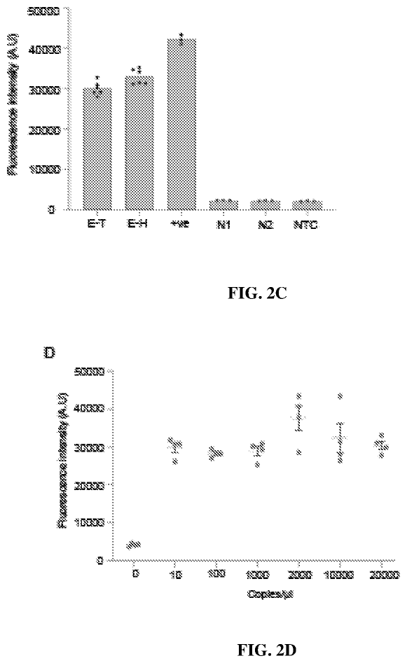

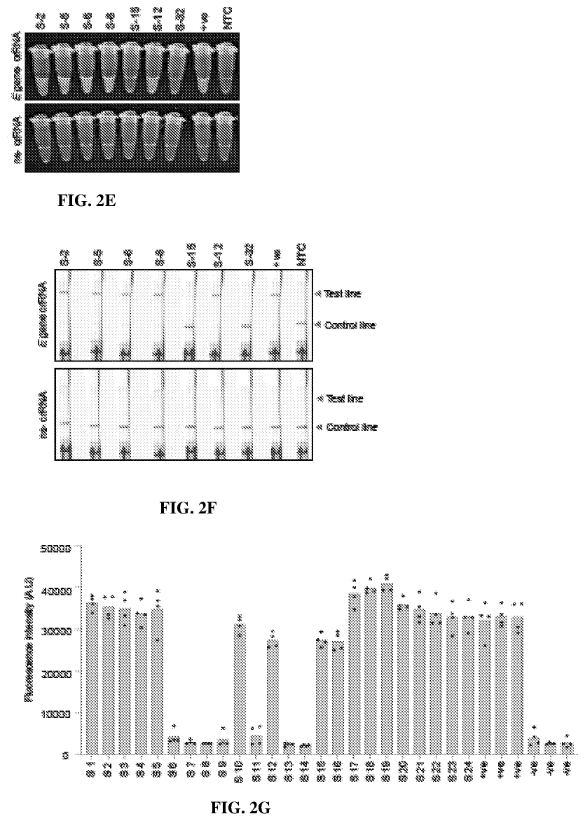

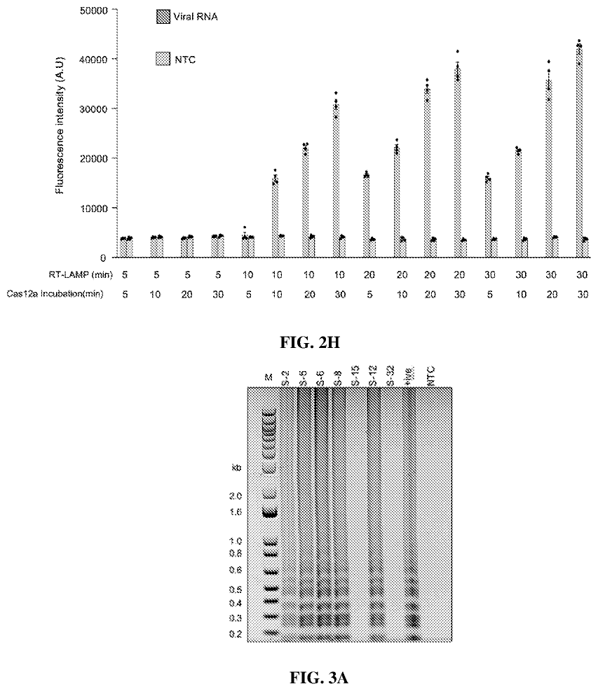

The RT-LAMP product is introduced to a Cas12 protein, for example, Cas12a or Cas12b protein pre-assembled with crRNAs specific to the E gene amplicon, non-specific crRNA, or without crRNA in the presence of FQ-ssDNA reporter. The Cas12 protein cleaves the ssDNA reporters only in the presence of specific crRNA and RT-LAMP amplicons, confirming the specificity and activity of this system (FIG. 2C).

The disclosed two-pot assays preferably use Cas12a. In this embodiment, the RT-LAMP amplification of SARS-CoV-2 nucleic acid is performed in the first tube. Then the RT-LAMP product is transferred to a second tube for Cas12a-based detection. Other embodiments use the one pot assay, and preferably employ Cas12b. Cas12b, a dual-RNA-guided nuclease containing a single RuvC domain and requiring both crRNA and tracrRNAs. Exemplary Cas12b effector proteins include Cas12b nuclease from Alicyclobacillus acidoterrestris (AacCas12b) and the AapCas12b, from the bacterium Alicyclobacillus acidophilus. For the one pot assay, RT-LAMP and Cas12b-based detection reagents, except for Cas12b-sgRNA complex, are mixed in a single reaction. Cas12b-sgRNA complex is temporarily separated from the reaction for example, by adding it on the tube wall. After sufficient time, for example, 30 min of RT-LAMP reaction, the Cas12b-sgRNA complex added onto the RT-LAMP reaction, for example, the Cas12b-sgRNA is centrifuged into the reaction mix for target cleavage and CRISPR/Cas-based detection, which takes place for an additional 15 min for example. In another embodiment, simultaneous RT-LAMP amplification and CRISPR-based detection of SARS-CoV-2 detection is performed in a single tube.

The method includes a) contacting a sample containing the RT-LAMP reaction product with: (i) a ribonucleoprotein including a Cas12 protein and a crRNA sequence that hybridizes with the target DNA (herein, the RT-LAMP reaction product); and (ii) a single stranded activatable detector DNA oligonucleotide that does not hybridize with the crRNA; and (b) measuring a detectable signal produced by cleavage of the single stranded detector DNA by the Cas12 protein, thereby detecting the RT-LAMP reaction product.

The Cas12 detection is exemplified herein as follows: 250 nM LbCas12a (500 nM for AaCas12b and AapCas12b) is first preincubated with 250 nM (500 nM for AaCas12b and AapCas12b) of specific (or non-specific) LbCas21a crRNAs in 1×Cas12 reaction Buffer for 30 min at 37° C. (62° C. for Cas12b) to assemble Cas12-crRNA ribonucleoprotein (RNP) complexes. The reaction is diluted 4 times with 1×binding buffer.

C. Cas13 Detection Assay

Cas13 protein complexes with the guide RNA via recognition of a short hairpin in the crRNA, and target specificity is encoded by the spacer that is complementary to the target region. In addition to programmable RNase activity, all Cas13s exhibit “collateral activity” after recognition and cleavage of a target transcript, leading to non-specific degradation of any nearby transcripts regardless of complementarity to the spacer. This collateral activity can be referred to as trans activity because the Cas13 is activated to cleave other RNA species in trans without sequence specificity. Conversely, the activity toward the targeted RNA transcript (e.g., having some complementarity to the spacer sequence of the crRNA) can be referred to as cis activity. The disclosed Cas13 based detection assay take advantage of this cis and trans Cas13 activity.

In some embodiments, the disclosed method takes advantage of the CRISPR-Cas13 system. Type VI CRISPR/Cas Cas13 proteins such as mCas13, TccCas13a, and HheCas13a can cleave non-targeted single stranded RNA (ssRNA) once activated by detection of a target RNA. Once a type VI CRISPR/Cas effector protein (e.g., mCas13, TccCas13a, or HheCas13a) is activated by a crRNA, which occurs when the crRNA hybridizes to a target sequence of a target RNA (i.e., the sample includes the targeted RNA), the protein becomes a nuclease that cleaves non-target ssRNAs (i.e., ssRNAs to which the spacer sequence of the crRNA does not hybridize). Thus, when the target DNA or RNA transcribed therefrom is present in the sample (e.g., in some cases above a threshold amount), the result is cleavage of ssRNAs in the sample, which can be detected using any convenient detection method (e.g., using a labeled ssRNA oligonucleotide).

Typically, the RT-LAMP product is transcribed, and the resulting RNA transcript is introduced to a Cas13 protein (e.g., mCas13, TccCas13a, or HheCas13a), which is pre-assembled with crRNAs specific to the N or E gene amplicon of SARS-CoV-2, non-specific crRNA, or without crRNA in the presence of FQ-ssRNA reporter. The Cas13 protein cleaves the ssRNA reporter only in the presence of specific crRNA and RNA transcript derived from, e.g., the N or E gene RT-LAMP amplicons, confirming the specificity and activity of the system.

In a specific embodiment, a Cas13-based method of detecting the presence of SARS-CoV-2 nucleic acid in a sample involves (a) contacting the sample with a set of RT-LAMP primers specific for the N or E gene of SARS-CoV-2 in an RT-LAMP reaction under conditions sufficient for amplification of the N or E gene of SARS-CoV-2 to generate an amplification product; (b) transcribing the amplification product to generate an RNA transcript; (c) contacting the RNA transcript with (i) a Cas13-based RNP complex composed of a Cas13 enzyme and a crRNA complementary to the RNA transcript; and (ii) an activatable ssRNA oligonucleotide; and (d) detecting cleavage of the ssRNA oligonucleotide by the Cas13 enzyme. In some embodiments, transcription of the amplification product in step (b) is mediated by a T7 RNA polymerase in vitro. Preferably, cleavage of the ssRNA oligonucleotide is dependent on or subsequent to binding of the Cas13-based RNP to the RNA transcript. In some embodiments, cleavage of the ssRNA oligonucleotide results in release of a previously quenched fluorescent signal, thereby indicating the presence of the SARS-CoV-2 nucleic acid.

The Cas13-based detection assay can be performed as a one-pot or two-pot assay depending on the Cas13 enzyme that is used.

It has been discovered that HheCas13a is a thermophilic/thermostable protein that can be used at elevated temperatures. The working Examples show that HheCas13a is a thermophilic/thermostable protein that exhibits efficient in cis and in trans cleavage activities at high temperatures. For example, HheCas13a exhibited robust activity at temperatures up to 60° C. Therefore, it can be used for applications requiring elevated temperatures.

The working examples demonstrate that previously uncharacterized protein TccCas13a maintained a robust activity at temperatures as high as 70° C. and thus, it can be used for applications requiring elevated temperatures.

Thus, in some embodiments when TccCas13a or HheCas13a is used in the Cas13-based detection assay, the assay can be performed as a one-pot assay in which reverse transcription and LAMP isothermal amplification of the target nucleic acids, coupled with T7-mediated in vitro transcription and Cas13-based detection of the amplified and in vitro-transcribed target RNA is carried out in the same tube. In an exemplary embodiment, the one-pot reaction can be performed using RT-LAMP primers with final concentrations of 1.6μM FIP/BIP primers (with the T7 promoter sequence fused to either the FIP or BIP primer), 0.2 μM F3/B3 primers, and 0.4 μM LF/LB primers, 1×Isothermal Amplification Buffer, 1.4 mM dNTPs, Bst DNA Polymerase, 0.45 U/uL of WarmStart RTx Reverse Transcriptase or 2 U/uL SuperScript IV reverse transcriptase, 6 mM MgSO4, 0.1 U/uL thermostable RNAseH, 0.8 U/uL RNaseOUT or RNasin plus, 0.5 mM NTPS, 4 U/μL Hi-T7 RNA polymerase, 0.4 U/μL thermostable inorganic pyrophosphatase, 250 nM RNA reporter, 50 nM Cas13, 50 nM crRNA, and 2 μL of template RNA in a 25-4, reaction. The reaction can be incubated in a 96 well-plate (BioRad) at 56° C. for 1-2 hours.

SHERLOCK (Gootenberg, et al., Science, 2017) is a Cas13-based nucleic acid detection that uses LwaCas13a enzyme in two-pot assays. Attempts to use SHERLOCK in one-pot with the RT-RPA amplification method at 37 C, exhibited low sensitivity thus subsequent SHERLOCK work was based on two-pot assays (e.g., Barnes et at, Nature communication, 2020). Although SHERLOCK was modified to be coupled with RT-LAMP (SHERLOCK CRISPR SARS-CoV-2 Kit_IDT), this is still a two-pot assay as LwaCas13a cannot be used in one-pot at elevated temperatures with RT-LAMP. This complicates the use of this approach for real diagnostic settings.

The disclosed one-pot TccCas13a/HheCas13a based detection assay is advantageous over the two-pot SHERLOCK and provides substantial sensitivity and specificity, a simpler one-pot reaction that significantly helps in avoiding cross contamination (a common problem with two-pot assays), and simple and easy read-out that allows point-of-care use, and relatively short running time.

mCas13 is a previously uncharacterized Cas13 enzyme. This disclosure is believed to be the first characterization of the in cis and in trans activities of mCas13 and utilization of mCas13 protein for diagnostic purposes. The working Examples demonstrate that mCas13 is active at temperatures of about 37° C. to about 42° C. with the highest activity observed at 37° C. Thus, because of its incompatibility with the elevated temperatures required for the RT-LAMP reaction, mCas13 is preferably used in a two-pot assay.

In the two-pot assay, the RT-LAMP reaction is performed in a first tube while T7-mediated in vitro transcription and mCas13-based detection of the amplified and in vitro-transcribed target RNA is performed in a second tube.

In some embodiments of the two-pot Cas13 based assay, the first step involves performing reverse transcription and isothermal amplification of target nucleic acids (RT-LAMP) at about 62° C. for example, for about 35 minutes. The second step involves T7-mediated in vitro transcription and mCas13-based detection of the amplified and in vitro-transcribed target RNA in another tube. In an exemplary embodiment, 2 μL of the RT-LAMP reaction product can be combined with 1×cleavage buffer, 500 nM mCas13/crRNA assembled RNPs, 25 units T7 RNA polymerase, 1 mM NTPs, and 250 nM RNA reporter in a 20-μL reaction. The reaction can be incubated at 37° C. for 20-30 minutes.

D. Measuring a Detectable Signal Using Activatable Labeled Oligonucleotides

For fluorescence-based detection, an exemplary reaction provided herein is as follows: for Cas12a 250 nM and for Cas12b, 500 nM of fluorophore-quencher (FQ) reporter and 2 ul of the RT-LAMP reaction was added to the pre-assembled Cas12-sgRNA RNP complexes and incubated at 37° C. (62° C. for Cas12b) for 10-30 mins. End-point fluorescence detection can be monitored using a Tecan plate reader.

The detection of a target nucleic acid can include a step of measuring a detectable signal (e.g., measuring a detectable signal produced by Type V or VI CRISPR/Cas effector protein (e.g., a Cas12 protein such as Cas12a, Cas12b, Cas12c, Cas12d, Cas12e or a Cas13 protein such as mCas13, TccCas13a, HheCas13a) generated by ssDNA/RNA cleavage. Because a Type V CRISPR/Cas effector protein (e.g., a Cas12 protein such as Cas12a, Cas12b, Cas12c, Cas12d, Cas12e) cleaves non-targeted ssDNA once activated, which occurs when a guide RNA hybridizes with a target DNA in the presence of a Type V CRISPR/Cas effector protein (e.g., a Cas12 protein such as Cas12a, Cas12b, Cas12c, Cas12d, Cas12e), a detectable signal can be any signal that is produced when ssDNA is cleaved. Likewise, a detectable signal can be any signal that is produced when ssRNA is cleaved by a Type VI CRISPR/Cas effector protein (e.g., a Cas13 protein such as mCas13, TccCas13a, HheCas13a).

Cleavage of a labeled detector ssDNA/ssRNA can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA/ssRNA can be detected by a color-shift. Such a shift can be expressed as a loss of an amount of signal of one color (wavelength), a gain in the amount of another color, a change in the ration of one color to another, etc.

In some embodiments, fluorescence can be detected by a portable or hand-held fluorescence visualizer such as the P51 Molecular Fluorescence Viewer. Such devices are adapted to easily visualize and interpret the results by illuminating a reaction tube with blue light. Fluorescent reactions become visible through a film used as an optical filter, thus allowing fluorescence to be readily visible to the human eye and obviating the need for plate readers, etc.

In some embodiments, cleavage of the activatable labeled ssDNA/ssRNA oligonucleotide is detected through lateral flow detection. In an exemplary embodiment, the labeled ssDNA/ssRNA oligonucleotide can be added to the Cas12 or Cas13 reaction along with the RT-LAMP product or RNA transcript. Upon incubation for a suitable length of time, lateral flow readout can be performed by placing lateral flow dipsticks (e.g., Milenia HybriDetect 1, TwistDx, cat. no. MILENIA01) into the Cas12/Cas13 detection reaction. In some embodiments, the lateral flow results may be interpreted as follows, a single band, close to the sample pad indicates a negative result (control band), whereas a single band close to the top of the strip (test band) or two bands (test and control) indicate a positive result. The use of lateral flow immunoassay for the detection of nucleic acids is known in the art and one of skill in the art is readily able to adapt it for use herein using commercially available lateral flow cleavage reporter oligonucleotides and dipsticks. The ssDNA or ssRNA reporter for the lateral flow assay can include FAM and/or biotin moieties. Exemplary suitable reporters include 5′-/56-FAM/TTTTTTT/3Bio/-3′ and variants thereof in which the oligonucleotide sequence between the FAM and Biotin (Bio) moieties is replaced with oligonucleotide sequences disclosed herein (e.g., from the “Activatable oligonucleotide reporter” section or Table 7), or otherwise known in the art. In some embodiments, Milenia HybriDetect 1, TwistDx, cat. no. MILENIA01, lateral flow dipsticks are used.

In some embodiments, the step of measuring can include one or more of: gold nanoparticle based detection (e.g., see Xu et al., Angew Chem Int Ed Engl. 2007; 46(19):3468-70; and Xia et al., Proc Natl Acad Sci USA. 2010 107(24):10837-41), fluorescence polarization, colloid phase transition/dispersion (e.g., Baksh et al., Nature. 2004 Jan. 8; 427(6970):139-41), electrochemical detection, semiconductor-based sensing (e.g., Rothberg et al., Nature. 2011 Jul. 20; 475(7356):348-52; e.g., one could use a phosphatase to generate a pH change after ssDNA/ssRNA cleavage reactions, by opening 2′-3′ cyclic phosphates, and by releasing inorganic phosphate into solution), and detection of a labeled detector ssDNA (see elsewhere herein for more details). The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

### IV. Kits

Also disclosed are kits for carrying out the disclosed methods. Compositions, reagents, and other materials can be packaged together in any suitable combination as a kit useful for performing, or aiding in the performance of, the disclosed methods. It is useful if the kit components in a given kit are designed and adapted for use together in the disclosed methods. For example, disclosed are kits with one or more primers, buffers, and/or enzymes. The kits may include a sterile needle, swab, syringe, ampule, tube, container, or other suitable vessels for isolating samples, holding assay components and/or performing the assay. The kits may include instructions for use.

The kit can include a sufficient quantity of reverse transcriptase, a DNA polymerase, RNA polymerase, CRISPR/Cas effector protein, crRNA, primers (e.g., 2 or more primer pairs), activatable ssDNA/ssRNA oligonucleotides, buffer(s), or any combination thereof, for the detection assay described herein. A kit may further include instructions pertinent for the particular embodiment of the kit, such as providing conditions and steps for operation of the method. A kit may also contain reaction containers such as microcentrifuge tubes and the like. A kit may also reagents for isolating a biological sample and extracting nucleic acids therefrom.

The kits may contain nucleic acid primers suspended in an aqueous solution or as a freeze-dried or lyophilized powder, for instance. The container(s) in which the primers are supplied can be any conventional container that is capable of holding the supplied form, for instance, microfuge tubes, multi-well plates, ampoules, or bottles. One or more control probes, primers, and or nucleic acids also may be supplied in the kit. For example, the kit may include one or more positive control samples (such as a sample including a particular viral nucleic acid) and/or one or more negative control samples (such as a sample known to be negative for a particular viral nucleic acid).

In some embodiments, one or more primers (such as one or more sets of primers suitable for SARS-Cov-2), may be provided in pre-measured single use amounts in individual, typically disposable, tubes, wells, or equivalent containers. In this embodiment, the sample to be tested for the presence of the target nucleic acids can be added to the individual tube(s) or well(s) and amplification and/or detection can be carried out directly. In some examples, the containers may also contain additional reagents for amplification reactions, such as buffer, enzymes (such as reverse transcriptase, DNA polymerase, RNA polymerase, and/or CRISPR/Cas effector protein), dNTPs, or other reagents. In some embodiments, the container includes all of the components required for the reaction except the sample (and water, if the reagents are supplied in dried or lyophilized form).

In some embodiments, the kit can contain reagents and instructions for detecting a viral nucleic acid. This can include for example, reagents, instructions, software and/or hardware for performing the method and analysis of results.

The present invention will be further understood by reference to the following non-limiting examples.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Example 1: iSCAN Permits Rapid, Specific, and Sensitive Detection of SARS-CoV-2

Materials and Methods

Nucleic Acid Preparations

The control SARS-CoV-2 viral RNA sequences used in this study were synthetic RNA from Twist Bioscience, 102024 diluted to (1×10³ RNA copies/microliter).

For Cas12 DNA cleavage assays, dsDNA substrates were generated by either PCR amplification from 2019-nCoV_N_Positive Control vector (IDT, 10006625) using forward (N gene-F) and reverse primer (N gene-R) (Table 1) for N gene.

TABLE 1
Primers used for in vitro transcription and PCR amplification
of target genes.

| Primer Name | Sequence 5′-3′ | Purpose |
|---|---|---|
| N gene-F | GAAATTAATACGACTCACTATAG | To PCR amplify N gene-2 out |
| | GGAGGCTTCTAAGAAGCCTCGG | of IDT kit control. |
| | C (SEQ ID NO: 1) | |
| N gene-R | GGCTCTGTTGGTGGGAATG (SEQ | To PCR amplify N gene-2 out |
| | ID NO: 2) | of IDT kit control. |
| T7-3G IVT | GAAATTAATACGACTCACTATAG | To anneal with Cas12a crRNA |
| primer | GG (SEQ ID NOG) | oligos for in vitro transcription |
| | | To use as F primer to amplify |
| | | Cas 12b sgRNAs |

Alternatively, dsDNA was designed and ordered as dsDNA gBlocks (IDT) for E gene (Table 2).

TABLE 2
SARS-CoV-2 gBlocks targeted sequences

| | |
|---|---|
| E | gaaattaatacgactcactataggGGGAACCAATTTATGATGAAC |
| gene | CGACGACGACTACTAGCGTGCCTTTGTAAGCACAAGCTGATGAGT |
| | ACGAACTTATGTACTCATTCGTTTCGGAAGAGACAGGTACGTTAA |
| | TAGTTAATAGCGTACTTCTTTTTCTTGCTTTCGTGGTATTCTTGC |
| | TAGTTACACTAGCCATCCTTACTGCGCTTCGATTGTGTGCGTACT |
| | GCTGCAATATTGTTAACGTGAGTCTTGTAAAACCTTCTTTTTACG |
| | TTTACTCTCGTGTTAAAAATCTGAA (SEQ ID NO: 4) |

The gblocks were cloned into pJet2.1 (Thermo Scientific, K1231) vector and the resulting plasmids. PCR products were gel purified using the QIAquick Gel Extraction kit following manufacturer's instructions.

To assess the activity of the purified Bst LF via LAMP reaction, the N gene PCR amplicon was used as template in the LAMP reaction. In vitro transcription was carried out using TranscriptAid T7 High Yield Transcription Kit (Thermo Scientific K0441) following manufacturer's instructions. For LbCas12a crRNAs, templates for in vitro transcription were generated using single-stranded DNA oligos containing a T7 promoter, scaffold and spacer in reverse complement orientation (IDT), which were then annealed to T7 forward primer in taq DNA polymerase buffer (Invetrogen). For Cas12b crRNAs, the AacCas12b 90 nt-long crRNA scaffold was synthesized and ordered as sense and anti-sense ssDNA ultramers containing the T7 promoter at the 5′ end (IDT). The two ssDNA strands were annealed in taq DNA polymerase buffer to generate T7-Cas12b crRNA scaffold. The scaffold was used as a template for PCR with T7 forward primer and reverse primers containing 20 nt spacer sequences. All DNA oligos and substrates are listed in (Table 1 and 3).

TABLE 3
LbCas12a crRNAs and Cas12b sgRNAs

| Cas | | Genomic |
|---|---|---|
| Enzyme | gRNA Sequence 5′-3′ | Region |
| LbCas12a | GTAACTAGCAAGAATACCACATCTACACTTAGTAGAAATTACC | E gene |
| | CTATAGTGAGTCGTATTAATTTC (SEQ ID NO: 5) | |
| | GGACTTCCCTATGGTGCTAAATCTACACTTAGTAGAAATTACC | N gene |
| | CTATAGTGAGTCGTATTAATTTC (SEQ ID NO: 6) | |
| Cas12b | ATATTGCAGCAGTACGCACAGTGCCACTTCTCAGATTTGAG | E gene |
| | (SEQ ID NO: 7) | |
| | GCTGGACTTCCCTATGGTGCGTGCCACTTCTCAGATTTGAG | N gene |
| | (SEQ ID NO: 8) | |
| Cas12b | GAAATTAATACGACTCACTATAGGGTCTAGAGGACAGAATTTT | |
| scaffold-F | TCAACGGGTGTGCCAATGGCCACTTTCCAGGTGGCAAAGCCCG | |
| | TTGAGCTTCtcaaatctgaGAAGTGGCAC (SEQ ID NO: 9) | |
| Cas12b | GTGCCACTTCtcagatttgaGAAGCTCAACGGGCTTTGCCACCTGGA | |
| scaffold-R | AAGTGGCCATTGGCACACCCGTTGAAAAATTCTGTCCTCTAGA | |
| | CCCTATAGTGAGTCGTATTAATTTC (SEQ ID NO: 10) | |

The in vitro transcribed sgRNAs were purified using MEGAclear™ Transcription Clean-Up Kit (Thermo Scientific AM1908) following manufacturer's instructions.

Protein Purification

Bst DNA polymerase, large fragment was induced at OD₆₀₀ 0.7 with 100 ng/mL tetracycline-HCl at 37° C. for 3 hours and the pellet was collected. Bst DNA polymerase was purified following Li et. al 2017 protocol [35, 42]. The collected protein fractions were quantified, and aliquots were flash frozen in liquid nitrogen.

RTx reverse transcriptase was purified following Bhadra et. al 2020 protocol [43]. The collected protein fractions were quantified, and aliquots were flash freezed in liquid nitrogen. LbCas12a, AaCas12b, AapCas12b recombinant proteins were purified following Chen et. al 2018 protocol [30]

Assessment of the Activity and Functionality of In-House Produced Enzymes.

To confirm the polymerization activity of the purified Bst DNA polymerase enzyme LAMP reaction was assembled and carried out at 62° C. The commercial Bst enzyme Bst 2.0 WarmStart DNA Polymerase (NEB) was used as per manufacturer's instructions as control. As initial confirmation, the reported LAMP primers designed against SARS-CoV-2 were used [37] (Supplementary table1).

The activity of the purified RTx reverse transcriptase enzyme was confirmed by single enzyme RT-PCR reaction following Bhadra et. al 2020 protocol [43]. The Synthetic SARS-CoV-2 RNA control (102024 Twist Bioscience) was used as input RNA template. The one step RT-PCR was carried out with cycling conditions: 60° C. for 30 minutes, 95° C. for 10 minutes and 45 cycles of 95° C. for 15 seconds followed by 55° C. for 30 seconds and 68° C. The amplified product was separated on 1% agarose gel stained with SYBR-safe dye.

The dsDNA cleavage and collateral ssDNA degradation activities of Cas12a and Cas12b proteins were confirmed following previously described protocols [30, 38]

Design and Screening of LAMP Primers

Different primer sets targeting several regions of the N gene and E gene of the SARS-CoV-2 genome (GenBank accession number MN908947) were designed using PrimerExplorer v5 software (https://primerexplorer.jp/e/). Optimal sets that showed the best performance were determined by conducting LAMP assays to detect specific targets. (Table 1).

TABLE 4
LAMP Primers

| Primer Name | Sequence 5′-3′ | Targeted region |
|---|---|---|
| Gene N-B-F3 * | ACCGAAGAGCTACCAGACG (SEQ ID NO: 11) | N gene-1 |
| Gene N-B-B3 * | TGCAGCATTGTTAGCAGGAT (SEQ ID NO: 12) | N gene-1 |
| Gene N-B-FIP * | TCTGGCCCAGTTCCTAGGTAGTTCGTGGTGGTG | N gene-1 |
| | ACGGTAA (SEQ ID NO: 13) | |
| Gene N-B-BIP* | AGACGGCATCATATGGGTTGCACGGGTGCCAA | N gene-1 |
| | TGTGATCT (SEQ ID NO: 14) | |
| Gene N-B-LF * | CCATCTTGGACTGAGATCTTTCATT (SEQ ID | N gene-1 |
| | NO: 15) | |
| Gene N-B-LB * | ACTGAGGGAGCCTTGAATACA (SEQ ID NO: 16) | N gene-1 |
| F3-N3-1 | CCGAAGAGCTACCAGACGAA (SEQ ID NO: 17) | N gene-2 |
| B3-N3-1 | TGTAGCACGATTGCAGCATT (SEQ ID NO: 18) | N gene-2 |
| FIP-N3-1 | TCTGGCCCAGTTCCTAGGTAGTGGTGGTGACG | N gene-2 |
| | GTAAAATGAAAG (SEQ ID NO: 19) | |
| BIP-N3-1 | AGACGGCATCATATGGGTTGCACGGGTGCCAA | N gene-2 |
| | TGTGATCT (SEQ ID NO: 20) | |
| LF-N3-1 | AGAAATACCATCTTGGACTGAG (SEQ ID NO: 21) | N gene-2 |
| LB-N3-1 | ACTGAGGGAGCCTTGAATACAC (SEQ ID NO: 22) | N gene-2 |
| F3-E-2 | GTACTCATTCGTTTCGGAAG (SEQ ID NO: 23) | E gene |
| B3-E-1 | AGGAACTCTAGAAGAATTCAGAT (SEQ ID | E gene |
| | NO: 24) | |
| FIP-E-2 | GGATGGCTAGTGTAACTAGCAAGGGTACGTTA | E gene |
| | ATAGTTAATAGCGT (SEQ ID NO: 25) | |
| BIP-E-2 | CGCTTCGATTGTGTGCGTACGAGAGTAAACGT | E gene |
| | AAAAAGAAGGTT (SEQ ID NO: 26) | |
| LF-E-2 | ACCACGAAAGCAAGAAAAAG (SEQ ID NO: 27) | E gene |
| LB-E-1 | GCTGCAATATTGTTAACGTGAGTCT (SEQ ID | E gene |
| | NO: 28) | |

* Primers used in [1].

Screening for the best performing and most sensitive LAMP primers was done by running RT-LAMP as described below.

Two-Pot iSCAN Detection Assay

The iSCAN detection assays were performed in two steps. In the first step, RT-LAMP was performed to reverse transcribe and pre-amplify the viral RNA to generate dsDNA substrates for Cas12 enzymes. All the reagents for LAMP reactions were assembled on ice and combined in a single reaction mixture of 2.5 μL 10×isothermal amplification buffer (1×, 20 mM Tris-HCl (pH 8.8), 10 mM (NH₄)₂SO₄, 50 mM KCl, 2 mM MgSO₄, 0.1% Tween 20), 2.5 μL 10×Primer Mix (2 μM F3 2 μM B3 16 μM FIP 16 μM BIP 8 μM LF 8 μM LB), 1.13 μL of 100 mM MgSO4, 1.4 μL of 10 mM dNTP mix, 2 μL of viral (control or clinical sample RNA), 1 μL of 50 ng/ul Bst DNA polymerase (50 ng), 1 ul of RTx reverse transciptase 4 ng/ul and nuclease-free water to 25 μL. Mixtures were incubated at 62° C. for 30 minutes or as indicated.

Next, for Cas12 detection assays, 250 nM LbCas12a (500 nM for AaCas12b and AapCas12b) was first preincubated with 250 nM (500 nM for AaCas12b and AapCas12b) of specific (or non-specific) LbCas21a crRNAs in 1×Cas12 reaction Buffer for 30 min at 37° C. (62° C. for Cas12b) to assemble Cas12-crRNA ribonucleoprotein (RNP) complexes. The reaction was diluted 4 times with 1×binding buffer. For fluorescence-based detection, for Cas12a 250 nM and for Cas12b, 500 nM of fluorophore-quencher (FQ) reporter (5′-/56-FAM/TTATT/3IABKFQ/-3′, IDT) or DNase alert substrate (11-02-01-02, IDT) and 2 ul of the RT-LAMP reaction was added to the pre-assembled Cas12-sgRNA RNP complexes and incubated at 37° C. (62° C. for Cas12b) for 10-30 mins. End-point fluorescence detection was monitored using a Tecan plate reader (Tecan 200).

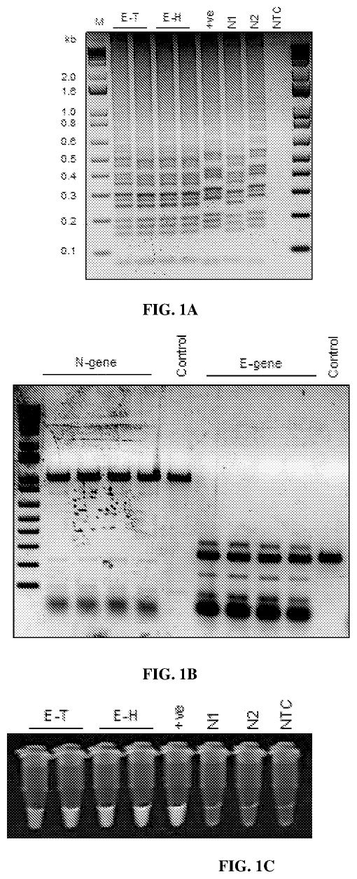

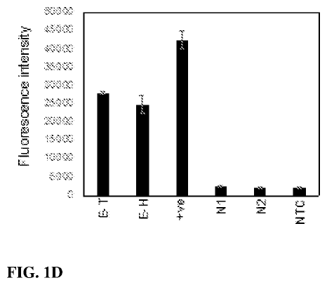

For lateral flow detection, the Cas12 detection reaction was assembled as described above with modifications. Lateral flow cleavage reporter (5′-/56-FAM/TTATTATT/3Bio/-3′, IDT) was added to the reaction at a final concentration of 500 nM in 100 ul reaction volume along with the 2 ul RT-LAMP product, and the reaction was incubated at 37 C° (62° C. for Cas12b) for 10-30 minutes. Upon completion of the reaction, lateral flow readouts were performed by placing lateral flow dipsticks (Milenia HybriDetect 1, TwistDx, cat. no. MILENIA01) into Cas12 detection reaction and results were observed after 5 minutes. Interpretation of lateral flow results was done as follows, a single band, close to the sample pad indicated a negative result (control band), whereas a single band close to the top of the strip (test band) or two bands (test and control) indicated a positive result (FIG. 1B).

One-Pot iSCAN Detection Assays

To develop one-pot iSCAN detection system with AapCas12b, different strategies were employed; 5 μL of 5 μM AapCas12b was spotted to the walls of the tube [44, 45] or directly added to the initial 50 μL reaction mix, independently. The initial 50 μL of reaction mix contained the complete RT-LAMP reagents such as 5 μL 10×isothermal amplification buffer, 5 μL 10×Primer Mix, 4 μL of 100 mM MgSO4, 7 μL of 10 mM dNTP mix, 2 μL of Bst DNA polymerase (50 ng), and 2 ul of RTx reverse transcriptase 4 ng/ul, together with 2.5 μL of 500 ng/μL salmon sperm DNA, 2 μL of viral (control or clinical sample) RNA, 5 μL of 5 μM specific (or non-specific) AacCas21b crRNAs and 5 μL of 5 μM lateral flow cleavage reporter (5′-/56-FAM/TTATTATT/3Bio/-3′, IDT) and 5.5 μL nuclease-free water. After the addition of all reagents, the reaction tubes were incubated horizontally in a water bath at 62° C. for 30 minutes, then the tubes were spun down to mix the AapCas12b with the LAMP reaction, and incubated again at 62° C. for 30 minutes for further AapCas12b based collateral activity. In case of AapCas12b direct addition to the total reaction mix, the tubes were incubated in heat block at 62° C. for 1 hr. Each reaction was carried out in duplicates. Later, for the lateral flow-based detection the two 50 ul duplicate reaction mixes were combined into one tube (100 ul) and carried out lateral flow detection as described earlier.

Clinical Sample Collection and RNA Extraction

Oropharyngeal and nasopharyngeal swabs were collected by the physicians from COVID-19 suspected patients and placed in 2-mL screw-capped cryotubes containing 1 mL of TRIZOL to be inactivated and transported to King Abdullah University of Science and Technology. The sample tubes were sprayed with 70% ethanol, enveloped with absorbent tissue and placed in individual and sealed biohazard bags with labels accordingly. The bags were then placed in leak-proof boxes and sprayed with 70% ethanol before being placed in a dry ice container to be transferred.

Sample RNA of SARS-CoV-2 was then extracted following instructions as described in the CDC EUA-approved protocol using the DIRECTZOL KIT from ZymoBiomics (Direct-Zol RNA Miniprep (Zymo Research); catalog #R2070) following the manufacturer's instructions.

Real-Time Reverse Transcription PCR (RT-PCR) for Detection of Positive SARS-CoV2 RNA Sample.

RT-PCR was conducted by using the oligonucleotide primer/probe (Integrated DNA Technologies, catalog #10006606) and Superscript III one-step RT-PCR system with Platinum Taq Polymerase (catalog #12574-026) as per the manufacturer's protocol on extracted RNA sample.

The RNA sample for SARS nCoV 2019 considered as positive when the cycle threshold (Ct) value for both E and N genes was <36 and negative when the Ct value was more than >36 for both genes.

Results

Establishment of RT-LAMP for Rapid Detection of SARS-CoV-2 in a Low-Resource Environment

SARS-CoV-2 testing faces many hurdles, including the availability of critical reagents, highly-trained technical personnel, and sophisticated equipment. Hence, the ability to produce diagnostic reagents in-house is vital in epidemic situations. Therefore, studies are conducted to build a SARS-CoV-2 end-to-end detection platform that is sensitive, specific, easy to use, and low cost to facilitate its field deployment on a massive scale. LAMP reactions employ DNA polymerases that possess strand-displacement activities, such as the Bst DNA polymerase large fragment, for efficient amplification of the target DNA [21]. By supplementing the LAMP reaction with a suitable isothermal reverse transcriptase, reverse transcription LAMP (RT-LAMP) can be used to detect RNA by reverse transcribing the target RNA and subsequent DNA amplification in one step. Therefore, recombinant Bst DNA polymerase large fragment from Geobacillus stearothermophilus was expressed and purified [35], and the synthetic, directed evolution-derived RT ‘xenopolymerase’ RTx enzymes [36]. The catalytic activities and performance of the purified proteins for LAMP with the purified Bst LF, and RT-LAMP with RTx and Bst LF were assayed and compared with commercial Bst and RTx enzymes using published RT-LAMP primers designed against the SARS-CoV-2 genome [37]. The in-house produced proteins showed efficient and consistent LAMP and RT-LAMP amplifications comparable to the commercial enzymes (FIG. 1A).

Next, several sets of LAMP primers targeting the SARS-CoV-2 genome were designed, built and tested. The SARS-CoV-2 genome consists of ˜30 kb positive single-stranded RNA with a 5′-cap structure and 3′ poly-A tail containing several genes characteristic of coronaviruses, such as S (spike), E (envelope), M (membrane), and N (nucleocapsid) genes (FIG. 2A). Other elements of the genome, such as ORF1a and ORF1b, encode non-structural proteins, including RNA-dependent RNA polymerase (RdRp) [8, 9]. Two regions in the N and E genes were targeted. The N gene at the 3′ end of the virus genome is highly conserved among coronaviruses. LAMP primers were designed to generate ˜200 bp amplification products to ensure robust amplification sufficient for LAMP-based detection. RT-LAMP experiments were conducted on synthetic SARS-CoV-2 sequences. The N primer sets were able to specifically and efficiently amplify the synthetic virus fragments, but not the controls.

Efficient Detection of SARS-CoV-2 Via RT-LAMP Coupled with CRISPR-Cas12a

The sensitivity of RT-LAMP is comparable with the RT-PCR. However, the specificity of the reactions and the visualization of the results may be complicated due to primer-dimer formation, non-specific amplification, and cross-contamination when running a large number of samples simultaneously. Therefore, to develop a highly sensitive and specific detection system, CRISPR-Cas12 was coupled with RT-LAMP target amplification to establish a binary system for SARS-CoV-2 detection. To this end, the class II, type V Lachnospiraceae bacterium ND2006 (LbCas12a) orthologue was purified.

The programmable (specific) cis-cleavage activity of LbCas12a was assessed against dsDNA targets and (collateral) trans-cleavage activity on ssDNA non-targeted sequences. Purified LbCas12a assembled with crRNAs targeting dsDNA fragments of the SARS-CoV-2 N or E genes exhibited endonuclease activity on the targeted dsDNA, confirming the catalytic cis-cleavage activity of the purified Cas12a (FIG. 1B). To assess the collateral activity of the purified LbCas12a protein, the specific dsDNA targeting reaction was supplemented with non-targeted fluorophore quencher (FQ)-labeled ssDNA reporters. Cas12a catalyzed the efficient degradation of the ssDNA FQ reporters as measured by the fluorescent signal, which was generated only in the presence of the specific dsDNA targets and specific crRNA, indicating the active collateral cleavage activity of the purified enzyme (FIG. 1C, D).

To establish the disclosed iSCAN system for the detection of SARS-CoV-2, RT-LAMP for virus detection and amplification was coupled with CRISPR-Cas12 as a specificity factor. The positive signal from Cas12 provides a specific and accurate signal for virus detection. Collateral ssDNA-FQ reporter is cleaved upon Cas12a binding and cleavage of the target virus sequence in the RT-LAMP amplification products (FIG. 2B). CrRNAs were designed, specific to the SARS-CoV-2 N and E gene genomic regions amplified with the primer sets identified in the primer screening assays.

The activity and specificity of the two-pot iSCAN system was tested using an FQ reporter assay. RT-LAMP was performed using the E gene-specific RT-LAMP primers and synthetic SARS-CoV-2 genome. Subsequently, the RT-LAMP product was introduced to Cas12a protein pre-assembled with crRNAs specific to the E gene amplicon, non-specific crRNA, or without crRNA in the presence of FQ-ssDNA reporter. Cas12a cleaved the ssDNA reporters only in the presence of specific crRNA and RT-LAMP amplicons, confirming the specificity and activity of this systems (FIG. 2C).

Next, studies were conducted to quantitatively compare the sensitivity of this system to that of the approved SARS-CoV-2 CDC RT-qPCR assays to determine the limit of detection (LoD). The two-pot iSCAN assays were performed with various dilutions of the synthetic viral RNA ranging from 0 to 20000 copies per reaction. The LoD assays showed that the iSCAN reactions could detect down to 10 RNA copies per reaction, indicating the high sensitivity of our assay (FIG. 2D).

To further optimize the assay, different RT-LAMP and Cas12a detection timing were systemically evaluated to determine the time needed to detect the signal. Using the lowest concentration identified in the LoD assay, RT-LAMP products from different amplification time points were added, including 5, 10, 20, and 30 min to crRNA-Cas12a detection complex along with the fluorescence reporter. The samples were incubated at 37° C. for different times (5, 10, 20, and 30 min), and the fluorescent signal generated from the reporter by the Cas12a collateral activity upon target recognition was measured. The disclosed assay reliably detected the positive signal with 20 min of RT-LAMP followed by 20 min of Cas12a detection comparable to the stronger fluorescent signal with prolonged RT-LAMP and Cas12a detection (FIG. 2H).

Validation of RT-LAMP Coupled CRISPR-Cas12a for the Detection of SARS-CoV-2 in Clinical Samples

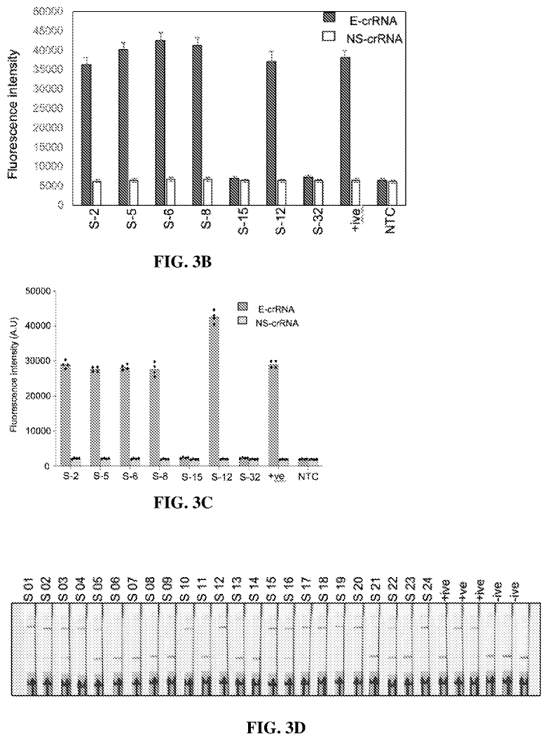

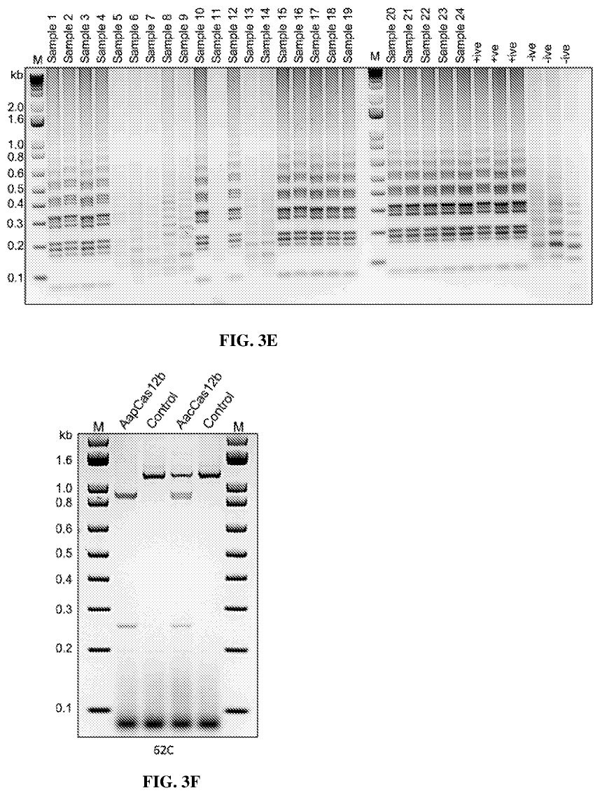

To validate the disclosed detection system for application with real samples from patients, the capability of the iSCAN assay was first assessed to detect SARS-CoV-2 nucleic acid extracted from nasopharyngeal swabs of five different patients who tested positive for SARS-CoV-2 with RT-qPCR assays and two patients who tested negative. The positive samples had different Ct values ranging from 15 to 40 with the CDC qPCR N gene primer set (IDT, 10006606). Using fluorescence-based detection with at least three replicates for each sample, the iSCAN system targeting the SARS-CoV-2 E gene showed 100% agreement with RT-qPCR results (FIG. 2E and FIG. 3A-3C).

To facilitate the effective detection of SARS-CoV-2 in a low-resource environment, studies were conducted to simplify the use of iSCAN for the detection of SARS-CoV-2. Therefore, lateral flow immunochromatography was coupled with the iSCAN detection system. Lateral flow strips are user-friendly and straightforward, and can facilitate the development of home testing for SARS-CoV-2. The disclosed system was evaluated for POC diagnostics with lateral flow readouts using commercial lateral flow strips. Lateral flow assays showed 100% concordance with results obtained with fluorescence-based detection (FIG. 2F).

Next, the iSCAN assay was evaluated by testing an additional 24 nasopharyngeal swab samples obtained from 21 SARS-CoV-2-positive and 3 negative patients. The performance of the disclosed system was assayed with E and N gene primers and crRNAs. A low sensitivity was observed when using the E gene target, with 8 out of 21 positive samples testing positive, and 3 out of 3 negative samples testing negative with fluorescence-based readouts data not shown). However, the N gene assay was used, a significant increase in the sensitivity of the assay was observed, where 18 samples tested positive out of the 21 positive samples with qPCR (˜86%), and the 3 negative samples were diagnosed correctly in agreement with the qPCR data (100%) (FIG. 2G). The performance the iSCAN N gene assay was tested using the simple lateral flow readouts. A high agreement was observed between the fluorescent-based detection and the lateral flow readout results (FIG. 3D-E)

SARS-CoV-2 Detection Via RT-LAMP Coupled CRISPR-Cas12b

The disclosed two-pot iSCAN systems, employing RT-LAMP coupled with CRISPR-Cas12a, reliably and specifically detected SARS-CoV-2 from clinical patient samples. However, developing virus detection modalities suitable for POC testing might require minimal liquid handling and single-pot reactions to facilitate wide adoption and in-field deployment. Therefore, additional studies aimed to further simplify the iSCAN system and develop a one-pot assay by employing the thermophilic variants of Cas12b that can catalytically function in the same temperature range as RT-LAMP. To this end, Cas12b was purified from Alicyclobacillus acidoterrestris (AacCas12b) and Cas12b from Alicyclobacillus acidophilus (AapCas12b) variants [38, 39]. The cis activity of these proteins was confirmed on PCR products of the target sequences using sgRNA sequence of the AacCas12b variant. The data show that both Cas12b variants were capable of inducing double-strand breaks using the same sgRNA at 62° C., and had robust collateral activities when incubated with ssDNA FQ reporter (FIG. 3F-G). However, because AapCas12b showed enhanced cis cleavage activity at 62° C., it was selected for use in further experiments.

The goal of utilizing the thermophilic Cas12b variants was to develop a simple one-pot and single temperature detection modality that minimizes liquid handling. This can be achieved by mixing all amplification and CRISPR-based detection reagents simultaneously in one pot, which allows simultaneous target amplification and detection. Thus, using the N gene assay, studies were conducted to verify the feasibility of performing SARS-CoV-2 detection in a one-pot assay format using the synthetic virus RNA in one-pot detection reaction mixtures containing AapCas12b, sgRNA, ssDNA-reporters, and RT-LAMP amplification reagents, and incubated the reaction mixtures at 62° C. for 1 h. The one-pot detection system was capable of detecting the viral RNA, albeit at lower efficiency compared to the two-pot system Figure data not shown). When the RT-LAMP product from one-pot assay was visualized on an agarose gel, weak amplification of the target virus RNA was seen. Therefore, it appeared that the concurrent presence of the active Cas12b-sgRNA complex in the same pot may lead to the digestion of the initial RT-LAMP product, which significantly affects the performance of the RT-LAMP amplification, and thus the robustness of the detection.

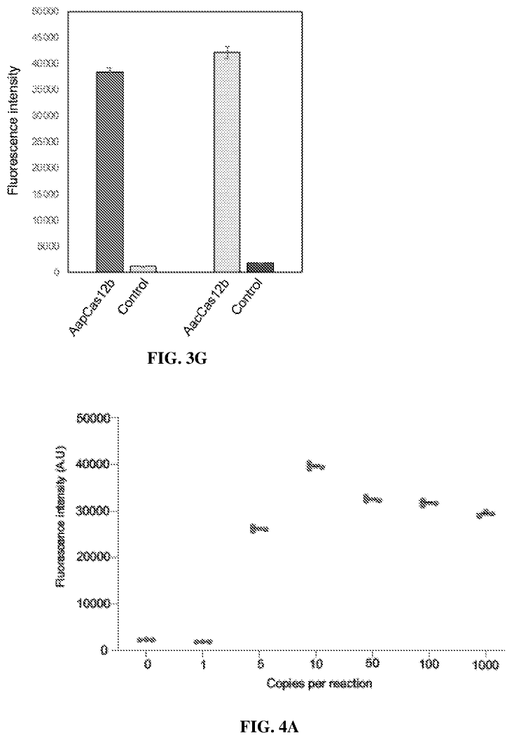

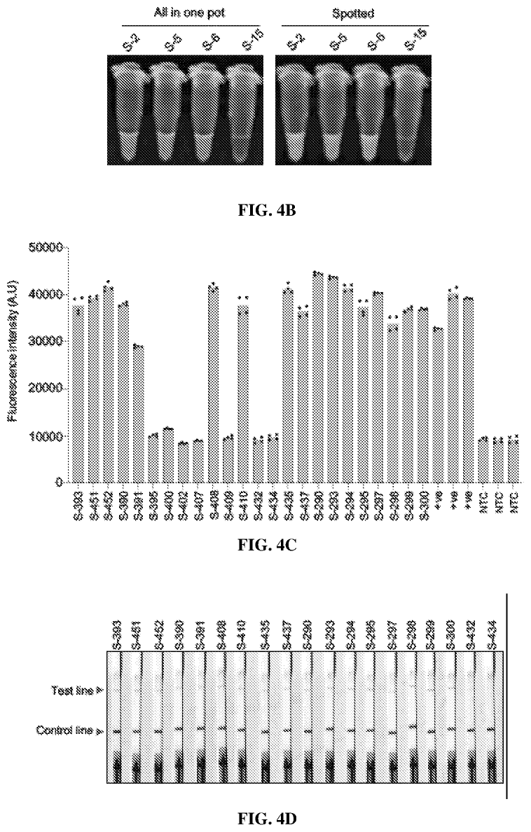

To improve the performance of the one-pot detection, the Cas12b enzyme was separated from the rest of the detection components by adding the Cas12b protein in a droplet on the tube wall, then allowing the RT-LAMP reaction to proceed before mixing the Cas12b with the other reaction components. An enhanced RT-LAMP amplification of the target and improved detection performance was observed using this “spotted” one-pot reaction with synthetic viral RNA. In the spotted one-pot reaction, AapCas12b consistently achieved a LoD of 10 copies per reaction (FIG. 4A and data not shown).

Next, the ability of the AapCas12b-based detection system to detect SARS-CoV-2 in clinical samples (3 positive and 1 negative) using fluorescence-based readouts was tested. AapCas12b was capable of detecting the SARS-CoV-2 RNA using spotted one-pot and all-mixed one-pot reactions (FIG. 4B). Using the spotted one-pot AapCas12b detection system, the 24 clinical samples used with the Cas12a based detection system were tested. The performance of AapCas12b system was very similar to the performance of Cas12a-based system, where 18 out of the 21 qPCR-positive samples showed a positive signal, and the 3 qPCR negative samples showed negative results using fluorescence-based readouts (FIG. 4C). However, when the system was tested on 17 of the positive samples and 2 negative samples using lateral flow readouts, we consistently observed weak performance measured by the weak signal on lateral flow readouts in positive samples compared to negative ones, which compromised the interpretation of the results (FIG. 4D).

Discussion

The COVID-19 outbreak poses an unprecedented public health challenge worldwide. The crucial need for the large-scale detection of SARS-CoV-2 has made it urgent to develop local solutions for creating sensitive, specific, low-cost, in-field deployable diagnostic kits. The bio-manufacturing of assay reagents and simple portable machines for easy use at POC will facilitate the widespread, large-scale detection of this virus. In this study, iSCAN, a system involving RT-LAMP coupled with CRISPR-Cas12, was established as an efficient detection module for COVID-19. Different sets of LAMP primers were designed, screened and tested leading to identification of a few primer sets for specifically and efficiently amplifying the SARS-CoV-2 E and N genes. Due to huge disruptions in the supply chain worldwide, the RTx and BstI enzymes were employed for the RT-LAMP reactions.

The addition of CRISPR reagents provides important specificity to the assay. Although the results of LAMP reactions can be visualized with a pH-sensitive dye upon DNA amplification, false amplification, cross-contamination, and the formation of primer-dimers may lead to false signals [22, 23]. Therefore, a specificity factor is needed to ascertain the identity of the amplification products and whether these products are derived from virus sequences. Because CRISPR-Cas12 in the disclosed system cuts DNA only in the presence of the SARS-CoV-2 sequence, the Cas12 enzyme serves as a specificity factor for RT-LAMP. RT-LAMP polymerases can be used for reverse transcription of the viral genome and subsequent amplification, producing a sufficient DNA template for CRISPR-Cas12 to facilitate complete virus detection in 1 h in one-pot or two-pot assays, depending on the Cas12 variant employed in the detection system. To ensure that the amplified RT-LAMP products are derived from genuine virus sequences, RT-LAMP and CRISPR-Cas12-based detection were coupled. RT-LAMP coupled with CRISPR-Cas12 offers a binary result of RT-LAMP and Cas12 activity, providing a highly specific, robust virus detection modality for SARS-CoV-2.

The LoD of the iSCAN system is comparable with the minimum level required to detect the virus in clinical samples by qRT-PCR Minimal sample processing is sufficient for the iSCAN system to detect SARS-CoV-2, enabling large-scale screening. Our iSCAN system for SARS-CoV-2 detection is portable and can easily be deployed in the field due to its simplicity and low cost (2-5 USD/reaction).

The disclosed iSCAN assays have been adjusted to make them amenable to use by lay users without specialized equipment, but have retained some complexity to assure accurate assays. For example, although one-pot detection systems are favored over two-pot detection systems for onsite detection, the present data show that mixing various components in one pot leads to a substantial reduction in detection performance, specificity, and sensitivity.

The iSCAN system can be further simplified through the use of cellular extracts harboring assay reagents to maximize product integrity during shipping and handling. This would allow a lay user to simply mix, incubate, and vortex the sample and visualize the assay results. The large-scale production of these reagents in various forms is feasible, including bacterial extracts or freeze-dried cells, allowing large-scale in-field deployment in low resource environments [40]. The calorimetric visualization of the test results does not require special equipment and can be conducted by the naked eye or using a simple portable machine. Manifolds for both the RT-LAMP and CRISPR-Cas12 reactions could be designed to facilitate sample processing, reaction mixing, and assay results to be obtained in a closed system to allow non-healthcare staff to perform these assays.

In addition to providing high specificity, the iSCAN assays can be easily customized as the virus mutates. The iSCAN two-pot and one-pot systems using RT-LAMP coupled with CRISPR-Cas12 allow SARS-CoV-2 to be visually detected with a detection limit comparable to that of qRT-PCR. This detection module couples the robustness of the RT-LAMP assay with the specificity of CRISPR-Cas12, resulting in an efficient and reproducible detection system. Because new mutations could occur in the current virus strain, iSCAN can easily be tailored to detect any virus strain and to differentiate between different strains and determine the dominance of a specific strain in a certain location or population.

Moreover, the iSCAN assays can be used for high-throughput testing. Various detection modules, including GeneXpert, Cephied, and ID NOW by Abbot are low throughput and require the use of a single machine for each sample, which may be impractical for low-resource environments and where large-scale testing in a short time window is needed. Because asymptomatic virus transmission has been reported, and initially negative results may not be conclusive, regular testing may be required for patients with symptoms and their asymptomatic primary and secondary contacts. The iSCAN system is suitable for conducting multiple consecutive tests at the POC. Because the viral load in a patient plays a significant role in the assay results, regular testing is required. The development of the iSCAN system as a POC SARS-CoV-2 testing modality would enable affordable and regular testing, which is crucial for reducing false negatives and identifying clusters of infection as early as they arise. To facilitate sample collection and testing, a patient's saliva could be used for virus detection [41], and the iSCAN module could be developed as an in-home SARS-CoV-2 testing kit. The iSCAN detection platform can differentiate between coronaviruses and identify specific SARS-CoV-2 strains. Therefore, the iSCAN detection system could greatly facilitate the effective management and control of virus spread, specifically in high-risk areas, including clinics, airports, and low resource areas where the test could be conducted and results obtained in a short time.

The iSCAN detection module might not require a nucleic acid extraction step and may be compatible with different types of samples, including saliva and nasal swabs. Furthermore, quick nucleic acid extraction could be coupled with iSCAN to speed up the detection and bypass the need to conduct an RNA extraction step. iSCAN could be used to assess patient-collected saliva samples in a tube with extraction buffer that is compatible with RT-LAMP and CRISPR-Cas12 reactions. The iSCAN detection module will ensure the safety of both healthcare personnel and laboratory staff.

Other versions of iSCAN could be developed in the future for multiplex targeting of different virus strains and the simultaneous identification of other pathogens. Although we have shown that iSCAN works quite efficiently with lateral flow cells, inexpensive visualizers of the reporters could be employed to increase the throughput of the assays and more rapidly produce results from a large number of samples. Furthermore, this represents an effective alternative should the supply of lateral flow cells become limited or disrupted. Other DIY modalities could be developed to couple the assay reactions and test results with a smartphone to allow the results to be communicated rapidly. In conclusion, the iSCAN virus detection platform should enable robust testing on a massive scale in low-resource environments, thereby helping to mitigate the transmission and spread of SARS-CoV-2 and providing effective management of the COVID-19 pandemic.

### Example 2: Development of a Cas13-Based, One-Pot Assay for SARS-CoV-2 Detection

This Example reports the identification and characterization of a thermostable Cas13a ortholog from Thermoclostridium caenicola (TccCas13a) that is highly active at high temperatures required for some amplification methods, such as RT-LAMP. Besides TccCas13a, other thermophilic Cas13a orthologues, including Cas13a from Herbinix hemicellulosilytica (HheCas13a) also exhibited strong activity at elevated temperatures. These Cas13s were characterized with regards to temperature, reporter cleavage preference and optimal reaction conditions. RT-LAMP utilizing a SARS-CoV-2 N-gene specific primer set with FIP primer carrying a T7 promoter was coupled to in vitro transcription mediated by the thermostable Hi-T7 RNA polymerase and the reaction conditions were optimized for one-pot detection assay. The data described below demonstrates that this protocol can robustly detect SARS-CoV-2 in clinical samples derived from COVID-19 patients. The data indicate that the thermophilic Cas13-based one-pot detection assay holds great promise for use as a prospective POC modality to help address increased demand for SARS-CoV-2 testing.

Materials and Methods

Computational Identification of a Thermophilic CRISPR/Cas13a

Various existing Cas13 enzymes and their bacterial hosts were manually interrogated to identify potential thermophilic Cas13s originating from thermophilic organisms. After the identification of HheCas13a as a potential thermophilic Cas13 protein, the protein sequence of HheCas13a was used as a query in the Basic Local Alignment Search Tool (BLAST) against the NCBI non-redundant (nr) protein database using default settings. Only subject sequences with query coverage (Query cover) above 80% were considered for second round of host interrogation (analyzing growth conditions using BacDive data base and other resources). TccCas13a protein (accession #WP_149678719.1) from Thermoclostridium caenicola was identified as a potential thermophilic Cas13 protein.

Phylogenetic tree was reconstructed using protein sequences of different Cas13s belonging to different families/subtypes of Class II/type VI CRISPR-Cas systems. All protein sequences were organized in a single .txt file and aligned using MUSCLE in MEGAX software with default settings. The phylogenetic reconstruction was based on the Maximum-Likelihood method using MEGAX with WAG+G+F model and 1000 bootstrap samplings. The generated output file (.nwk) was visualized using TreeGraph_2.

CRISPRCasFinder [1] was performed on the genomic DNA sequence (GenBank #NZ_FQZP01000023.1) to identify the associated CRISPR array. CRISPRDetect [2] was then used to predict the orientation of the direct repeat in the TccCas13a CRISPR array.

Cas13 Protein Expression and Purification

The expression vector for HheCas13a “p2CT-His-MBP-Hhe_Cas13a_WT” was obtained from Addgene (plasmid #91871), and the purification of HheCas13a was performed following a previously published protocol [55]. To produce the expression plasmid for TccCas13a expression and purification, the E. coli codon-optimized TccCas13 coding sequence was synthesized (GenScript) de novo and subcloned in frame with His and SUMO tags on the N-terminus into the His6-TwinStrep-SUMO bacterial expression vector (Addgene #115267) using BamHI and NotI (Table 8).

Purification of TccCas13a protein was performed following the protocol of Kellner et al. (2019) [65] with a few modifications. Briefly, the TccCas13a expression vector was transformed into BL21 E. coli cells. Starter cultures were prepared by growing single colonies in LB broth supplemented with 100 μg/mL ampicillin for around 12 h at 37° C. Next, 25 mL of starter culture was used to inoculate 1 L of Terrific Broth medium (TB) (IBI scientific) supplemented with 100 μg/mL ampicillin, and the 1 L cultures (4 L total) were incubated at 37° C. until an OD₆₀₀ of 0.5. Cells were incubated at 4° C. 30 mins, and the expression was induced with 0.5 mM IPTG. Cultures were then incubated at 16° C. at 180 rpm for overnight expression. Next, cells were harvested by centrifugation for 20 min at 4° C. at 4000 rpm. Cell pellets were resuspended in lysis buffer (50 mM Tris-Cl pH 7.5, 300 mM NaCl, 5% glycerol, 1 mM TCEP, 4.5 mM MgCl2, 1 mM PMSF, EDTA-free protease inhibitor (Roche)) and supplemented with 1 mg/mL lysozyme (L6876, Sigma). Cells were then lysed by sonication and clarified by centrifugation at 12,000 rpm for 60 min. The soluble 6×His-SUMO-TccCas13a in cleared lysate was then purified with an affinity chromatography column (HiTrap Q HP, 5 mL GE Healthcare) (AKTA PURE, GE Healthcare) followed by concurrent removal of the 6×His-SUMO tag by SUMO protease and overnight dialysis in dialysis buffer (50 mM Tris-HCL pH 7.5, 200 mM KCL, 5% glycerol, 1 mM TCEP). Cleaved protein was concentrated to 1.5 mL by Amicon Ultra-15 Centrifugal Filter Units (100 kDa NMWL, UFC905024, Millipore) and further purified via size-exclusion chromatography on a 5200 column (GE Healthcare) in gel filtration buffer (50 mM Tris-HCl, 200 mM KCL, 10% glycerol, 1 mM TCEP, pH 7.5). The protein-containing fractions resulting from the gel filtration were pooled, snap frozen, and stored at −80° C.

Nucleic Acid Preparation

A short region of the SARS-CoV-2 N gene sequence was used as the target sequence in all preliminary thermophilic Cas13 characterization and optimization experiments to screen collateral reporters and assess thermostability of Cas13 proteins. The N gene target RNA sequence was prepared by in vitro transcription of PCR amplicons containing the T7 promoter sequence using the 2019-nCoV_N_Positive Control plasmid as a PCR template (10006625, IDT) (primers used are listed in table 2). Purified PCR amplicons (QIAquick PCR Purification Kit, QIAGEN) were transcribed in vitro using HiScribe T7 Quick High Yield RNA Synthesis Kit (E2050, NEB). The transcripts were then purified by using Direct-zol RNA Miniprep Kits (R2050, Zymo Research) following the manufacturer's instructions, and the purified RNA was stored at −80° C.

HheCas13a and TccCas13a crRNAs were designed to target the N gene sequence of the SARS-CoV-2 genome. For crRNA production, templates for in vitro transcription were generated using single-stranded DNA oligos containing a T7 promoter, scaffold, and spacer in reverse complement orientation (IDT), and were then annealed to T7 forward primer in Taq DNA polymerase buffer (Invitrogen) (Table 9). The annealed oligos were then used as templates for the subsequent in vitro transcription as described above.

To establish the thermophilic Cas13-based one-pot assay, control synthetic SARS-CoV-2 viral genomic sequences were ordered as synthetic RNA from Twist Bioscience, and were diluted to 10,000 RNA copies/μL and used at indicated concentrations. For RT-LAMP amplification (described below), previously published LAMP primers designed to amplify the SARS-CoV-2 N gene (Joung et al., 2020 [57], Broughton et al., 2020 [49]) were used, with the following modifications. The FIP or BIP primers were designed with the T7 promoter sequence appended at the 5′ end of the first half of the primers (Table 5).

Differential Scanning Fluorimetry

DSF was performed using 5 to 15 uM of the purified Cas13 proteins in gel filtration buffer (with 5% glycerol) containing 10% SYPRO Orange fluorescent dye (ThermoFisher, 56650) in final reaction volume of 35 uL. Proteins were tested in triplicates and the fluorescence was monitored using a 96-well Real-Time PCR detection system (CFX96 qPCR machine, Bio-Rad), from 25 to 95° C., with a gradual temperature increase of 1° C. every 10 s.

Protein Thermostability Assay

LwaCas13a, HheCas13a and TccCas13a proteins were diluted to approximately 0.2 mg/mL in protein storage buffer (50 mM Tris-HCL pH 7.5, 600 mM NaCl, 5% glycerol, 2 mM DTT) and incubated at a range of temperatures (37, 60, 70 and 90° C.) for 30 minutes. Samples were spun down in a microcentrifuge at 14200 rpm for 25 minutes. A total of 5 μL of the supernatant was mixed with the same volume of protein sample loading buffer and heated at 95° C. for 10 minutes. The samples were cooled on ice for 3 minutes and run on NuPAGE (10%) Bis-Tris polyacrylamide gel (ThermoFisher, NP0301BOX).

Protein thermostability assay for HheCas13a and TccCas13a RNPs was performed in the same way with the exception of incubating the aforementioned proteins with 1 μM of their cognate crRNAs for 5 minutes at 37° C. in order to assemble the RNP before subjecting them to a range of different temperatures.

In Vitro Cis Cleavage Assays

HheCas13a and TccCas13a cleavage reactions were performed at 37° C. and 60° C. with synthetic, in vitro transcribed RNA target. Briefly, for both HheCas13a or TccCas13a cleavage assays, cleavage reactions were carried out in 20 μL reaction volume with 50 nM of Cas13a protein, 50 nM of their cognate crRNAs, and 100 nM of target RNA in 1× isothermal buffer (20 mM Tris-HCl, 50 mM KCl, 10 mM (NH₄)₂SO₄, 2 mM MgSO4, 0.1% Tween 20, pH 8.8) supplemented with additional 6 mM MgSo4 (final of 8 mM MgSo2), and the reactions were then incubated at the indicated temperatures for 1 hr (no pre-assembly of Cas13a protein and crRNA to form RNP was performed). The samples were then boiled at 70° C. for 3 minutes in 2×RNA Loading Dye (NEB, B0363S) and cooled on ice for 3 minutes before loading into 6% polyacrylamide-urea denaturing gel. The electrophoresis was run for 45 minutes at 25W. The gel was stained with SYBR™ Gold Nucleic Acid Gel Stain (ThermoFisher, S11494) for 10 minutes, briefly washed with 1×TBE buffer and visualized using Bio-Rad Molecular Imager® Gel Doc™ system.

Fluorescent ssRNA Cleavage Assays

For all reporter screening and thermostability assays, 50 nM of HheCas13a or TccCas13a proteins were incubated with 50 nM of the respective crRNAs, 250 nM of ssRNA reporter (either poly A, poly U, or mixed sequence reporter) in 1×isothermal buffer (20 mM Tris-HCl, 50 mM KCl, 10 mM (NH₄)₂SO₄, 2 mM MgSO4, 0.1% Tween 20, pH 8.8) supplemented with additional 6 mM MgSo4 (final of 8 mM MgSo2), 0.8 U/uL RNaseOUT (10777019, Invitrogen) and 2 uL of (1-100 nM) of target RNA in 20 uL reaction volume. These reactions were incubated in a 96 well-plate (BioRad) at different temperatures for 1 hour in a PCR (C1000 touch thermal cycler, BioRad) machine using the FAM channel, with fluorescence measurements taken every 2 min.

One-Pot Detection Reactions

For Bst DNA polymerase screening and other optimization reactions, reverse transcription and LAMP isothermal amplification of the target nucleic acids, coupled with T7-mediated in vitro transcription and Cas13-based detection of the amplified and in vitro-transcribed target RNA was carried out in the same tube. The reaction was performed using RT-LAMP primers with final concentrations of 1.6 μM FIP/BIP primers (with the T7 promoter sequence fused to either the FIP or BIP primer), 0.2 μM F3/B3 primers, and 0.4 μM LF/LB primers, 1×Isothermal Amplification Buffer (from different vendors in Bst DNA polymerase screening reactions) or from Lucigen (30027, Lucigen) in other optimization experiments), 1.4 mM dNTPs, 0.32 U/uL Bst DNA Polymerase (from different vendors in Bst DNA polymerase screening reactions) or from Lucigen (30027, Lucigen)), 0.45 U/uL of WarmStart RTx Reverse Transcriptase (M0380, NEB) or 2 U/uL SuperScript IV reverse transcriptase (18090010, Invitrogen), 6 mM MgSO4, 0.1 U/uL thermostable RNAseH (M0523S, NEB), 0.8 U/uL RNaseOUT or RNasin plus (N2611, Promega), 0.5 mM NTPS, 4 U/μL Hi-T7 RNA polymerase (M0658S, NEB), 0.4 U/μL thermostable inorganic pyrophosphatase (M0296, NEB), 250 nM RNA reporter, 50 nM Cas13, 50 nM crRNA, and 2 μL of template RNA in 25-4, reactions. These reactions were incubated in a 96 well-plate (BioRad) at 56° C. for 1-2 hours in a PCR (C1000 touch thermal cycler, BioRad) machine using the FAM channel, with fluorescence measurements taken every 2 min

### Nucleotide and Amino Acid Sequences Used

TABLE 5
RT-LAMP primers used in Examples 2 and 3.

| Name | Sequence | Note |
|---|---|---|
| DE-F3 | GAAATTTGGATCTTTGTCATCC (SEQ ID NO: 29) | Original and |
| DE-B3 | GAAATTTGGATCTTTGTCATCC (SEQ ID NO: 30) | modified |
| DE-FIP | TGCGGCCAATGTTTGTAATCAGCCAAGGAAATTTTGG | DETECTR |
| | GGAC (SEQ ID NO: 31) | primers. |
| DE-BIP | CGCATTGGCATGGAAGTCACTTTGATGGCACCTGTGT | Modified part (T7 |
| | AG (SEQ ID NO: 32) | promoter |
| DE-LF | TTCCTTGTCTGATTAGTTC (SEQ ID NO: 33) | sequence) shown |
| DE-LB | ACCTTCGGGAACGTGGTT (SEQ ID NO: 34) | in underline. |
| DE-FIP- | TGCGGCCAATGTTTGTAATCAGTAATACGACTCACTA | |
| T7 | TAGGCCAAGGAAATTTTGGGGAC (SEQ ID NO: 35) | |
| DE- | CGCATTGGCATGGAAGTTAATACGACTCACTATAGGC | |
| BIP-T7 | ACTTTGATGGCACCTGTGTAG (SEQ ID NO: 36) | |
| SC-F3 | GCTGCTGAGGCTTCTAAG (SEQ ID NO: 37) | Original and |
| SC-B3 | GCGTCAATATGCTTATTCAGC (SEQ ID NO: 38) | modified |
| SC-FIP | GCGGCCAATGTTTGTAATCAGTAGACGTGGTCCAGAA | STOPCovid |
| | CAA (SEQ ID NO: 39) | primers. |
| SC-BIP | TCAGCGTTCTTCGGAATGTCGCTGTGTAGGTCAACCA | Modified part (T7 |
| | CG (SEQ ID NO: 40) | promoter |
| SC-LF | CCTTGTCTGATTAGTTCCTGGT (SEQ ID NO: 41) | sequence) shown |
| SC-LB | TGGCATGGAAGTCACACC (SEQ ID NO: 42) | in underline. |
| SC-T7- | GCGGCCAATGTTTGTAATCAGTTAATACGACTCACTAT | |
| FIP | AGGGAGACGTGGTCCAGAACAA (SEQ ID NO: 43) | |
| SC-T7- | TCAGCGTTCTTCGGAATGTCGCTAATACGACTCACTAT | |
| BIP | AGGGTGTGTAGGTCAACCACG (SEQ ID NO: 44) | |

SEQ ID NOs: 37-44 were used in Example 2. SEQ ID NOs: 29-44 were used in Example 3.

TABLE 6
Primers to PCR amplify N gene regions for IVT in
Examples 2 and 3.

| | |
|---|---|
| N gene-1- | GAAATTAATACGACTCACTATAGGGAGGCTTCTAAGAAGC |
| T7-F | CTCGGC (SEQ ID NO: 45) |
| N gene-1- | CTGTCTCTGCGGTAAGGCTTGAG (SEQ ID NO: 46) |
| R_2 | |
| N gene- | GAAATTAATACGACTCACTATAGGGTTGCAACTGAGGGAG |
| target 2- | CCTTG (SEQ ID NO: 70) |
| T7-F | |
| N gene- | GCTTCTTAGAAGCCTCAGCAGCAG (SEQ ID NO: 71) |
| target-2- | |
| R | |

TABLE 7
RNA reporter designs and sequences used in
Examples 2 and 3.

| | |
|---|---|
| UA reporter-FAM | 5′-/56-FAM/UUAUU/3IABKFQ/-3′ |
| UA reporter-HEX | 5′-/HEX/UUAUU/3IABKFQ/-3′ |
| Poly U reporter-FAM | 5′-/56-FAM/UUUUU/3IABKFQ/-3′ |
| Poly A reporter-FAM | 5′-/56-FAM/AAAAA/3IABKFQ/-3′ |
| Mix reporter-FAM | 5′-/56-FAM/UGACGU/3IABKFQ/-3′ |

TABLE 8
Cas13 protein sequence used in this study.

| Name | Protein sequence |
|---|---|
| TccCas13a | MGSSHHHHHHSSGLVPRGSHMASWSHPQFEKGGGSGGGSGGSA |
| | WSHPQFEKMSDSEVNQEAKPEVKPEVKPETHINLKVSDGSSEIFFK |
| | IKKTTPLRRLMEAFAKRQGKEMDSLRFLYDGIRIQADQTPEDLDM |
| | EDNDIIEAHREQIGGSMKITKRKWGEHHPPLYFYRDEDSGRLLAQ |
| | NDRKQDYTDTLFNDIAQDTFERSLRNRLLKTPEKGDKRFYSNEIV |
| | KLVEKLCQGADVAEIMKSMERNEKLRPKNEKEIKNLKKQLDGTL |
| | SEYGKRYTAPEGAMTLNDALFYLVEGNPLKQAMAKAELGKIREA |
| | LIKEKENRINRVRYSIKNNKIPLRIQEDGGITPNNDRAAWLLGLMK |
| | PADPAKGITDCYPLLGELEEVFDFDKLSKTLHEKISRCQGRPRSIA |
| | MAVDEALKQYLRELWEKSPSRQQDLKYYFQAVQEYFKDNFPIRT |
| | KRMGARLRQELLKDKTSLSRLLEPKHMANAVRRRLINQSTQMHI |
| | LYGKLYAYCCGEDGRLLVNSETLQRIQVHEAVKKQAMTAVLWSI |
| | SRLRYFYQFEDGDILSNKNPIKDFRDKFLRDTNKYTHEDVEACKE |
| | KLQDFFPLKELQEKIKEDAKGLQETDNKQADTTDFKAIGHIVRDD |
| | RKLCNQLLAECVSCIGELRHHIFHYKNVTLIQALKRIADKVKPEDL |
| | SVLRAIYLLDRRNLKKAFAKRISSMNLPLYYREDLLSRIFKKEGTA |
| | FFLYSAKIQMTPSFQRVYERGKNLRREFECERMKAEASNGQNGQ |
| | DGDRLKWFRQLAAGDSADTHFNWAVEAYAESAADVENNVEFDT |
| | DVDAQRALRNLLLLIYRHHFLPEVQKDETLVTGKIHKVLERNRQL |
| | SEGQGPNQGKAHGYSVIEELYHEGMPLSDLMKQLQRRISETERES |
| | RELAQEKTDYAQRFILDIFAEAFNDFLEAHYGEEYLEIMSPRKDAE |
| | AAKKWVKESKTVDLKTSIDEKEPEGHLLVLYPVLRLLDERELGEL |
| | QQQMIRYRTSLASWQGESNFSEEIRIAGQIEELTELVKLTEPEPQFA |
| | EEVWGKRAKEAFEDFIEGNMKNYEAFYLQSDNNTPVYRRNMSR |
| | LLRSGLMGVYQKVLASHKQALKRDYLLWSEKHWNVKDENGADI |
| | SSAEQAQCLLQRLHRKYAESPSRFTEEDCKLYEKVLRRLEDYNQA |
| | VKNLSFSSLYEICVLNLEILSRWVGFVQDWERDMYFLLLAWVRQ |
| | GKLDGIKEEDVRDIFSEGNIIRNLVDTLKGENMNAFESVYFPENKG |
| | SKYLGVRNDVAHLDLMRKNGWRLEAGKTCSVMEDYINRLRFLL |
| | SYDQKRMNAVTKTLQQIFDRHKVKIRFTVEKGGMLKIEDVTADK |
| | IVHLKGSRLSGIEIPSHGERFIDTLKALMVYPRG* (SEQ ID NO: 47) |

6x His affinity tag: residues 5-10; Thrombin site: residues 14-19; Strep-tag II: residues 24-31 and 44-51; SUMO: residues 52-148; TccCas13a protein: residues 151-1375. For HheCas13 sequence (Ref[55]).

TABLE 9
crRNA sequences used in this study.

| crRNA | | |
|---|---|---|
| # | crRNA Name | crRNA sequence |
| 1101 | Hhe-Sense-28-1 | ATCAGACAAGGAACTGATTACAAACATTGTTGC |
| | | AGTTCCCCTGTCTACGGGGATTGTTACCCTATAG |
| | | TGAGTCGTATTAATTTC |
| | | (SEQ ID NO: 48) |
| 1102 | Hhe-Sense-28-2 | CCGCAAATTGCACAATTTGCCCCCAGCGGTTGC |
| | | AGTTCCCCTGTCTACGGGGATTGTTACCCTATAG |
| | | TGAGTCGTATTAATTTC |
| | | (SEQ ID NO: 49) |
| 1103 | Hhe-Sense-28-3 | CGCTTCAGCGTTCTTCGGAATGTCGCGCGTTGCA |
| | | GTTCCCCTGTCTACGGGGATTGTTACCCTATAGT |
| | | GAGTCGTATTAATTTC |
| | | (SEQ ID NO: 50) |
| 1104 | Hhe-Sense-28-4 | CATTGGCATGGAAGTCACACCTTCGGGAGTTGC |
| | | AGTTCCCCTGTCTACGGGGATTGTTACCCTATAG |
| | | TGAGTCGTATTAATTTC (SEQ ID NO: 51) |
| 1105 | Hhe-Sense-28-5 | GAACGTGGTTGACCTACACAGGTGCCATGTTGC |
| | | AGTTCCCCTGTCTACGGGGATTGTTACCCTATAG |
| | | TGAGTCGTATTAATTTC |
| | | (SEQ ID NO: 52) |
| 1106 | Hhe-Sense-28-6 | ATCAAATTGGATGACAAAGATCCAAATTGTTGC |
| | | AGTTCCCCTGTCTACGGGGATTGTTACCCTATAG |
| | | TGAGTCGTATTAATTTC (SEQ ID NO: 53) |
| NS | Hhe-NS | ctccgtgatatggctcttcgcatgctgaGTTGCAGTTCCCCTGTC |
| | | TACGGGGATTGTTACCCTATAGTGAGTCGTATTA |
| | | ATTTC (SEQ ID NO: 54) |
| 1087 | Hhe-AntiSense- | TCCCGAAGGTGTGACTTCCATGCCAATGGTTGC |
| | 28-4 | AGTTCCCCTGTCTACGGGGATTGTTACCCTATAG |
| | | TGAGTCGTATTAATTTC (SEQ ID NO: 55) |
| 1088 | Hhe-AntiSense- | AATTTGGATCTTTGTCATCCAATTTGATGTTGCA |
| | 28-6 | GTTCCCCTGTCTACGGGGATTGTTACCCTATAGT |
| | | GAGTCGTATTAATTTC (SEQ ID NO: 56) |
| 1125 | Tcc-Sense-24-1 | CAGACAAGGAACTGATTACAAACAGTTGCAGTC |
| | | TCCGCCTACATGGGAGTTGTGACCCTATAGTGA |
| | | GTCGTATTAATTTC (SEQ ID NO: 57) |
| 1126 | Tcc-Sense-24-3 | CTTCAGCGTTCTTCGGAATGTCGCGTTGCAGTCT |
| | | CCGCCTACATGGGAGTTGTGACCCTATAGTGAG |
| | | TCGTATTAATTTC (SEQ ID NO: 58) |
| 1127 | Tcc-Sense-24-6 | CAAATTGGATGACAAAGATCCAAAGTTGCAGTC |
| | | TCCGCCTACATGGGAGTTGTGACCCTATAGTGA |
| | | GTCGTATTAATTTC (SEQ ID NO: 59) |
| 1171 | Tcc-Sense-24-2 | GCAAATTGCACAATTTGCCCCCAGGTTGCAGTCT |
| | | CCGCCTACATGGGAGTTGTGACCCTATAGTGAG |
| | | TCGTATTAATTTC (SEQ ID NO: 60) |
| 1172 | Tcc-Sense-24-4 | TTGGCATGGAAGTCACACCTTCGGGTTGCAGTCT |
| | | CCGCCTACATGGGAGTTGTGACCCTATAGTGAG |
| | | TCGTATTAATTTC (SEQ ID NO: 61) |
| 1173 | Tcc-Sense-24-5 | ACGTGGTTGACCTACACAGGTGCCGTTGCAGTC |
| | | TCCGCCTACATGGGAGTTGTGACCCTATAGTGA |
| | | GTCGTATTAATTTC (SEQ ID NO: 62) |
| NS | Tcc-NS | actcagccatccacatccgagtcttcagGTTGCAGTCTCCGCCTA |
| | | CATGGGAGTTGTGACCCTATAGTGAGTCGTATTA |
| | | ATTTC (SEQ ID NO: 63) |
| 1174 | Tcc-AntiSense- | TGTTTGTAATCAGTTCCTTGTCTGGTTGCAGTCT |
| | 24-1 | CCGCCTACATGGGAGTTGTGACCCTATAGTGAG |
| | | TCGTATTAATTTC (SEQ ID NO: 64) |
| 1175 | Tcc-AntiSense- | GCGACATTCCGAAGAACGCTGAAGGTTGCAGTC |
| | 24-3 | TCCGCCTACATGGGAGTTGTGACCCTATAGTGA |
| | | GTCGTATTAATTTC (SEQ ID NO: 65) |
| T7 oligo | T7 oligo for IVT | GAAATTAATACGACTCACTATAGGG (SEQ ID |
| | | NO: 66) |

crRNA sequences shown as 5′→3 reverse complement to be annealed with T7 oligo for IVT.

TABLE 10
Clinical samples used in this study.

| Sample number | Sample ID | Ct value |
|---|---|---|
| 391 | 160935 | 16 |
| 392 | 160938 | 14 |
| 393 | 160939 | 16 |
| 394 | 160941 | 16 |
| 395 | 160942 | 27 |
| 396 | 160943 | 14 |
| 397 | 160944 | 20 |
| 398 | 160947 | 14 |

Results

Reverse transcription loop-mediated isothermal amplification (RT-LAMP) is a highly sensitive, robust and practical method that holds great promise in developing a viable POC detection platform. However, when used independently as a method to detect nucleic acids, it suffers from a high rate of false positives due to primer-dimer formation and cross-contamination. In order to address these drawbacks, introducing additional level of specificity is highly desirable. Coupling RT-LAMP to specific CRISPR/Cas target recognition and in trans reporter cleavage ensures signal appearance only in reactions where a correct amplicon has been generated. Nevertheless, most methods reported to date rely on transferring RT-LAMP product to the second tube, which complicates handling and, more importantly, leads to aerosol formation that is detrimental to accuracy of subsequent reactions. In order to address these shortcomings, an assay where all components are added in a single step and the reaction is incubated at a single temperature is needed. To achieve this goal, thermostable reagents are needed that can tolerate the relatively high temperatures needed for the RT-LAMP reactions.

Identification, Screening and Characterization of Thermophilic Cas13 Enzymes

Recently, Cas13 variants from mesophilic bacteria have been employed for biosensing, including pathogen detection, genotyping, and diagnostics of viruses and disease markers. The SARS-CoV-2 pandemic highlighted the need to develop POC diagnostics. RT-LAMP is a practical approach for POC diagnostics. Still, it needs to be developed in a one-pot closed system to avoid cross-contamination, facilitate user-friendliness and enhance sensitivity and specificity. The current Cas13 homologs can only be used in a two-pot assay which is problematic and poses a risk of cross-contamination. Therefore, it was aimed to identify Cas13 proteins from thermophilic bacteria and test their thermostability in a wider temperature range especially at higher temperatures suitable for developing one-pot RT-LAMP assay involving virus genome amplification and CRISPR-mediated detection in a single tube.

Cas13 variants were interrogated to determine whether some of these originate from thermophilic hosts. HheCas13a originating from Herbinix hemicellulosilytica thermophilic bacterium was identified as a potential thermophilic protein. Subsequently, the HheCas13a (1285 amino acids) was used as a query in BLAST-P NCBI searches of non-redundant protein sequences datasets to interrogate databases for potential thermophilic Cas13 homologs. A TccCas13 homolog originating from Thermoclostridium caenicola, sharing 87% identity at the amino acid level, was identified a as another likely thermophilic protein. The gene sequence of TccCas13a was synthesized and the available clone of HheCas13a was used for heterologous expression in E. Coli followed by purifying the proteins to homogeneity. Subsequently, differential scanning Fluorimetry (DSF) was conducted to test the thermostability of the HheCas13 and TccCas13a proteins.

The data showed that both proteins possess a denaturation temperature higher than those of mesophilic bacteria, e.g., LwaCas13a. Next, it was hypothesized that the complexing of sgRNA and the HheCas13 and TccCas13 proteins would further stabilize the proteins at higher temperatures. In silico prediction of the TccCas13a crRNA was performed and the recently reported crRNA of the HheCas13a was used. TccCas13a and HheCas13a were incubated with and without their respective in vitro transcribed sgRNAs at 37, 60, 70 and 90° C. for 30 minutes. The SDS-PAGE analysis demonstrated that the TccCas13a and HheCas13a loaded with sgRNA exhibit higher thermostability. However, TccCas13a exhibited a better thermostability compared to HheCas13a.

The data showed that both proteins exhibit thermostability and thus are promising candidates for downstream applications requiring cis and trans catalytic activities at higher temperatures, e.g., one-pot RT-LAMP for diagnostic applications as well as targeted gene knockdown, virus interference, and RNA editing and imaging.

Characterization of the Cis and Trans Catalytic Activities of the TccCas13a and HheCas13a Thermophilic Proteins

The data showed that both proteins remain folded at higher temperatures and that loading of sgRNA enhances the thermostability of the proteins. Next, it was tested whether the RNP complex of these proteins is active and mediates cis and trans activities required for the downstream applications of these proteins. sgRNAs were designed to cleave a synthetic target sequence to determine the ability of both proteins to exhibit catalytic cis activities at higher temperatures.

The data showed that both proteins exhibit robust cis catalytic activities at higher temperatures.

In order to couple RT-LAMP with specific CRISPR/Cas based detection, two thermophilic Cas13 enzymes, namely HheCas13a and TccCas13a, were identified and their activity tested at relevant temperatures. When Cas13/crRNA ribonucleoproteins (RNP) recognize and cleave their target sequence, they exhibit collateral cleavage activity that degrades surrounding ssRNAs [53, 54]. Such collateral activity has been harnessed in nucleic acid detection applications, where a ssRNA probe (reporter) molecule present in the Cas13 reaction is degraded by target-dependent Cas13 collateral activity [52]. The ssRNA reporter contains a fluorophore linked by a short ssRNA sequence to a quencher, which emits fluorescence after the ssRNA sequence is cleaved, indicating the presence, and therefore the detection, of the target of interest. Because different Cas13 proteins exhibit different cleavage preferences depending on ssRNA sequences [51], the ssRNA reporter that can be cleaved by TccCas13a and HheCas13a effectors was investigated.

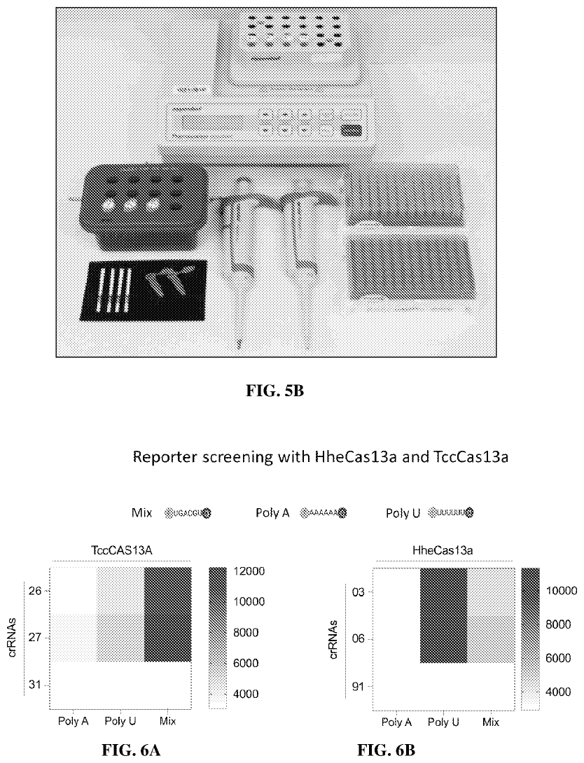

It was determined whether these proteins retain the non-specific trans degradation activities of ssRNA reporter molecules in the presence of ssRNA target at higher temperatures. Different Cas13a variants trigger collateral RNase activity at exposed uridine residues, so the trans-cleavage nucleotide preference of TccCas13a and HheCas13 at higher temperatures was tested. Therefore, 3 different ssRNA probes were screened. Each probe was conjugated to a 5′ fluorescent molecule (FAM) and a 3′ fluorescence quencher (FQ). 6-mers of poly(A) and poly(U) homopolymers and an additional 6-mer probe of mixed U, G, A, C nucleotides were used. Using two targeting crRNAs and one non-specific (NS) crRNA, it was observed that TccCas13a exhibited a trans-ssRNA cleavage preference against a mixed RNA sequence (not homopolymer ssRNA sequence) containing different nucleotides: UGACGU (FIG. 6A). HheCas13 exhibited a cleavage preference for a homo-uridine ssRNA substrate, consistent with previous observations (FIG. 6B) [55]. Moreover, the rate at which the trans-cleavage activity of ssRNA reporters reached saturation was determined using different target, ssRNA activator, concentrations. The data show that TccCas13 exhibited a higher sensitivity with only fM of the ssRNA activator resulting in detectable cleavage of the reporter.

Based on the observation that both HheCas13a and TccCas13a are derived from thermophilic host bacteria, it was determined whether both effectors exhibit cleavage activity at high temperatures. It was observed that both proteins were highly active at high temperature (60° C.), with TccCas13a showing stronger and faster activity at high temperature compared with its activity at 37° C. (FIGS. 7A-7B).

These results prompted the investigation of the activity of both Cas13 effectors at a broad range of elevated temperatures. HheCas13a maintained strong activity at temperatures up to 60° C. On the other hand, TccCas13a maintained a robust activity at temperatures as high as −70° C. (FIG. 7C). In addition, crRNAs with increased spacer lengths was tested using 24 and 28 nt long spacers. Subsequently, trans cleavage activity assays using a broad temperature range (37-72 C) was conducted. The data showed that TccCas13a loaded with crRNA2 is active over a wide range of temperature (37-70 C). HheCas13, however, showed, robust activity between 42-60 C. Upon exploring the crRNA spacer length requirements of the previously uncharacterized TccCas13a effector, it was observed that TccCas13a showed comparable activity using 24 or 28 nt long spacer sequences (FIG. 7D). These data provide compelling evidence on the thermostability and catalytic activities of TccCas13a and HheCas13a proteins and their usefulness in applications requiring cis and trans catalytic activities.

Use of Thermophilic Cas13s for SARS-CoV-2 Detection in One-Pot and Two-Pot Assays

To ensure sensitive detection, pre-amplifying the RNA target of interest is a necessary step [52]. RT-LAMP isothermal amplification was chosen because it possesses several advantages over other amplification methods, including high sensitivity, rapid turnaround time, simple operation, and low cost [56]. Primer sets well-established in previous reports were used to target and amplify conserved regions in the SARS-CoV-2 N gene, named here as STOPCovid (SC) primer sets [49, 57]. However, because Cas13s target RNA, these primers were modified by appending a T7 promoter sequence to the 5′ end of the first half of either the forward inner primer (FIP) or the backward inner primer (BIP). Therefore, during LAMP amplification, the T7 promoter sequence should get integrated into the amplified DNA products, providing a suitable template for the T7 RNA polymerase to transcribe the amplified LAMP product in vitro and generate RNA targets for Cas13 detection (Table 5).

Various reports have shown the utilization of T7 RNA polymerase for subsequent in vitro transcription of the amplified product and Cas13 based detection. However, coupling of the RT-LAMP amplification, T7-mediated transcription, and Cas13 detection in one pot at relatively high temperature suitable for RT-LAMP amplification is an unmet need. Therefore, the thermostable Hi-T7 RNA polymerase, which exhibits optimal performance at temperatures close to where both RT-LAMP and thermophilic Cas13 variants are active, was utilized to accomplish this goal.

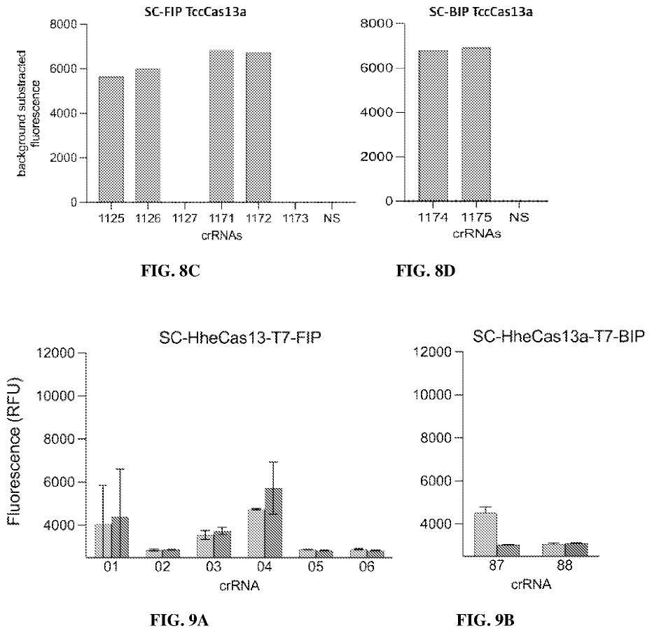

crRNAs targeting the highly conserved region in the SARS-CoV-2 N-gene were designed and tested with primer sets previously reported to be highly efficient and specific. Initial screening of these crRNAs and primer sets in two-pots settings, where RT-LAMP was performed first, and the amplified product was used in the second step for T7 RNA polymerase-mediated in-vitro transcription and Cas13-based detection, identified few crRNAs that were highly active (FIGS. 8A-8D).

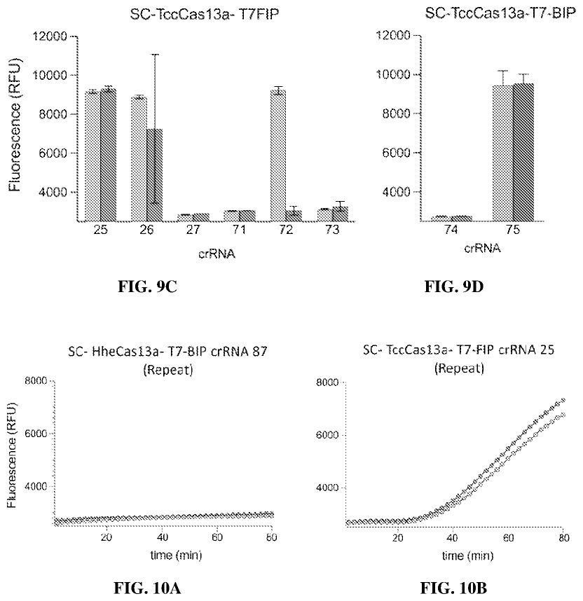

It was then investigated whether target detection in one-pot assay is feasible at a single temperature using all three enzymes. Therefore, all crRNAs and primer sets were rescreened in one pot settings. The screening assays identified a combination of crRNA and primer set that showed the most specific and efficient detection of the SARS-CoV-2 RNA in comparison to the other tested crRNAs and primers, namely crRNA #1172 when used with SC T7-FIP modified primer sets (FIGS. 9A-9D).

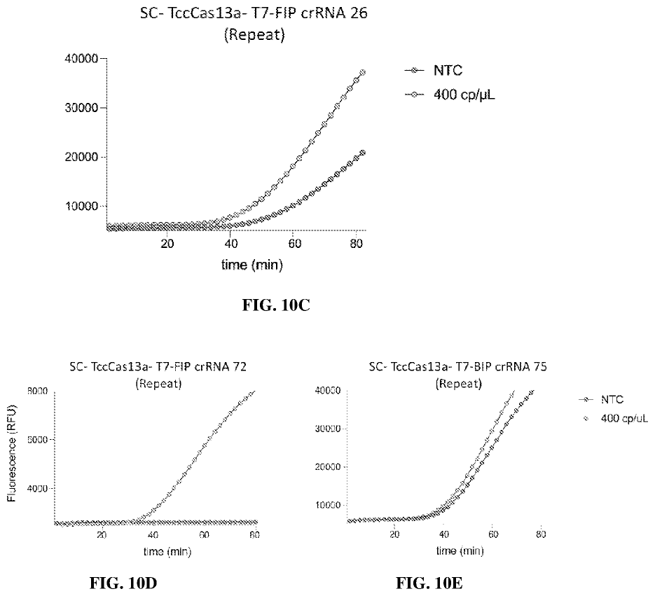

Consistently, this combination of primers and crRNA specifically and efficiently detected SARS-CoV-2 target in one-pot (FIGS. 10A-10E).

Optimization of the One-Pot Assay

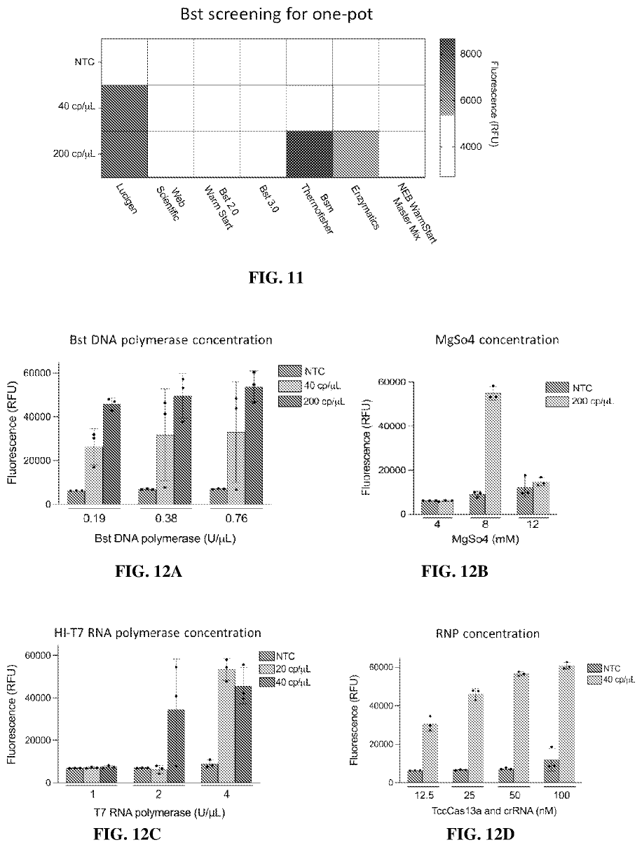

In order to maximize the efficiency of this system, the reaction chemistry was optimized in terms of the type of Bst DNA polymerase used, and the concentrations of Bst DNA polymerase, Hi-T7 RNA polymerase, Mg²⁺ and Cas13 RNP in the reaction (FIG. 11 and FIGS. 12A-12D). It was observed that the optimal sensitivity and efficiency of the one-pot detection assay can be achieved if the system is highly tuned with regards to the type and concentration of enzymes used at every step (FIG. 11 and FIGS. 12A-12D).

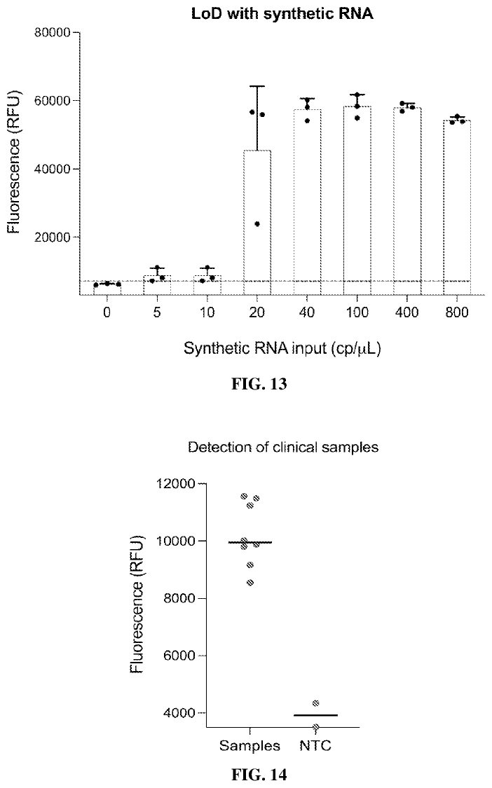

With the optimized reaction, the analytical limit of detection (LoD) of the Cas13-based one pot assay was evaluated using synthetic SARS-CoV-2 RNA as an input. The LoD of the one pot assay was estimated to be 20 cp/uL, an improved sensitivity relative to reported Cas13-based one pot assays for SARS-CoV-2 detection (FIG. 13) [58].

Validation of the Thermophilic Cas13-Based One-Pot Assay on Clinical Samples

Next, the assay was validated with total RNA extracted from SARS-CoV-2 patient swab samples. Oropharyngeal or nasopharyngeal swab samples were collected from suspected COVID-19 patients. After RNA extraction following the CDC EUA-approved protocol, the samples were confirmed positive for SARS-CoV-2 using RT-qPCR. The Cas13-based one-pot assay was first tested on 8 samples with Ct values of 14-27. Using the one-pot Cas13 detection assay, all samples were correctly identified (FIG. 14). These results indicate that this newly developed detection system can reliably detect SARS-CoV-2 in clinical samples.

Discussion

Initially, CRISPR/Cas diagnostic systems were designed as two-pot assays [49, 50, 59], where reverse transcription and isothermal reactions were performed separately. Following the amplification step, a fraction of the reaction mixture was transferred to another tube containing the Cas enzyme and reporter. This approach, however, dramatically increases the chances of carry-over contamination and complicates handling, rendering such modules unfeasible as real-life diagnostic kits. Numerous attempts were reported to address these shortcomings [60] including one-pot reaction systems using Cas12b [61] and amplification-free detection protocols [62, 63]. Amplification-free strategies based on LbuCas13a have the advantage of facile protocol and absence of cross-contamination. However, sensitivity of such approaches remains low, with samples having Ct values above 29 evading detection. Furthermore, all present amplification-free techniques rely on a sophisticated device that must distinguish between genuine signal and the background. An advantage of the thermophilic Cas13-based one-pot detection assay described herein is the straightforward assay workflow that can be performed without sophisticated equipment or trained personnel. The whole assay can be performed in less than 80 minutes with only minimal equipment or risk of cross-contamination.

Frequent, rapid and cost-effective testing without relying on centralized facilities is a key advancement in early screening during pandemic situations [64]. It is believed that the thermophilic Cas13-based one-pot detection assay described herein represents the first report of RT-LAMP coupled with a thermostable Cas13 ortholog for one-pot detection of SARS-CoV-2 that shows great promise in complimenting existing methods.

### Example 3: Development of a Miniature CRISPR-Cas13 System that Facilitates SARS-CoV-2 Detection

CRISPR/Cas systems possess great potential for various applications, so there are ongoing efforts to search for, identify, and characterize new Cas effectors to increase utility and develop new tools for in vivo and in vitro applications [93, 94]. Recently, computational approaches for metagenomic mining resulted in the discovery of previously unknown CRISPR/Cas systems, including class II/type VI Cas proteins that exclusively target ssRNA substrates [95-98]. Besides repurposing these RNA targeting CRISPR/Cas13s for in vivo applications [99-103], Cas13s can be used in diagnostics that exhibit unprecedented sensitivity, specificity, and speed [85, 87-89, 92]. Different Cas13 variants have been used for nucleic acid detection. For example, Cas13a [86, 104], Cas13b [105], and Cas13d [106] exhibit collateral cleavage activities and work for nucleic acid detection. This Example expands the existing Cas13-based toolbox for diagnostic applications by identifying and characterizing novel CRISPR/Cas13 effectors. In this Example, an mCas13 variant was identified, characterized, and its utility for SARS-CoV-2 detection demonstrated. This work illustrates the untapped potential of mCas13 enzymes in diagnostics and other in vivo RNA applications.

Materials and Methods

Computational Identification of CRISPR/mCas13

Protein sequences of miniature Cas13s in a recent report [107] were kindly provided by Dr. Hui Yang (Chinese Academy of Sciences, Beijing, China). These protein sequences were used as queries in the Basic Local Alignment Search Tool (BLAST) against the NCBI non-redundant (nr) protein database (before January 2021) using default settings. Only subject sequences with query coverage (Query cover) above 90% were considered. Protein sequences of the miniature Cas13f variants did not identify any subject sequences with query coverage above 90%. However, when using protein sequences of the miniature Cas13e variants, especially Cas13e.1 (accession #RKY08123), mCas13 (accession #HFH51004) showed up as the only subject sequence with query coverage above 90% (96%). Protein sequence alignment of mCas13 and the miniature Cas13e and Cas13f variants was performed using ClustalW [118] in MEGAX with default settings, and the alignment was visualized using ESPript [119]. The RxxxxH HEPN motif was subsequently identified in the mCas13 protein sequence on the basis of this alignment and was further confirmed by manually searching for this motif using SnapGene. CRISPRCasFinder [120] was performed on the genomic DNA sequence (GenBank #DSVK01000191.1) to identify the associated CRISPR array. CRISPRDetect [121] was then used to predict the orientation of the direct repeat in the mCas13 CRISPR array.

Cas13 Protein Expression and Purification

To produce the expression plasmid for Cas13 expression and purification, the E. coli codon-optimized Cas13 coding sequence was synthesized (GenScript) de novo and subcloned in frame with His and SUMO tags on the N-terminus into the His6-TwinStrep-SUMO bacterial expression vector (Addgene #115267) using BamHI and NotI (Table 12). Purification of mCas13 protein was performed following the protocol of Kellner et al. (2019) with a few modifications. Briefly, the mCas13 expression vector was transformed into BL21 E. coli cells. Starter cultures were prepared by growing single colonies in LB broth supplemented with 100 μg/mL ampicillin for 12 h at 37° C. Next, 20 mL of starter culture was used to inoculate 2 L of Terrific Broth medium (TB) (IBI scientific) supplemented with 100 μg/mL ampicillin for growth at 37° C. until an OD₆₀₀ of 0.5. Cells were incubated on ice for 30 mins, expression was induced with 0.5 mM IPTG, and cultures were then transferred to 16° C. for overnight expression. Cells were harvested by centrifugation for 20 min at 4° C. at 4000 rpm. Cell pellets were resuspended in lysis buffer (50 mM Tris-Cl pH 7.5, 500 mM NaCl, 5% glycerol, 1 mM DTT, EDTA-free protease inhibitor (Roche)) and supplemented with 1 mg/mL lysozyme (L6876, Sigma). Cells were lysed by sonication and clarified by centrifugation at 11,000 rpm for 50 min. The soluble 6×His-SUMO-mCas13 in cleared lysate was then purified with an affinity chromatography column (HiTrap Q HP, 5 mL GE Healthcare) (AKTA PURE, GE Healthcare) followed by concurrent removal of the 6×His-SUMO tag by SUMO protease and overnight dialysis in dialysis buffer. Cleaved protein was concentrated to 1.5 mL by Amicon Ultra-15 Centrifugal Filter Units (50 kDa NMWL, UFC905024, Millipore) and further purified via size-exclusion chromatography on a 5200 column (GE Healthcare) in gel filtration buffer (50 mM Tris-HCl, 600 mM NaCl, 10% glycerol, 1 mM DTT, pH 7.5). The protein-containing fractions resulting from the gel filtration were pooled, snap frozen, and stored at −80° C.

Nucleic Acid Preparation

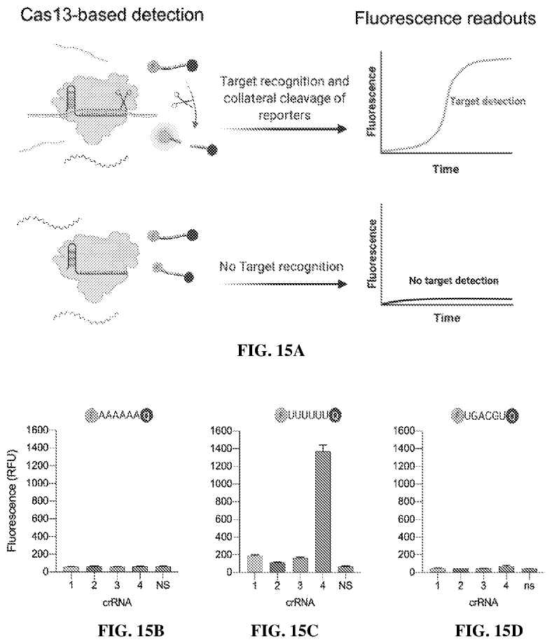

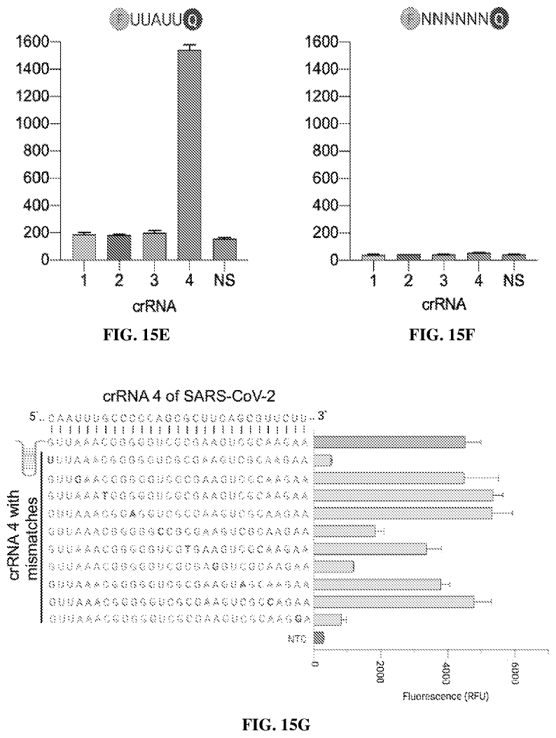

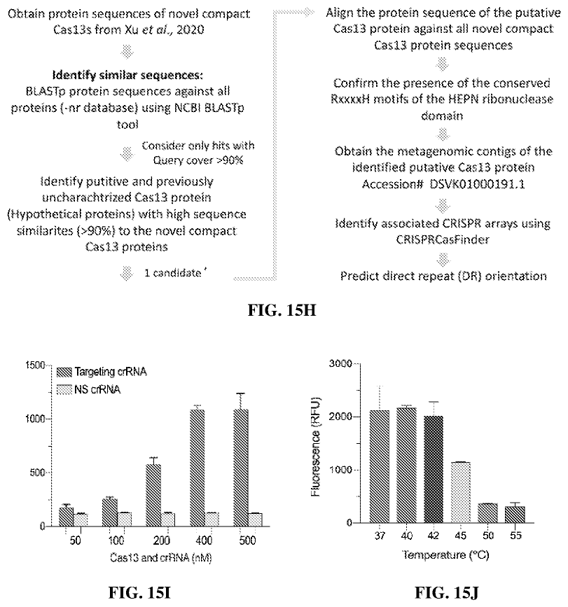

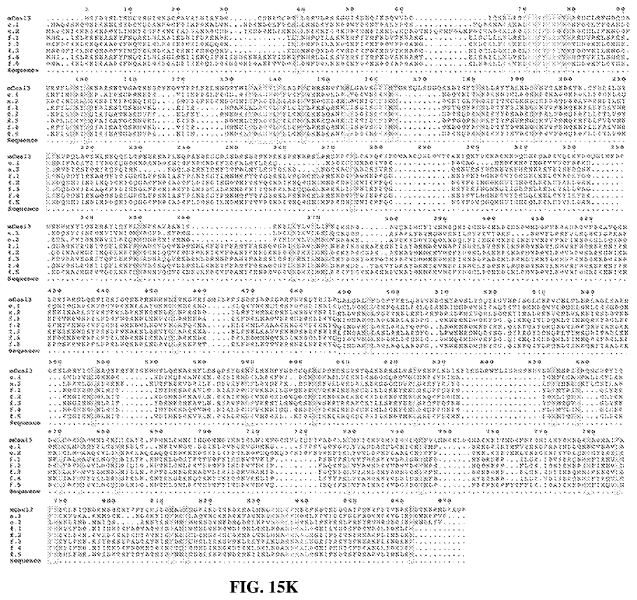

A short region of the SARS-CoV-2 N gene sequence was used as a synthetic target in the preliminary mCas13 characterization and optimization experiments to screen crRNAs and collateral reporters and establish mCas13-based detection (FIG. 15). The N gene target RNA sequences were prepared by in vitro transcription of PCR amplicons containing the T7 promoter sequence using the 2019-nCoV_N_Positive Control plasmid as a PCR template (10006625, IDT). Purified PCR amplicons (QIAquick PCR Purification Kit, QIAGEN) were transcribed in vitro using HiScribe T7 Quick High Yield RNA Synthesis Kit (E2050, NEB). The transcripts were purified using Direct-zol RNA Miniprep Kits (R2050, Zymo Research) following the manufacturer's instructions, and the purified RNA was stored at −80° C.

mCas13 crRNAs were designed to target the N gene sequence of the SARS-CoV-2 genome. For crRNA preparation, templates for in vitro transcription were generated using single-stranded DNA oligos containing a T7 promoter, scaffold, and spacer in reverse complement orientation (IDT), and were then annealed to T7 forward primer in Taq DNA polymerase buffer (Invitrogen). The annealed oligos were then used as templates for in vitro transcription as described above.

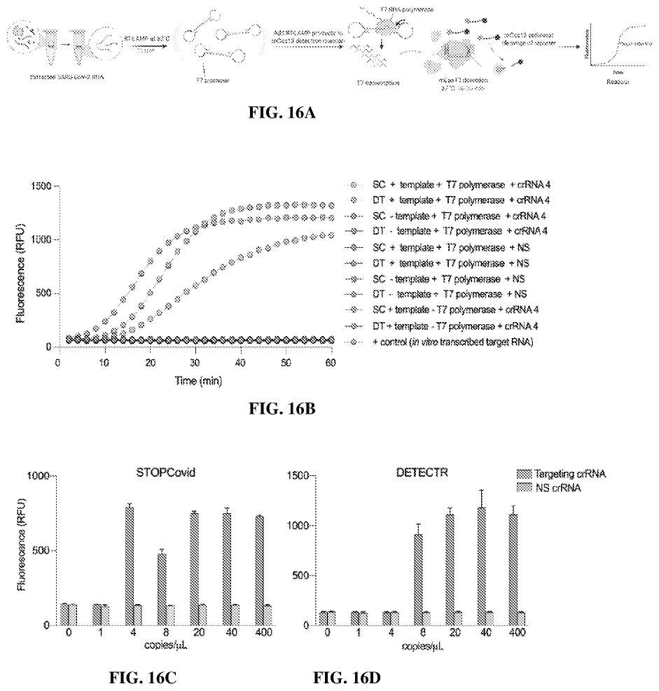

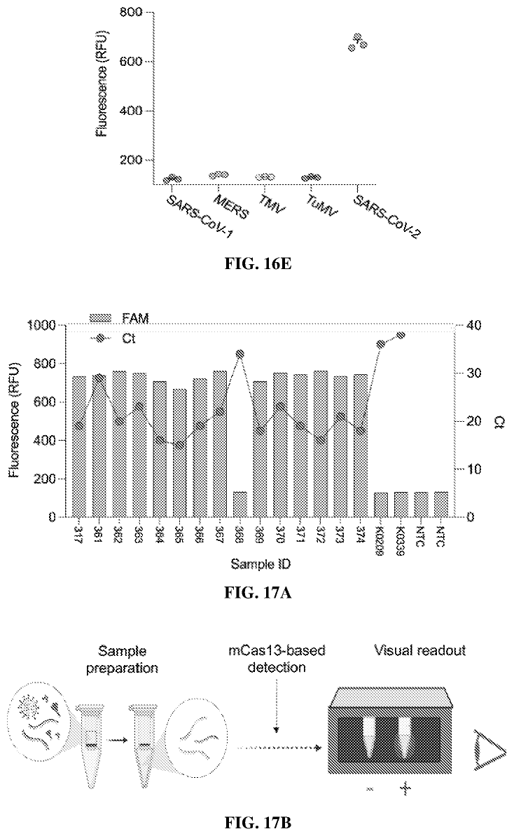

To establish RT-LAMP coupled with T7-mCas13-based detection and LoD range, control synthetic SARS-CoV-2 viral genomic sequences used in FIG. 16 were ordered as synthetic RNA from Twist Bioscience, and were diluted to 10,000 RNA copies/μL and used at indicated concentrations to create simulated clinical samples.

For RT-LAMP amplification (described below), previously published LAMP primers designed to amplify the SARS-CoV-2 N gene (Joung et al., 2020 [81], Broughton et al., 2020 [82]) were used, with modifications. The FIP or BIP primers were each designed with the appended T7 promoter sequence at the 5′ end of the first half of the primer. Such modification allows the modified primer to integrate the T7 promoter sequence in the LAMP-amplified product for subsequent T7-mediated in vitro transcription. All oligo sequences and substrates are listed in Tables 5, 6, and 11.

Screening of crRNAs and Reporters and Establishing mCas13 Collateral Detection

Activity and collateral assays of Cas13 were performed in 1×cleavage buffer (20 mM HEPES-Na pH 6.8, 50 mM NaCl, 5 mM MgCl₂, 1 mM DTT) in a 20-4 final reaction volume. Cas13 and crRNAs RNPs were first assembled by mixing 500 nM purified Cas13 with 500 nM crRNA (unless otherwise indicated) in 1×cleavage buffer and 20 units RNaseOUT (Invitrogen), followed by incubation at 37° C. for 15 minutes. Next, the assembled RNP was combined on ice with 2 μL of 500 ng/μL in vitro-transcribed target RNA and 250 nM RNA reporter, and reactions incubated for 1 hr at 37° C. (unless otherwise indicated). Real time or end-point fluorescence measurements were collected on a microplate reader M1000 PRO (TECAN) at 2-min intervals (for real time measurements) using 384-well, black/optically clear flat-bottomed plate (Thermofisher).

RT-LAMP Reactions

Reverse transcription and isothermal amplification of target nucleic acids were performed using final concentrations of 1.6μM FIP/BIP primers (with the T7 promoter sequence fused to either the FIP or BIP primer), 0.2 μM F3/B3 primers, and 0.4 μM LF/LB primers, 1×Isothermal Amplification Buffer (20 mM Tris-HCl, 50 mM KCl, 10 mM (NH₄)₂SO₄, 2 mM MgSO4, 0.1% Tween 20, pH 8.8) (B0537, NEB), 1.4 mM dNTPs, 8 units of Bst2.0 WarmStart DNA Polymerase (M0538, NEB), 7.5 units of WarmStart RTx Reverse Transcriptase (M0380, NEB) and 6 mM MgSO4 (B1003, NEB) in 25-4, reactions containing variable concentrations of SARS-CoV-2 control standards, or 4 μL of isolated RNA from clinical samples. LAMP reactions were performed at 62° C. for 35 minutes in a PCR (C1000 touch thermal cycler, BioRad) machine.

One-Step T7 Transcription and mCas13 Detection

The two reactions, T7-mediated in vitro transcription and mCas13-based detection of the amplified and in vitro-transcribed target RNA, were carried out in the same tube. Briefly, 2 μL of the RT-LAMP reaction product was combined with 1×cleavage buffer (described above), 500 nM mCas13/crRNA assembled RNPs, 25 units T7 RNA polymerase (M0251, NEB), 1 mM NTPs, and 250 nM RNA reporter in 20-μL reactions. The reactions were incubated at 37° C. for 20-30 minutes.

Visual Cas13-Based Detection

For simple visualization of mCas13-based detection, RNA reporters labeled with the HEX fluorophore were used instead of the FAM fluorophore (Table 7). Collateral cleavage of HEX reporters results in a bright signal that can be easily visualized upon excitation with LED light (Ali et al., 2020). Cas13-based reactions were carried out as described above, with modifications. For each reaction, 1 μM of HEX reporter (unless otherwise indicated) was used in 20-μL T7-mCas13 detection reactions. Reactions were incubated at 37° C. for 30 minutes. Reaction tubes were then transferred into the P51 Molecular Fluorescence Viewer (miniPCR) and photos were taken using a smartphone with default settings.

Clinical Sample Collection and RNA Extraction

Oropharyngeal and nasopharyngeal swabs were collected from suspected COVID-19 patients by physicians in Ministry of Health hospitals in Saudi Arabia and placed in 2-mL screw-capped cryotubes containing 1 mL of TRIZOL for inactivation and transport. Each sample tube was sprayed with 70% ethanol, enveloped with absorbent tissues, then placed and sealed in an individual labeled biohazard bag. The bags were then placed in leak-proof boxes and sprayed with 70% ethanol before placement in a dry ice container for transfer to King Abdullah University of Science and Technology (KAUST). Total RNA was extracted from the samples following instructions as described in the CDC EUA-approved protocol and using the Direct-zol kit (Direct-zol RNA Miniprep, Zymo Research; catalog #R2070) following the manufacturer's instructions.

Real-Time Reverse Transcription PCR (RT-PCR) for Detecting Positive SARS-CoV2 RNA Samples.

RT-PCR was conducted on extracted RNA samples using the oligonucleotide primer/probe (Integrated DNA Technologies, catalog #10,006,606) and Superscript III one-step RT-PCR system with Platinum Taq Polymerase (catalog #12574-026) following the manufacturer's protocol.

Nucleotide and Amino Acid Sequences Used

TABLE 11
crRNA sequences used in this study.

| Name | Sequence | Note |
|---|---|---|
| Sense-mCas13-T1 | GCTGGAGCAGCCCTCGATTTGCAGGGTAATCACA | crRNAs for |
| | GCACAAGGAACTGATTACAAACATTGGCCGCACC | SARS-CoV-2 |
| | CTATAGTGAGTCGTATTAATTTC (SEQ ID NO: 72) | N gene |
| Sense-mCas13-T2 | GCTGGAGCAGCCCTCGATTTGCAGGGTAATCACA | Target 1 |
| | GCACTGATTACAAACATTGGCCGCAAATTGCACC | |
| | CTATAGTGAGTCGTATTAATTTC (SEQ ID NO: 73) | |
| Sense-mCas13-T3 | GCTGGAGCAGCCCTCGATTTGCAGGGTAATCACA | |
| | GCCGTTCTTCGGAATGTCGCGCATTGGCATGGCC | |
| | CTATAGTGAGTCGTATTAATTTC (SEQ ID NO: 74) | |
| Sense-mCas13-T4 | GCTGGAGCAGCCCTCGATTTGCAGGGTAATCACA | |
| | GCCAATTTGCCCCCAGCGCTTCAGCGTTCTTCCCT | |
| | ATAGTGAGTCGTATTAATTTC (SEQ ID NO: 75) | |
| Sense-mCas13-N | GCTGGAGCAGCCCTCGATTTGCAGGGTAATCACA | crRNAs for |
| target 2-1 | GCAAGATCACATTGGCACCCGCAATCCTGCTACC | SARS-CoV-2 |
| | CTATAGTGAGTCGTATTAATTTC (SEQ ID NO: 76) | N gene |
| Sense-mCas13-N | GCTGGAGCAGCCCTCGATTTGCAGGGTAATCACA | Target 2 |
| target 2-2 | GCACAATGCTGCAATCGTGCTACAACTTCCTCCCT | |
| | ATAGTGAGTCGTATTAATTTC (SEQ ID NO: 77) | |
| Sense-mCas13-N | GCTGGAGCAGCCCTCGATTTGCAGGGTAATCACA | |
| target 2-3 | GCCGTTCCTCATCACGTAGTCGCAACAGTTCACC | |
| | CTATAGTGAGTCGTATTAATTTC (SEQ ID NO: 78) | |
| Sense-mCas13-N | GCTGGAGCAGCCCTCGATTTGCAGGGTAATCACA | |
| target 2-4 | GCACAGTTCAAGAAATTCAACTCCAGGCAGCACC | |
| | CTATAGTGAGTCGTATTAATTTC (SEQ ID NO: 79) | |
| Sense-mCas13-N | GCTGGAGCAGCCCTCGATTTGCAGGGTAATCACA | |
| target 2-5 | GCGATGCTGCTCTTGCTTTGCTGCTGCTTGACCCT | |
| | ATAGTGAGTCGTATTAATTTC (SEQ ID NO: 80) | |
| Sense-mCas13-N | GCTGGAGCAGCCCTCGATTTGCAGGGTAATCACA | |
| target 2-6 | GCAGGCCAAACTGTCACTAAGAAATCTGCTGCCC | |
| | TATAGTGAGTCGTATTAATTTC (SEQ ID NO: 81) | |
| Sense-mCas13-NS | GCTGGAGCAGCCCTCGATTTGCAGGGTAATCACA | Non-specific |
| | GCTTATGCCTCTTCCGACCATCAAGCATTTTACCC | targeting |
| | TATAGTGAGTCGTATTAATTTC (SEQ ID NO: 82) | |
| Sense-mCas13-4- | GCTGGAGCAGCCCTCGATTTGCAGGGTAATCACA | |
| Mis-1 | GCAAATTTGCCCCCAGCGCTTCAGCGTTCTTCCCT | |
| | ATAGTGAGTCGTATTAATTTC (SEQ ID NO: 83) | |
| Sense-mCas13-4- | GCTGGAGCAGCCCTCGATTTGCAGGGTAATCACA | |
| Mis-2 | GCCAACTTGCCCCCAGCGCTTCAGCGTTCTTCCCT | |
| | ATAGTGAGTCGTATTAATTTC (SEQ ID NO: 84) | |
| Sense-mCas13-4- | GCTGGAGCAGCCCTCGATTTGCAGGGTAATCACA | |
| Mis-3 | GCCAATTTACCCCCAGCGCTTCAGCGTTCTTCCCT | |
| | ATAGTGAGTCGTATTAATTTC (SEQ ID NO: 85) | |
| Sense-mCas13-4- | GCTGGAGCAGCCCTCGATTTGCAGGGTAATCACA | |
| Mis-4 | GCCAATTTGCCTCCAGCGCTTCAGCGTTCTTCCCT | |
| | ATAGTGAGTCGTATTAATTTC (SEQ ID NO: 86) | |
| Sense-mCas13-4- | GCTGGAGCAGCCCTCGATTTGCAGGGTAATCACA | |
| Mis-5 | GCCAATTTGCCCCCGGCGCTTCAGCGTTCTTCCCT | |
| | ATAGTGAGTCGTATTAATTTC (SEQ ID NO: 87) | |
| Sense-mCas13-4- | GCTGGAGCAGCCCTCGATTTGCAGGGTAATCACA | |
| Mis-6 | GCCAATTTGCCCCCAGCACTTCAGCGTTCTTCCCT | |
| | ATAGTGAGTCGTATTAATTTC (SEQ ID NO: 88) | |
| Sense-mCas13-4- | GCTGGAGCAGCCCTCGATTTGCAGGGTAATCACA | |
| Mis-7 | GCCAATTTGCCCCCAGCGCTCCAGCGTTCTTCCCT | |
| | ATAGTGAGTCGTATTAATTTC (SEQ ID NO: 89) | |
| Sense-mCas13-4- | GCTGGAGCAGCCCTCGATTTGCAGGGTAATCACA | |
| Mis-8 | GCCAATTTGCCCCCAGCGCTTCATCGTTCTTCCCT | |
| | ATAGTGAGTCGTATTAATTTC (SEQ ID NO: 90) | |
| Sense-mCas13-4- | GCTGGAGCAGCCCTCGATTTGCAGGGTAATCACA | |
| Mis-9 | GCCAATTTGCCCCCAGCGCTTCAGCGGTCTTCCCT | |
| | ATAGTGAGTCGTATTAATTTC (SEQ ID NO: 91) | |
| Sense-mCas13-4- | GCTGGAGCAGCCCTCGATTTGCAGGGTAATCACA | |
| Mis-10 | GCCAATTTGCCCCCAGCGCTTCAGCGTTCCTCCCT | |
| | ATAGTGAGTCGTATTAATTTC (SEQ ID NO: 92) | |

TABLE 12
mCas13 protein sequence and tag sequences for protein
purification.

| Name | Protein sequence |
|---|---|
| mCas13 | MGSSHHHHHHSSGLVPRGSHMASWSHPQFEKGGGSGGGSGGSAW |
| | SHPQFEKMSDSEVNQEAKPEVKPEVKPETHINLKVSDGSSEIFFKIKK |
| | TTPLRRLMEAFAKRQGKEMDSLRFLYDGIRIQADQTPEDLDMEDND |
| | IIEAHREQIGGSMGIDYSLTSDCYRGINKSCFAVALNIAYDNCDHKG |
| | CRTLLSEVLRSKGGISDEQIKSQVVDGIQKRLKDIRNYFSHYYHAED |
| | CLRFGDQDAVKVFLEEIYKNAESKTVGATKESDYKGVVPPLFELHN |
| | GTYMITAAGVIFLASFFCHRSNVYRMLGAVKGFKHTGKEQLSDGQ |
| | KRDYGFTRRLLAYYALRDSYSVGAEDKTRCFREILSYLSRVPQLAV |
| | DWLNEQQLLTPEEKEAFLNQPAEDEGGDISDSSSSDKNKKSKEKRR |
| | SLRRDEKFILFAIQFIEGWAAEQGLDVTFARYQKTVEKAENKNQDG |
| | KQARAVQLKYRNQGLNPDFNNEWMYYIQNEHAIIQIKLNNKKAVA |
| | ARISENELKYLVLLIFEEKGNDAVQKLNCYIYSMSQKIEGEWKHRPE |
| | DERWMPSFTKRADRTVTPEAVQSRLSYIRKQLQETIEKIGQEEPRNN |
| | KWLIYKGKKISMILKFISDSIRDIQRRPNVKQYHILRDALQRLDFDGF |
| | YKELQNYVNDGRIAVSLYDQIKGVNDISGLCKKVCELTLERLAGLE |
| | AKNGSELRRYIGLEAQEKHPKYGEWNTLQEKAKRFLESQFSIGKNF |
| | LRKMFYGDCCQKRCFDEEKGYNTQAKERKSLYSIVKEKLKDIKPIH |
| | DDRWYLIDRNPKNYDNKHSRIIRQMCNTYIQDVLCMKMAMWHYE |
| | KLISATEFRNKLEWNCIGQGNMGYERYSLWYKTGCGVVIQFTPADF |
| | LRLDIIEKPAMIENICQCFVLGNKKLNSGAEKKITWDKFNKDGIAKY |
| | RKRQAEAVRAIFAFEEGLKIQEDKWSHERYFPFCNILDEAVKQGKIK |
| | DTGKDKEALNRGRNDFFHEEFKSTEDQQAIFQKYFPIVERKDDTKK |
| | RRDKKQK* (SEQ ID NO: 93) |

6x His affinity tag: residues 5-10; Thrombin site: residues 14-19; Strep-tag II: residues 24-31 and 44-51; SUMO: residues 52-148; mCas13 protein: residues 151-1023.

Results

Identification, Design, Construction, and Expression of a Novel Miniature Cas13 System

Xu et al. [107] describes compact Cas13 effectors that are classified as CRISPR/Cas type VI-E and VI-F. These compact Cas13s are used for in vivo applications, including endogenous RNA interference, RNA editing, and as an anti-coronavirus approach that targets and combats SARS-CoV-2 and other influenza viruses in vivo [107].

Some of the compact Cas13 protein sequences from Xu et al. were used as queries in the Basic Local Alignment Search Tool (BLAST) to find potentially uncharacterized mCas13 proteins. The alignment identified various proteins, including a few uncharacterized putative Cas13 sequences with two predicted RxxxxH motifs of the conserved Cas13 Higher Eukaryotes and Prokaryotes Nucleotide-binding (HEPN) ribonuclease domains [97, 108]. One small (837 amino acids) candidate showed high similarity (>95% query coverage) to the most efficient Cas13 identified in Xu et al., namely Cas13e.1 [107]. Further analysis of the metagenomic contigs showed that the putative mCas13 protein has an associated CRISPR array in its immediate vicinity. In silico analysis of the associated CRISPR array predicted that the mCas13-associated crRNAs share high similarity with the length and architecture of the compact Cas13e.1 crRNA (30-nt long spacer sequence at the 5′ end of the crRNA followed by a 36-nt long direct repeat (DR) sequence at the 3′ end), and to previously reported crRNAs of the Cas13b family [100]. A schematic illustration of the computational strategy is shown in FIG. 15H. Furthermore, multiple sequence alignment analysis showed high similarity and the two predicted HEPN domains conserved among the putative mCas13 protein, Cas13e.1, and the other compact proteins, Cas13e and Cas13f (FIG. 15K). Based on these predictions, it was hypothesized that the putative mCas13 is functionally active. Next, the corresponding gene sequence was codon-optimized and synthesized and a bacterial expression plasmid for heterologous expression in BL21 Escherichia coli containing the mCas13 sequence was designed and constructed. Subsequently, the protein was produced, and its activity tested in vitro for diagnostic applications.

Characterization of CRISPR-mCas13 Cis and Trans Catalytic Activities

When Cas13/crRNA ribonucleoproteins (RNP) recognize and cleave their target sequence, they also exhibit non-specific, collateral cleavage activity that degrades ssRNAs nearby [108, 109]. Such collateral activity can be used in nucleic acid detection applications, where a ssRNA probe (reporter) molecule provided in the Cas13 reaction is cleaved by target-dependent Cas13 collateral activity [86]. The ssRNA reporter can contain a fluorophore linked by a short ssRNA sequence to a quencher, which emits fluorescence after the ssRNA sequence is cleaved, indicating the presence, and therefore the detection, of the target sequence (FIG. 15A).

To test the mCas13's cis and in-trans activities in vitro and to determine the most effective crRNAs to use in the mCas13 SARS-CoV based detection assays, 10 different crRNAs targeting two different regions in the SARS-CoV-2 nucleocapsid gene (N) were designed and screened. The in vitro cleavage activity of mCas13 was first evaluated with 4 different crRNAs targeting single-stranded RNA substrates harboring target sequences complementary to the crRNA spacers. mCas13 exhibited different cleavage efficiencies with different crRNAs, with crRNA 4 mediating the highest efficiency relative to other crRNAs and controls.

Because different Cas13 proteins can exhibit different cleavage preferences depending on ssRNA sequences [88], efforts were directed to identifying the best ssRNA reporter for the mCas13 SARS-CoV-2 based detection module. Therefore, 5 different ssRNA probes, each conjugated to a 5′ fluorescent molecule (FAM) and a 3′ fluorescence quencher (FQ) were screened. mCas13 was incubated with each of the four targeting crRNAs and non-specific (NS) crRNA and reporters in the presence of the synthetic (N gene) ssRNA target. The screening consistently identified crRNA 4 with a significantly higher fluorescence signal relative to the NS crRNA control, indicating the cleavage preference of mCas13 for poly(U) reporter sequences (FIGS. 15B-15F). Using the poly (U) reporter molecule to screen six more crRNAs targeting different N gene regions indicated that different crRNAs exhibited overall different signal levels.

To determine the optimal concentration of mCas13 and crRNA for maximal detection signal, the reaction was performed with titrated mCas13 and crRNA concentrations. It was observed that the optimal concentration of Cas13/crRNA RNP for a true positive signal with no significant signal in NS crRNA control was 500 nM (FIG. 15I). In addition, to determine the optimal temperature for mCas13 catalytic activity, different cleavage temperature conditions were tested ranging from 37° C. to 55° C. The highest activity was observed at 37° C., which is similar to the optimal temperature of other known Cas13 enzymes used for nucleic acid detection (FIG. 15J).

Using optimized conditions and the most effective crRNA 4, the effect of single mismatches between crRNA and target RNA on mCas13 RNA detection activity was investigated. 10 crRNAs (based on crRNA 4) were designed to contain a single nucleotide mismatch at different sites, with one mismatch for every 3 nucleotides on the spacer sequence. A low mismatch tolerance should cause a much lower detection signal than the positive control (which is a perfect match). This analysis revealed different mismatch tolerances for different regions of the spacer sequence, where mismatches at the extreme 5′ or 3′ ends of the crRNA were not well tolerated. In contrast, mismatches at other regions were tolerated better (FIG. 15G). Altogether, these data indicated that the identified mCas13 is catalytically active with robust trans-cleavage activity, and thus is suitable for developing nucleic acid detection platforms.

RT-LAMP Coupled with CRISPR-mCas13 for SARS-CoV-2 Detection

To ensure sensitive detection, pre-amplifying the RNA target of interest is necessary [86]. RT-LAMP isothermal amplification was chosen because it possesses several advantages over other amplification methods, including high sensitivity, rapid turnaround time, simple operation, and low cost [110]. To initiate the RT-LAMP reaction, primer sets well-established in previous reports to target and amplify conserved regions in the SARS-CoV-2 N gene were used (named here as STOPCovid [81] and DETECTR [82] primer sets). Because mCas13 targets RNA, these primers were modified by appending a T7 promoter sequence to the 5′ end of the first half of either the forward inner primer (FIP) or the backward inner primer (BIP). During LAMP amplification, the T7 promoter sequence integrates into the amplified DNA products, providing a suitable template for the T7 RNA polymerase to transcribe the amplified LAMP product in vitro and generate RNA targets for mCas13 detection (FIG. 16A). The performance of these modified primers was tested using a synthetic SARS-CoV-2 viral genome at 500 copies/μL. Gel electrophoresis indicated that these modified primers successfully amplified the target RNA with no observed amplification in the no-template control (NTC) (not shown). In addition, no substantial differences were found between the amplifications using primer sets with T7-containing FIP primer (T7-FIP) or T7-containing BIP primer (T7-BIP). Therefore, T7-FIP primers were chosen to establish the RT-LAMP mCas13 detection platform.

Since mCas13 performs optimally at 37° C., a temperature also optimal for T7 RNA polymerase, the T7-mediated transcription of the RT-LAMP product was coupled with the Cas13-based detection of the transcribed RNA in a single tube. Therefore, after RT-LAMP pre-amplification of SARS-CoV-2 synthetic RNA using STOPCovid or DETECTR T7-FIP modified primers, the RT-LAMP products were added to the T7 transcription and mCas13 detection reaction. Real-time measurement of the T7-coupled mCas13-based detection indicated robust detection of RT-LAMP product only when using targeting crRNA (crRNA 4) and T7 RNA polymerase, confirming that the amplification of the synthetic SARS-CoV-2 genome was specific and that T7 promoters were successfully integrated into the amplified products (FIG. 16B). These results show the successful development of an mCas13-based two-pot SARS-CoV-2 detection platform.

Next, the limit of detection (LoD) of the two-pot SARS-CoV-2 detection assay was determined. When establishing the two-pot assay, it was verified that both STOPCovid and DETECTR primer sets could be used to effectively detect SARS-CoV-2 RNA (FIGS. 16C-16D). Therefore, the performance and the LoD of both STOPCovid and DETECTR primer sets were assayed to determine the primer set that is most suitable for the mCas13 SARS-CoV-2 detection platform. Serial dilutions of the synthetic SARS-CoV-2 viral genome were used as an input for the pre-amplification RT-LAMP reaction. It was observed that both primer sets allowed sensitive detection of the synthetic RNA, but the STOPCovid primers reproducibly detected as few as 4 copies/μL viral RNA, compared with 8 copies/μL for DETECTR primers (FIGS. 16C-16D). Although extending the RT-LAMP amplification time could enhance its sensitivity, the RT-LAMP pre-amplification step was limited to 35 mins and the mCas13 detection reaction was limited to 20-30 mins, resulting in a total detection time of ≈1 hour or less. Due to its outstanding LoD, the STOPCovid primer set was chosen for the mCas13 detection platform.

The RT-LAMP Coupled CRISPR-mCas13 Detection Assay is Specific for SARS-CoV-2

To test the assay's specificity and ensure no cross-reactivity with other common viruses, this system was challenged with other SARS-CoV related or non-related viruses, including SARS-CoV-1, MERS, TMV, and TuMV, together with SARS-CoV-2. All tested viruses (other than SARS-CoV-2) showed only near-background signals, indicating that the developed assay was highly specific (FIG. 16E). Collectively, these results indicated that the developed mCas13-based detection platform exhibits reliable, highly sensitive, and highly specific detection of SARS-CoV-2 with a turnaround time of 1 hour from extracted RNA to results.

Validation of RT-LAMP Coupled with CRISPR-mCas13 SARS-CoV-2 Detection Platform in a Clinical Setting

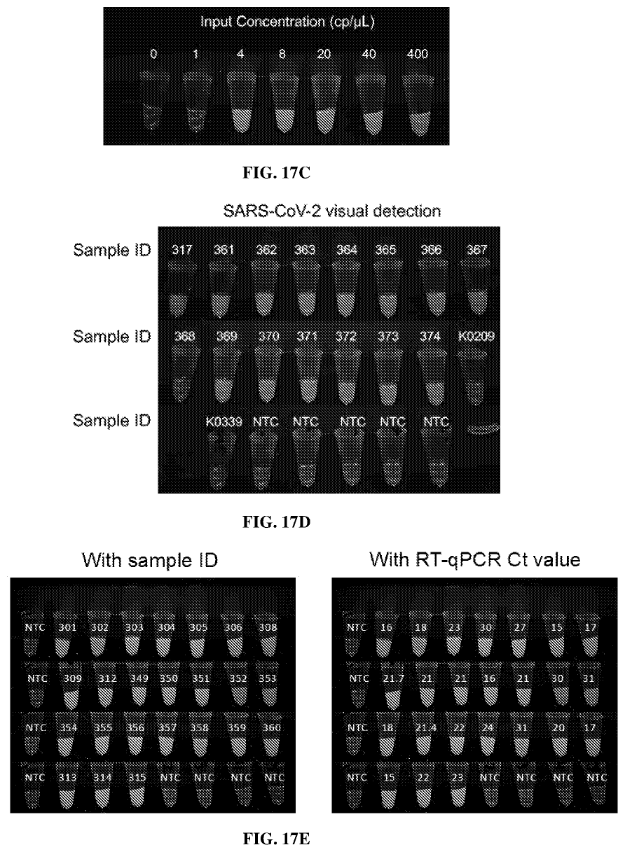

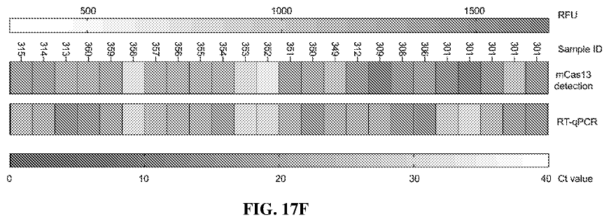

Next, the assay was validated with total RNA extracted from SARS-CoV-2 patient swab samples. Oropharyngeal or nasopharyngeal swab samples were collected from suspected COVID-19 patients. After RNA extraction following the CDC EUA-approved protocol, the samples were confirmed positive for SARS-CoV-2 using RT-qPCR. The assay was first tested with 17 samples with Ct values of 15-39. Using the mCas13-based detection assay, all samples with Ct values of less than 34 were correctly identified within one hour (FIG. 17A). These results indicate that the system can reliably detected SARS-CoV-2 in samples with Ct values up to 34.

To facilitate large-scale screening during the SARS-CoV-2 pandemic, performing diagnostic assays at POC or outside of laboratory settings is critical. Therefore, it was determined whether the assay could be coupled with a portable device to permit a simple readout suitable for POC and routine diagnostics. A hand-held, inexpensive fluorescence visualizer (P51 Molecular Fluorescence Viewer) was adapted to easily visualize and interpret the results. This portable device illuminates reaction tubes with blue light, and fluorescent reactions are visible through a film used as an optical filter. Using this device, fluorescence is readily visible to the human eye without the need for sophisticated instruments like qPCR machines or plate readers (FIG. 17B). Using a modified RNA reporter molecule conjugated to a 5′ HEX fluorescent molecule instead of FAM, it was observed that mCas13 collateral cleavage of this reporter produced a bright signal visible with the P51 fluorescence visualizer. Investigation of the HEX reporter concentration needed for definitive visual detection of true positive samples indicated that 1 μM of HEX RNA reporters produced a clear signal with no substantial background in negative controls.

To ensure that the sensitivity of the assay was preserved with the change in fluorescent molecule, the LoD assay was repeated using the STOPCovid primer set. The same results as obtained previously with the machine-based readout in FIG. 16C were obtained, demonstrating that the 5′ HEX modification did not affect assay performance (FIG. 17C). Next, this assay's performance was evaluated with the same 17 SARS-CoV-2 RNA samples used in FIG. 17A and the results from the visual-based detection assay were compared to the results in FIG. 17A. There was 100% concordance between the two assays, indicating that the developed mCas13 visual detection assay is reliable (FIG. 17D). Finally, to clinically validate and further test the reliability of the visual mCas13 detection assay, the assay's performance was tested with an additional 24 qPCR-confirmed positive clinical samples of total RNA extracted from patient swabs, along with no-template control reactions. The visual-readout mCas13-based detection of SARS-CoV-2 from these 24 samples showed 100% concordance with RT-qPCR results. A clear fluorescence signal relative to the negative controls was detected in each positive sample (FIGS. 17E-17F). Altogether, this system's effectiveness was validated in 41 clinical samples, facilitating viral detection within 1 hour with a simple visual readout.

Discussion

To control the pandemic of COVID-19, rapid, accurate, reliable, and portable diagnostics for SARS-CoV-2 are needed [66]. However, increasing sensitivity and specificity has remained a common challenge for recently developed diagnostics Amplification can increase the sensitivity of assays and isothermal amplification techniques like RT-LAMP and RT-RPA are good alternatives for RT-PCR. Both are highly sensitive, provide rapid and more accessible platforms for viral nucleic acid detection, and are suitable for POC uses [111-113]. Despite many advantages of isothermal amplification in relation to other traditional amplification methods, their application is limited by the high rate of non-specific amplification and cross-contamination [114, 115]. Therefore, isothermal amplification methods are coupled with CRISPR systems to enhance specificity and LoD [81, 86, 116, 117].

Here, a previously unreported/uncharacterized Cas13 variant was identified and characterized and an RT-LAMP coupled with mCas13 assay for SARS-CoV-2 detection was developed. Sequence similarity searching was used to identify the mCas13 variant. The corresponding mCas13 sequence was synthesized and subcloned into a protein expression vector in order to purify the protein and characterize its cleavage activity. The crRNA architecture was identified and synthesized for in vitro expression, and the crRNA and mCas13 were used to form an RNP complex for cleavage activity assays. multiple crRNAs targeting different regions in SARS-CoV-2 were designed. One crRNA showed robust collateral activity against poly(U) reporters. Different regions of the crRNA exhibited different tolerances for mismatches, which can be advantageous to designing crRNAs useful for detecting variants that harbor different SNPs.

Because mCas13 targets RNA for cleavage and degradation, the LAMP primers were modified to add the T7 polymerase promoter sequence to permit the T7 RNA polymerase to generate transcripts for mCas13. This modified RT-LAMP enabled the coupling with mCas13 for virus detection, obviating the need for extra steps. This modification in the RT-LAMP primers did not interfere with the RT-LAMP reaction and resulted in robust amplification of the target sequences. The RT-LAMP coupled with mCas13 exhibited high specificity since sequences of other viruses, including SARS-Co-V1, MERS, TMV, and TuMV, did not trigger the collateral reaction; however, the SARS-CoV2 sequence triggered a strong collateral cleavage reaction and fluorescence.

The usefulness of this modality (RT-LAMP coupled with mCas13) for SARS-CoV-2 detection was tested and validated in 41 clinical samples. The data showed that mCas13 successfully detected SARS-CoV-2 in clinical samples, exhibiting strong concordance with RT-qPCR. A simple LED-based visualizer was employed for straightforward and inexpensive detection of fluorescence in test results. With the affordable and portable P51 LED-based visualizer, a positive signal is observable as a bright green light. The simple fluorescence visualizer showed consistent detection results from fluorescence readers, verifying the effectiveness of the method. It is worth noting that several crRNAs were screened to identify functional ones that activated mCas13, thereby limiting the off-target activities of the mCas13 enzyme. Other CRISPR systems with robust activities can degrade the target and compromise the detection sensitivity. Because this mCas13 system is not overly robust, it may be quite useful for sensitive and specific detection. These features, however, could be quite beneficial in building a highly specific and powerful modality of virus and nucleic acid detection. Briefly, this mCas13-based detection platform enables rapid, accurate, simple, cost-effective, and efficient detection of SARS-CoV-2, and shows potential for POC applications.

Due to its miniature size, the mCas13 variant can be used for a variety of in vivo RNA manipulations, including RNA knockdown, editing, splicing regulation, RNA imaging and localization. The recently identified compact Cas13, Cas13e.1, shows efficacy against SAR-CoV-2 and influenza A virus, indicating that Cas13 can be useful as an antiviral therapeutic [101, 107]. These studies demonstrate that bacterial defense systems have untapped potential for diverse synthetic biology applications, diagnostics, and therapeutics as antiviral agents.

In summary, the data demonstrate successful identification and characterization of the catalytic activities of a previously unknown/uncharacterized miniature variant of Cas13, and harnessing of its collateral catalytic activities to develop a system for SARS-CoV-2 detection. The modality coupling RT-LAMP and mCas13 demonstrates key features, including simplicity, specificity, sensitivity, and portability. The readout signal was measured using a low-cost P51 device. The P51 device can be paired with a cell phone camera that processes and shares data, facilitating the integration of this modality to large-scale testing. This work illustrates the usefulness of mCas13 systems for diagnostics as well as other potential in vivo RNA manipulations for diverse applications.

### REFERENCES

1. Zhou, P., et al., A pneumonia outbreak associated with a new coronavirus of probable bat origin. Nature, 2020. 579(7798): p. 270-273.
2. Zhu, N., et al., A Novel Coronavirus from Patients with Pneumonia in China, 2019. N Engl J Med, 2020. 382(8): p. 727-733.
3. Huang, C., et al., Clinical features of patients infected with 2019 novel coronavirus in Wuhan, China. Lancet, 2020. 395(10223): p. 497-506.
4. Cui, J., F. Li, and Z. L. Shi, Origin and evolution of pathogenic coronaviruses. Nat Rev Microbiol, 2019. 17(3): p. 181-192.
5. Cotten, M., et al., Full-genome deep sequencing and phylogenetic analysis of novel human betacoronavirus. Emerg Infect Dis, 2013. 19(5): p. 736-42B.
6. Chan, J. F., et al., Genomic characterization of the 2019 novel human-pathogenic coronavirus isolated from a patient with atypical pneumonia after visiting Wuhan. Emerg Microbes Infect, 2020. 9(1): p. 221-236.
7. Wu, F., et al., A new coronavirus associated with human respiratory disease in China. Nature, 2020. 579(7798): p. 265-269.
8. Lu, R., et al., Genomic characterisation and epidemiology of 2019 novel coronavirus: implications for virus origins and receptor binding. Lancet, 2020. 395(10224): p. 565-574.
9. Kim, D., et al., The Architecture of SARS-CoV-2 Transcriptome. Cell, 2020.
10. Li, Q., et al., Early Transmission Dynamics in Wuhan, China, of Novel Coronavirus-Infected Pneumonia. N Engl J Med, 2020. 382(13): p. 1199-1207.
11. Han, Y. and H. Yang, The transmission and diagnosis of 2019 novel coronavirus infection disease (COVID-19): A Chinese perspective. J Med Virol, 2020.
12. Jones, G., et al., The French human Salmonella surveillance system: evaluation of timeliness of laboratory reporting and factors associated with delays, 2007 to 2011. Euro Surveill, 2014. 19(1).
13. Isere, E. E., A. A. Fatiregun, and I. O. Ajayi, An overview of disease surveillance and notification system in Nigeria and the roles of clinicians in disease outbreak prevention and control. Niger Med J, 2015. 56(3): p. 161-8.
14. Niemz, A., T. M. Ferguson, and D.S. Boyle, Point-of-care nucleic acid testing for infectious diseases. Trends Biotechnol, 2011. 29(5): p. 240-50.
15. Dohla, M., et al., Rapid point-of-care testing for SARS-CoV-2 in a community screening setting shows low sensitivity. Public Health, 2020. 182: p. 170-172.
16. Corman, V. M., et al., Detection of 2019 novel coronavirus (2019—nCoV) by real-time RT-PCR. Euro Surveill, 2020. 25(3).
17. Morshed, M. G., et al., Molecular methods used in clinical laboratory: prospects and pitfalls. FEMS Immunol Med Microbiol, 2007. 49(2): p. 184-91.
18. Nguyen, T., D. Duong Bang, and A. Wolff, 2019 Novel Coronavirus Disease (COVID-19): Paving the Road for Rapid Detection and Point-of-Care Diagnostics. Micromachines (Basel), 2020. 11(3).
19. Mabey, D., et al., Diagnostics for the developing world. Nat Rev Microbiol, 2004. 2(3): p. 231-40.
20. Mori, Y. and T. Notomi, Loop-mediated isothermal amplification (LAMP): a rapid, accurate, and cost-effective diagnostic method for infectious diseases. J Infect Chemother, 2009. 15(2): p. 62-9.
21. Notomi, T., et al., Loop-mediated isothermal amplification of DNA. Nucleic Acids Res, 2000. 28(12): p. E63.
22. Chou, P. H., et al., Real-time target-specific detection of loop-mediated isothermal amplification for white spot syndrome virus using fluorescence energy transfer-based probes. J Virol Methods, 2011. 173(1): p. 67-74.
23. Nagai, K., et al., Diagnostic test accuracy of loop-mediated isothermal amplification assay for Mycobacterium tuberculosis: systematic review and meta-analysis. Sci Rep, 2016. 6: p. 39090.
24. Barrangou, R. and L. A. Marraffini, CRISPR-Cas systems: Prokaryotes upgrade to adaptive immunity. Mol Cell, 2014. 54(2): p. 234-44.
25. Sorek, R., C. M. Lawrence, and B. Wiedenheft, CRISPR-mediated adaptive immune systems in bacteria and archaea. Annu Rev Biochem, 2013. 82: p. 237-66.
26. Mahas, A., R. Aman, and M. Mahfouz, CRISPR-Cas13d mediates robust RNA virus interference in plants. Genome Biol, 2019. 20(1): p. 263.
27. Doudna, J.A. and E. Charpentier, Genome editing. The new frontier of genome engineering with CRISPR-Cas9. Science, 2014. 346(6213): p. 1258096.
28. Mahas, A., C. Neal Stewart, Jr., and M. M. Mahfouz, Harnessing CRISPR/Cas systems for programmable transcriptional and post-transcriptional regulation. Biotechnol Adv, 2017.
29. Abudayyeh, O. O., et al., C2c2 is a single-component programmable RNA-guided RNA-targeting CRISPR effector. Science, 2016. 353(6299): p. aaf5573.
30. Chen, J. S., et al., CRISPR-Cas12a target binding unleashes indiscriminate single-stranded DNase activity. Science, 2018. 360(6387): p. 436-439.
31. Harrington, L. B., et al., Programmed DNA destruction by miniature CRISPR-Cas14 enzymes. Science, 2018. 362(6416): p. 839-842.
32. Aman, R., A. Mahas, and M. Mahfouz, Nucleic Acid Detection Using CRISPR/Cas Biosensing Technologies. ACS Synth Biol, 2020.
33. Li, Y., et al., CRISPR/Cas Systems towards Next-Generation Biosensing. Trends Biotechnol, 2019. 37(7): p. 730-743.
34. Kellner, M. J., et al., SHERLOCK: nucleic acid detection with CRISPR nucleases. Nat Protoc, 2019. 14(10): p. 2986-3012.
35. Milligan, J. N., et al., Evolution of a Thermophilic Strand-Displacing Polymerase Using High-Temperature Isothermal Compartmentalized Self-Replication. Biochemistry, 2018. 57(31): p. 4607-4619.
36. Ellefson, J. W., et al., Synthetic evolutionary origin of a proofreading reverse transcriptase. Science, 2016. 352(6293): p. 1590-3.
37. Zhang, Y., et al., Rapid Molecular Detection of SARS-CoV-2 (COVID-19) Virus RNA Using Colorimetric LAMP. medRxiv, 2020.
38. Teng, F., et al., Repurposing CRISPR-Cas12b for mammalian genome engineering. Cell Discov, 2018. 4: p. 63.
39. Shmakov, S., et al., Discovery and Functional Characterization of Diverse Class 2 CRISPR-Cas Systems. Mol Cell, 2015. 60(3): p. 385-97.
40. Bhadra, S., et al., Cellular reagents for diagnostics and synthetic biology. PLoS One, 2018. 13(8): p. e0201681.
41. Wyllie, A. L., et al., Saliva is more sensitive for SARS-CoV-2 detection in COVID-19 patients than nasopharyngeal swabs. medRxiv, 2020.
42. Ma, Y., et al., Enhancement of Polymerase Activity of the Large Fragment in DNA Polymerase I from Geobacillus stearothermophilus by Site-Directed Mutagenesis at the Active Site. Biomed Res Int, 2016. 2016: p. 2906484.
43. Bhadra, S., A. C. Maranhao, and A. D. Ellington, A one-enzyme RT-qPCR assay for SARS-CoV-2, and procedures for reagent production. bioRxiv, 2020.
44. Wang, Y., et al., A One-Pot Toolbox Based on Cas12a/crRNA Enables Rapid Foodborne Pathogen Detection at Attomolar Level. ACS Sens, 2020.
45. Wang, B., et al., Cas12aVDet: A CRISPR/Cas12a-Based Platform for Rapid and Visual Nucleic Acid Detection. Analytical Chemistry, 2019. 91(19): p. 12156-12161. 46. Cui, J., F. Li, and Z. L. Shi, Origin and evolution of pathogenic coronaviruses. Nat Rev Microbiol, 2019. 17(3): p. 181-192.
47. Kilic, T., R. Weissleder, and H. Lee, Molecular and Immunological Diagnostic Tests of COVID-19: Current Status and Challenges. iScience, 2020. 23(8): p. 101406.
48. Mabey, D., et al., Diagnostics for the developing world. Nat Rev Microbiol, 2004. 2(3): p. 231-40.
49. Broughton, J. P., et al., CRISPR-Cas12-based detection of SARS-CoV-2. Nat Biotechnol, 2020. 38(7): p. 870-874.
50. Kellner, M. J., et al., SHERLOCK: nucleic acid detection with CRISPR nucleases. Nature protocols, 2019. 14(10): p. 2986-3012.
51. Gootenberg, J. S., et al., Multiplexed and portable nucleic acid detection platform with Cas13, Cas12a, and Csm6. Science, 2018. 360(6387): p. 439-444.
52. Gootenberg, J. S., et al., Nucleic acid detection with CRISPR-Cas13a/C2c2. Science, 2017.
53. Abudayyeh, O. O., et al., C2c2 is a single-component programmable RNA-guided RNA-targeting CRISPR effector. Science, 2016. 353(6299): p. aaf5573.
54. East-Seletsky, A., et al., Two distinct RNase activities of CRISPR-C2c2 enable guide-RNA processing and RNA detection. Nature, 2016. 538(7624): p. 270-273.
55. East-Seletsky, A., et al., RNA Targeting by Functionally Orthogonal Type VI-A CRISPR-Cas Enzymes. Mol Cell, 2017. 66(3): p. 373-383 e3.
56. Thompson, D. and Y. Lei, Mini review: Recent progress in RT-LAMP enabled COVID-19 detection. Sensors and Actuators Reports, 2020. 2(1): p. 100017.
57. Joung, J., et al., Detection of SARS-CoV-2 with SHERLOCK One-Pot Testing. N Engl J Med, 2020. 383(15): p. 1492-1494.
58. Arizti-Sanz, J., et al., Streamlined inactivation, amplification, and Cas13-based detection of SARS-CoV-2. Nat Commun, 2020. 11(1): p. 5921.
59. Ali, Z., et al., iSCAN: An RT-LAMP-coupled CRISPR-Cas12 module for rapid, sensitive detection of SARS-CoV-2. Virus Res, 2020. 288: p. 198129.
60. Sun, Y., et al., One-tube SARS-CoV-2 detection platform based on RT-RPA and CRISPR/Cas12a. J Transl Med, 2021. 19(1): p. 74.
61. Joung, J., et al., Detection of SARS-CoV-2 with SHERLOCK One-Pot Testing. New England Journal of Medicine, 2020. 383(15): p. 1492-1494.
62. Fozouni, P., et al., Amplification-free detection of SARS-CoV-2 with CRISPR-Cas13a and mobile phone microscopy. Cell, 2021. 184(2): p. 323-333 e9.
63. Liu, T. Y., et at, Accelerated RNA detection using tandem CRISPR nucleases. 2021.
64. Lopato, S., et al., atSRp30, one of two SF2/ASF-like proteins from Arabidopsis thaliana, regulates splicing of specific plant genes. Genes Dev, 1999 Regarding the significance of AS in plant development. 13(8): p. 987-1001.
65. Kellner, M. J., et al., SHERLOCK: nucleic acid detection with CRISPR nucleases. Nat Protoc, 2019. 14(10): p. 2986-3012.
66. Kelly-Cirino, C. D., et al., Importance of diagnostics in epidemic and pandemic preparedness. BMJ Glob Health, 2019. 4(Suppl 2): p. e001179.
67. McDermott, J. H., et al., The rise of point-of-care genetics: how the SARS-CoV-2 pandemic will accelerate adoption of genetic testing in the acute setting. Eur J Hum Genet, 2021.
68. Esbin, M. N., et al., Overcoming the bottleneck to widespread testing: a rapid review of nucleic acid testing approaches for COVID-19 detection. RNA, 2020. 26(7): p. 771-783.
69. Niemz, A., T. M. Ferguson, and D.S. Boyle, Point-of-care nucleic acid testing for infectious diseases. Trends Biotechnol, 2011. 29(5): p. 240-50.
70. Corman, V. M., et al., Detection of 2019 novel coronavirus (2019-nCoV) by real-time RT-PCR. Euro Surveill, 2020. 25(3).
71. Morshed, M. G., et al., Molecular methods used in clinical laboratory: prospects and pitfalls. FEMS Immunol Med Microbiol, 2007. 49(2): p. 184-91.
72. Vogels, C. B. F., et al., Analytical sensitivity and efficiency comparisons of SARS-CoV-2 RT-qPCR primer-probe sets. Nat Microbiol, 2020. 5(10): p. 1299-1305.
73. Larremore, D. B., et al., Test sensitivity is secondary to frequency and turnaround time for COVID-19 surveillance. medRxiv, 2020.
74. Piepenburg, O., et al., DNA detection using recombination proteins. PLoS Biol, 2006. 4(7): p. e204.
75. Notomi, T., et al., Loop-mediated isothermal amplification of DNA. Nucleic Acids Res, 2000. 28(12): p. E63.
76. Obande, G. A. and K. K. Banga Singh, Current and Future Perspectives on Isothermal Nucleic Acid Amplification Technologies for Diagnosing Infections. Infect Drug Resist, 2020. 13: p. 455-483.
77. Ning, B., et al., A smartphone-read ultrasensitive and quantitative saliva test for COVID-19. Sci Adv, 2021. 7(2).
78. Patchsung, M., et al., Clinical validation of a Cas13-based assay for the detection of SARS-CoV-2 RNA. Nat Biomed Eng, 2020. 4(12): p. 1140-1149.
79. Arizti-Sanz, J., et al., Streamlined inactivation, amplification, and Cas13-based detection of SARS-CoV-2. Nat Commun, 2020. 11(1): p. 5921.
80. Ali, Z., et al., iSCAN: An RT-LAMP-coupled CRISPR-Cas12 module for rapid, sensitive detection of SARS-CoV-2. Virus Res, 2020. 288: p. 198129.
81. Joung, J., et al., Detection of SARS-CoV-2 with SHERLOCK One-Pot Testing. N Engl J Med, 2020. 383(15): p. 1492-1494.
82. Broughton, J. P., et al., CRISPR-Cas12-based detection of SARS-CoV-2. Nat Biotechnol, 2020. 38(7): p. 870-874.
83. Marsic, T., et al., Vigilant: An Engineered VirD2-Cas9 Complex for Lateral Flow Assay-Based Detection of SARS-CoV2. Nano Lett, 2021.
84. Aman, R., A. Mahas, and M. Mahfouz, Nucleic Acid Detection Using CRISPR/Cas Biosensing Technologies. ACS Synth Biol, 2020.
85. Li, Y., et al., CRISPR/Cas Systems towards Next-Generation Biosensing. Trends Biotechnol, 2019. 37(7): p. 730-743.
86. Gootenberg, J. S., et al., Nucleic acid detection with CRISPR-Cas13a/C2c2. Science, 2017.
87. Kellner, M. J., et al., SHERLOCK: nucleic acid detection with CRISPR nucleases. Nat Protoc, 2019. 14(10): p. 2986-3012.
88. Gootenberg, J. S., et al., Multiplexed and portable nucleic acid detection platform with Cas13, Cas12a, and Csm6. Science, 2018. 360(6387): p. 439-444.
89. Myhrvold, C., et al., Field-deployable viral diagnostics using CRISPR-Cas13. Science, 2018. 360(6387): p. 444-448.
90. Mabey, D., et al., Diagnostics for the developing world. Nat Rev Microbiol, 2004. 2(3): p. 231-40.
91. Liu, T. Y., et at, Accelerated RNA detection using tandem CRISPR nucleases. medRxiv, 2021.
92. Fozouni, P., et al., Amplification-free detection of SARS-CoV-2 with CRISPR-Cas13a and mobile phone microscopy. Cell, 2021. 184(2): p. 323-333 e9.
93. Burstein, D., et al., New CRISPR-Cas systems from uncultivated microbes. Nature, 2017. 542(7640): p. 237-241.
94. Al-Shayeb, B., et al., Clades of huge phages from across Earth's ecosystems. Nature, 2020. 578(7795): p. 425-431.
95. Smargon, A. A., et al., Cas13b Is a Type VI-B CRISPR-Associated RNA-Guided RNase Differentially Regulated by Accessory Proteins Csx27 and Csx28. Mol Cell, 2017. 65(4): p. 618-630 e7.
96. Yan, W. X., et al., Cas13d Is a Compact RNA-Targeting Type VI CRISPR Effector Positively Modulated by a WYL-Domain-Containing Accessory Protein. Mol Cell, 2018. 70(2): p. 327-339 e5.
97. Shmakov, S., et al., Discovery and Functional Characterization of Diverse Class 2 CRISPR-Cas Systems. Mol Cell, 2015. 60(3): p. 385-97.
98. Konermann, S., et al., Transcriptome Engineering with RNA-Targeting Type VI-D CRISPR Effectors. Cell, 2018. 173(3): p. 665-676 e14.
99. Abudayyeh, O. O., et al., RNA targeting with CRISPR-Cas13. Nature, 2017. 550(7675): p. 280-284.
100. Cox, D. B. T., et al., RNA editing with CRISPR-Cas13. Science, 2017.
101. Abbott, T. R., et al., Development of CRISPR as an Antiviral Strategy to Combat SARS-CoV-2 and Influenza. Cell, 2020. 181(4): p. 865-876 e12.
102. Mahas, A., R. Aman, and M. Mahfouz, CRISPR-Cas13d mediates robust RNA virus interference in plants. Genome Biol, 2019. 20(1): p. 263.
103. Aman, R., et al., RNA virus interference via CRISPR/Cas13a system in plants. Genome Biol, 2018. 19(1): p. 1.
104. Ackerman, C. M., et al., Massively multiplexed nucleic acid detection with Cas13. Nature, 2020. 582(7811): p. 277-282.
105. Freije, C. A., et al., Programmable Inhibition and Detection of RNA Viruses Using Cas13. Mol Cell, 2019. 76(5): p. 826-837 e11.
106. Brogan, D. J., et al., A Sensitive, Rapid, and Portable CasRx-based Diagnostic Assay for SARS-CoV-2. medRxiv, 2020.
107. Xu, C., et al., Novel miniature CRISPR-Cas13 systems from uncultivated microbes effective in degrading SARS-CoV-2 sequences and influenza viruses. Research Square, 2021. DOI: 10.21203/rs.3.rs-30924/v1. https://www.researchsquare.com/article/rs-30924/v1.
108. Abudayyeh, O. O., et al., C2c2 is a single-component programmable RNA-guided RNA-targeting CRISPR effector. Science, 2016. 353(6299): p. aaf5573.
109. East-Seletsky, A., et al., Two distinct RNase activities of CRISPR-C2c2 enable guide-RNA processing and RNA detection. Nature, 2016. 538(7624): p. 270-273.
110. Thompson, D. and Y. Lei, Mini review: Recent progress in RT-LAMP enabled COVID-19 detection. Sensors and Actuators Reports, 2020. 2(1): p. 100017.
111. Dao Thi, V. L., et al., A colorimetric RT-LAMP assay and LAMP-sequencing for detecting SARS-CoV-2 RNA in clinical samples. Sci Transl Med, 2020. 12(556).
112. Ganguli, A., et at, Rapid Isothermal Amplification and Portable Detection System for SARS-CoV-2. bioRxiv, 2020.
113. Qian, J., et al., An enhanced isothermal amplification assay for viral detection. bioRxiv, 2020.
114. Nagai, K., et al., Diagnostic test accuracy of loop-mediated isothermal amplification assay for Mycobacterium tuberculosis: systematic review and meta-analysis. Sci Rep, 2016. 6: p. 39090.
115. Chou, P. H., et al., Real-time target-specific detection of loop-mediated isothermal amplification for white spot syndrome virus using fluorescence energy transfer-based probes. J Virol Methods, 2011. 173(1): p. 67-74.
116. Aman, R., et al., Efficient, Rapid, and Sensitive Detection of Plant RNA Viruses With One-Pot RT-RPA-CRISPR/Cas12a Assay. Front Microbiol, 2020. 11: p. 610872.
117. Mahas, A., et al., LAMP-Coupled CRISPR-Cas12a Module for Rapid and Sensitive Detection of Plant DNA Viruses. Viruses, 2021. 13(3).
118. Larkin, M. A., et al., Clustal W and Clustal X version 2.0. Bioinformatics, 2007. 23(21): p. 2947-8.
119. Gouet, P., et al., ESPript: analysis of multiple sequence alignments in PostScript. Bioinformatics, 1999. 15(4): p. 305-8.
120. Couvin, D., et al., CRISPRCasFinder, an update of CRISRFinder, includes a portable version, enhanced performance and integrates search for Cas proteins. Nucleic Acids Res, 2018. 46(W1): p. W246-W251.
121. Biswas, A., et al., CRISPRDetect: A flexible algorithm to define CRISPR arrays. BMC Genomics, 2016. 17: p. 356.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Although the description of materials, compositions, components, steps, techniques, etc. may include numerous options and alternatives, this should not be construed as, and is not an admission that, such options and alternatives are equivalent to each other or, in particular, are obvious alternatives.

Every composition disclosed herein is intended to be and should be considered to be specifically disclosed herein. Further, every subgroup that can be identified within this disclosure is intended to be and should be considered to be specifically disclosed herein. As a result, it is specifically contemplated that any composition, or subgroup of compositions can be either specifically included for or excluded from use or included in or excluded from a list of compositions.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of detecting the presence of SARS-CoV-2 nucleic acid in a sample, comprising:
(a) contacting the sample in an RT-LAMP reaction with a set of reverse transcription-loop-mediated isothermal amplification (RT-LAMP) primers specific for the N or E gene of SARS-CoV-2 under conditions sufficient for amplification of the N or E gene of SARS-CoV-2 to generate an amplification product,
(b) contacting the product from step (a) with (i) a composition comprising a ribonucleoprotein (RNP) comprising a DNA editing enzyme with double stranded DNA cleavage activity and single stranded DNA cleavage activity and a crRNA complementary to the amplification product from step (a) and (ii) an activatable single stranded DNA (ssDNA) oligonucleotide comprising a reporter moiety,
(c) detecting cleavage of the ssDNA oligonucleotide by the DNA editing enzyme, wherein the cleavage is dependent on or subsequent to binding of the RNP to the RT-LAMP amplification product, thereby detecting the presence of the SARS-CoV-2 nucleic acid.

2. The method of claim 1, wherein the DNA editing enzyme is a type V CRISPR/Cas effector protein.

3. The method of claim 1 or 2, wherein each primer in the set of primers comprise a nucleic acid sequence selected from the group consisting of SEQ ID NOS: 11-28.

4. The method of any one of claims 1-3, wherein each primer in the set of primers comprise a nucleic acid sequence having at least 90% sequence identity to any one of SEQ ID NOs: 11-28.

5. The method of claim 3 or 4, wherein each primer in the set of primers comprise a nucleic acid sequence selected from the group consisting of SEQ ID Nos: 17-22.

6. The method of any one of claims 1-5, wherein the DNA editing enzyme is a Cas12 effector protein.

7. The method of claim 6, wherein the DNA editing enzyme is selected from a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide.

8. The method of any one of claims 1-7, wherein the sample is selected from the group consisting of mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood,
optionally wherein the method further comprises extracting nucleic acids from the sample prior to step (a) and contacting the nucleic acids with the RT-LAMP primers in step (a).

9. The method of claim 8, wherein the sample is obtained from a nasopharyngeal swab, a nasopharyngeal aspirate, sputa/deep throat saliva, or a throat swab.

10. The method of any one of claims 1-9, wherein the RT-LAMP reaction conditions comprise a reaction temperature of about 60° C. and primer concentrations: about 2 μM F3 (SEQ ID NO: 11, 17 or 23), about 2 μM B3 (SEQ ID NO: 12, 18 or 24), about 16 μM FIP (SEQ ID NO: 13, 19 or 25), about 16 μM BIP (SEQ ID NO: 14, 20 or 26), about 8 μM LF (SEQ ID NO. 15, 21 or 27), and about 8 μM LB (SEQ ID NO: 16, 22 or 28).

11. The method of claim 10, wherein the RT-LAMP reaction comprises a reverse transcriptase and a DNA polymerase and wherein the RT-LAMP reaction proceeds under conditions effective to amplify the N or E genes of Sars-CoV-2, resulting in an amplification product.

12. The method of any one of claims 1-11, wherein the DNA editing enzyme comprises Cas12a, preferably LbCas12a.

13. The method of any one of claims 1-11, wherein the DNA editing enzyme comprises Cas12b, preferably AacCas12b or AapCas12b.

14. The method of any one of claims 1-13, wherein the RNP is formed by contacting the DNA editing enzyme with the crRNA complementary to the RT-LAMP amplification product and wherein the method comprises contacting the amplification product with the RNP in the presence of the activatable single stranded DNA oligonucleotide.

15. The method of any one of claims 1-14, wherein the activatable single stranded DNA oligonucleotide comprises a fluorophore-quencher pair.

16. The method of any one of claims 1-15, wherein cleavage of the ssDNA oligonucleotide is detected by a signal from the reporter moiety, preferably the fluorophore, thereby indicating the presence of SARS-CoV-2 in the sample.

17. The method of claim 16, wherein the signal is fluorescence, wherein the fluorescence is emitted upon cleavage of the ssDNA oligonucleotide.

18. The method of any one of claims 1-14, wherein the reporter moiety comprises FAM and/or biotin moieties, wherein cleavage of the ssDNA oligonucleotide is detected by a lateral flow assay.

19. The method of any one of claims 1-18, wherein steps (a), (b), and (c) are performed in the same container.

20. A method of detecting the presence of SARS-CoV-2 nucleic acid in a sample, comprising:
(a) contacting the sample in an RT-LAMP reaction with a set of reverse transcription-loop-mediated isothermal amplification (RT-LAMP) primers specific for the N or E gene of SARS-CoV-2 under conditions sufficient for amplification of the N or E gene of SARS-CoV-2 to generate an amplification product;
(b) transcribing the amplification product to generate an RNA transcript;
(c) contacting the RNA transcript with
(i) a composition comprising a Cas13-based RNP complex comprising a Cas13 enzyme and a crRNA complementary to the RNA transcript; and
(ii) an activatable single stranded RNA (ssRNA) oligonucleotide; and
(d) detecting cleavage of the ssRNA oligonucleotide by the Cas13 enzyme, thereby detecting the presence of the SARS-CoV-2 nucleic acid.

21. The method of claim 20, wherein the ssRNA oligonucleotide is cleaved subsequent to binding of the Cas13-based RNP complex to the RNA transcript.

22. The method of claim 20 or 21, wherein the ssRNA oligonucleotide comprises a mixed nucleotide sequence or a homopolymeric sequence, preferably poly(U).

23. The method of any one of claims 20-22, wherein the crRNA comprises a spacer of about 20-30 nucleotides, preferably 24-28 nucleotides.

24. The method of any one of claims 20-23, wherein the crRNA is encoded by the nucleic acid sequence of SEQ ID NO:61 or 75.

25. The method of any one of claims 20-24, wherein each primer in the set of primers comprise a nucleic acid sequence selected from the group consisting of SEQ ID NOs: 29-44.

26. The method of any one of claims 20-25, wherein the primers comprise the nucleic acid sequences of SEQ ID NOs: 43 and 44.

27. The method of any one of claims 20-26, wherein the ssRNA oligonucleotide comprises a fluorophore-quencher pair, wherein fluorescence is emitted upon cleavage of the ssRNA oligonucleotide, thereby indicating the presence of the SARS-CoV-2 nucleic acid in the sample.

28. The method of claim 27, wherein the fluorophore comprises hexachloro-fluorescein (HEX), wherein the fluorescence is detected by a portable or hand-held fluorescence visualizer.

29. The method of any one of claims 20-28, wherein the amplification product is transcribed in vitro by a T7 RNA polymerase.

30. The method of any one of claims 20-29, wherein the Cas13 enzyme is a Cas13a protein derived from thermophilic bacteria, preferably from Thermoclostridium caenicola (TccCas13a) or Herbinix hemicellulosilytica (HheCas13a).

31. The method of any one of claims 20-30, wherein the Cas13 enzyme comprises the amino acid sequence of SEQ ID NO:67 or SEQ ID NO:95, or a sequence having at least 70% sequence identity to SEQ ID NO:67 or SEQ ID NO:95.

32. The method of claim 30 or 31, wherein the Cas13 enzyme exhibits ssRNA cleavage activity at a temperature of about 37-70° C., preferably about 47-60° C., more preferably about 60° C.

33. The method of any one of claims 30-32, wherein steps (a)-(c) are performed in one container, preferably at a temperature of about 56° C.

34. The method of claim 33, wherein
(i) the Cas13-based RNP complex is present in a concentration of about 12-100 nM;
(ii) the RNA polymerase is present in a concentration of about 2-4 U/ul;
(iii) the DNA polymerase used in the RT-LAMP reaction is present in a concentration of about 0.1-0.8 U/ul;
(iv) MgSO₄ is present in a concentration of about 8 mM; or a combination thereof.

35. The method of any one of claims 20-29, wherein the Cas13 enzyme is derived from a Proteobacteria bacterium.

36. The method of any one of claim 20-29 or 35, wherein the Cas13 enzyme comprises the amino acid sequence of SEQ ID NO:68 or a sequence having at least 70% sequence identity to SEQ ID NO:68.

37. The method of claim 35 or 36, wherein the Cas13 enzyme exhibits ssRNA cleavage activity at a temperature of about 37° C.-42° C., preferably about 37° C.

38. The method of any one of claims 35-37, wherein the Cas13-based RNP complex is present in a concentration of about 400-500 nM, preferably about 500 nM.

39. The method of any one of claims 35-38, wherein step (a) is performed in a first container, and steps (b)-(c) are performed in a second container.

40. The method of claim 39, wherein step (a) is performed at a temperature of about 62° C., and steps (b)-(c) are performed at a temperature of about 37° C.

41. The method of any one of claims 20-40, wherein the sample comprises nucleic acid derived or isolated from mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood.

42. The method of any one of claims 20-41, wherein the method is performed in about 60-120 minutes, preferably about 60-80 minutes, more preferably about 60 minutes.

43. The method of any one of claims 1-42, wherein the method has a limit of detection of about 2-20 copies/μL of the SARS-CoV-2 nucleic acid, preferably about 4-8 copies/μL or 5-10 copies/μL.

44. A method of detecting the presence of a target nucleic acid in a sample, comprising:
(a) contacting the sample in an RT-LAMP reaction with a set of primers specific to the target nucleic acid under conditions sufficient for amplification of the target nucleic acid to generate an amplification product;
(b) transcribing the amplification product to generate an RNA transcript;
(c) contacting the RNA transcript with
(i) a RNP complex comprising a Cas13 enzyme comprising the sequence of any one of SEQ ID NO:67, 68, or 95 or a sequence having at least 70% sequence identity to any one of SEQ ID NO:67, 68, or 95, and a crRNA complementary to the RNA transcript; and
(ii) an activatable single stranded RNA (ssRNA) oligonucleotide; and
(d) detecting cleavage of the ssRNA oligonucleotide by the Cas13 enzyme, thereby detecting the presence of the target nucleic acid.

45. The method of claim 44, wherein the target nucleic acid is associated with a disease, optionally wherein the disease is selected from an infection, an organ disease, a blood disease, an immune system disease, a cancer, a brain and nervous system disease, an endocrine disease, a pregnancy or childbirth-related disease, an inherited disease, or an environmentally-acquired disease.

46. The method of claim 44 or 45, wherein the target nucleic acid is associated with a fungal infection, a bacterial infection, a parasitic infection, a viral infection, or combination thereof.

47. The method of any one of claims 44-46, wherein the target nucleic acid is associated with a pathogen.
